(19) United States
(12) Patent Application Publication
(10) Pub. No.: US 2014/0073589 A1
(43) Pub. Date: Mar. 13, 2014

(54) Amphetamine Prodrugs

(71) Applicant: Shire AG, Eysins (CH)

(72) Inventors: Rhys WHOMSLEY, Alton (GB); Christine Elizabeth Allan, Eastleigh (GB); Timothy Jon Luker, Nottingham (GB)

(21) Appl. No.: 13/928,049

(22) Filed: Jun. 26, 2013

Related U.S. Application Data
(60) Provisional application No. 61/665,093, filed on Jun. 27, 2012.
(60) Provisional application No. 61/788,314, filed on Mar. 15, 2013.

(51) Int. Cl. A61K 47/48

(52) U.S. Cl. CPC A61K 47/48092
USPC 514/25; 536/53; 536/17.2; 514/23

(57) ABSTRACT

The present invention relates to amphetamine prodrugs which provide colonic release of amphetamine.

This application claims the benefit of U.S. Provisional Application Nos. 61/665,093, filed Jun. 27, 2012, and 61/788,314, filed Mar. 15, 2013, both of which are hereby incorporated by reference.

### FIELD OF THE INVENTION

The present invention relates to amphetamine prodrugs which provide colonic release of amphetamine.

### BACKGROUND OF THE INVENTION

Stimulants, such as amphetamines, enhance the activity of the sympathetic nervous system and/or central nervous system (CNS) and are prescribed for the treatment of a variety of disorders, such as attention deficit hyperactivity disorder (ADHD).

The potential for abuse of amphetamines is a major drawback to their use. Amphetamine can be abused orally or the tablets can be crushed and snorted or dissolved in water and injected. The high abuse potential has earned it Schedule II status according to the Controlled Substances Act (CSA). Schedule II classification is reserved for those drugs that have accepted medical use but have the highest potential for abuse. The abuse potential of amphetamine has been known for many years and the U.S. FDA requires a black box warning advising that amphetamines have a high potential for abuse, prolonged administration may lead to dependence, and misuse of amphetamines may cause sudden death and serious cardiovascular adverse reactions.

Rendering amphetamines resistant to abuse, particularly by parenteral routes such as snorting or injecting, would provide considerable value to this otherwise effective and beneficial medication.

Dosing of amphetamine products in the morning is often inconvenient, and is not always easily accommodated in certain situations, for example, children who are rushing off to school. This may lead to poor patient compliance. Furthermore, existing amphetamine products may not provide sufficiently early onset of action for some patients. Therefore, there exists a need for forms of amphetamine which will provide an earlier onset of action after waking in the morning than those forms currently available and which have a sustained therapeutic effect.

U.S. Publication No. 2008/0182816 describes certain polysaccharide prodrugs.

### SUMMARY OF THE INVENTION

The present inventors have discovered amphetamine prodrugs which release amphetamine in the colon, for example, upon cleavage by colonic microflora. Without wishing to be bound by any particular theory, the prodrugs do not release substantial quantities of amphetamine outside the presence of certain bacterial enzymes and, therefore, are resistant to abuse by parenteral routes of administration, such as intravenous “shooting”, intranasal “snorting”, and inhalation “smoking”. Following oral administration, the prodrugs are poorly absorbed in the upper gastrointestinal tract due to their physicochemical properties and a large proportion of the dose is delivered to the colon. Furthermore, because there is a significant lag time until the prodrugs reach the colon (approximately 5 hours in man) and release the amphetamine, the prodrugs when administered orally do not provide an immediate effect. The prodrugs also release the amphetamine over an extended period of time due to the action of the colonic microflora, and thus can provide a sustained therapeutic effect with a diminished or reduced euphoric effect. The prodrugs have properties beneficial for once-daily administration at night (before bedtime) and for maintaining efficacious levels of amphetamine in a subject.

One embodiment of the invention is an amphetamine prodrug or an oral dosage form containing an amphetamine prodrug which, when orally administered, releases the amphetamine in the colon of a subject. The amphetamine in the prodrug is covalently bound to a chemical moiety (such as a sugar acid or an amino-saccharide) in a manner that diminishes or eliminates pharmacological activity of amphetamine until released. The amphetamine may be released by colonic microflora such as bacterial enzymes in the colon (e.g., amidases, azoreductases, and/or glucosidases). Preferably, the prodrug does not provide substantial release of the amphetamine in the gastrointestinal tract prior to reaching the colon. In one embodiment, the amphetamine prodrug, while conjugated, is substantially not absorbed by a subject upon oral administration. Preferably, the prodrugs are stable in tests that simulate procedures likely to be used by illicit chemists in attempts to release the amphetamine. Furthermore, preferably, after release of the amphetamine, the remaining compound which is derived from the chemical moiety is a naturally occurring compound (e.g., a non-toxic naturally occurring compound).

In one preferred embodiment, the amphetamine prodrug is a conjugate of amphetamine with a naturally occurring sugar acid or a naturally occurring amino-saccharide. In one embodiment, the sugar moiety (in open chain or closed ring form) in the sugar acid is separated from the amino terminus of the amphetamine by at least two atoms. Alternatively, the amphetamine prodrug may be conjugated to an unnatural sugar acid or unnatural amino-saccharide.

Because the amphetamine prodrugs may require bacterial enzymes to release the amphetamine, no or only limited amounts of amphetamine are released by the prodrugs following parenteral administration, such as by intravenous or intranasal administration or by inhalation.

Yet another embodiment is an amphetamine prodrug comprising amphetamine conjugated at its amino terminus (i) to the acid group of a sugar acid to form an amide linkage, or (ii) to an amino group of an amino-saccharide to form a hydrazine linkage, wherein (a) the sugar acid is not gulonic acid and (b) optionally the sugar moiety (in open chain or closed ring form) in the sugar acid is separated from the amino terminus of the amphetamine by at least two atoms. Preferably, the amphetamine prodrug releases the amphetamine upon cleavage in the colon of a subject. In one embodiment, the amphetamine prodrug releases the amphetamine upon cleavage by bacterial enzymes (e.g., amidases, azoreductases, and/or glucosidases) in the colon of a subject. For example, the amphetamine prodrug may be cleaved by bacterial azoreduction to release the amphetamine. Preferably, after release of the amphetamine, the resulting sugar-containing compound is a naturally occurring compound.

In one embodiment, the amphetamine prodrug has the formula (I):

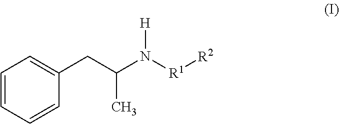

or is a tautomer thereof, or a pharmaceutically acceptable salt thereof, wherein
R¹ is —C(O)— or —NH—; and
R¹, R² is a sugar acid or aminosaccharide,
   where (i) the acid moiety of the sugar acid is conjugated to the amino group of the amphetamine to form an amide linkage, (ii) the sugar acid is not gulonic acid and the prodrug is not a metabolite of amphetamine (such as amphetamine N-glucoronide) and (iii) optionally the sugar moiety (in open chain or closed ring form) in the sugar acid is separated from the amino terminus of the amphetamine by at least two atoms. Preferably, when —R¹-R² is a sugar acid, the acid moiety of the sugar acid is conjugated to the amino group of the amphetamine to form an amide linkage. Preferably, when —R¹-R² is an amino-saccharide, the amino moiety of the amino-saccharide is conjugated to the amino group of the amphetamine to form a hydrazine (—NH—NH—) linkage.

In a preferred embodiment, the compound has the formula (IA):

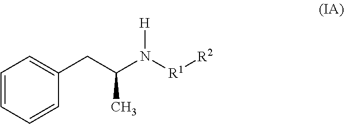

or is a tautomer thereof, or a pharmaceutically acceptable salt thereof, wherein R¹ and R² are defined as in formula (I).

In one embodiment, R¹ in formula (I) or (IA) is —C(O)—, and —R¹-R² is a sugar acid. The sugar acid may be conjugated through its acid group to the amino terminus of the amphetamine to form an amide bond. In one preferred embodiment, the sugar acid, has a substituent —NR³C(O)R⁴ at the alpha position adjacent to the aldehyde group (as it exists in its open form). R³ is hydrogen or C₁-C₄ alkyl, and R⁴ is C₁-C₄ alkyl (e.g., methyl, ethyl, n-propyl, and n-butyl). In one embodiment, R⁴ is a straight chain C₁-C₄ alkyl. In one more preferred embodiment, R³ is hydrogen and R⁴ is methyl. In another embodiment, R³ is hydrogen and R⁴ is ethyl.

In one embodiment, R¹ in formula (I) or (IA) is —NH—, and —R¹-R² is an amino-saccharide, such as an amino-monosaccharide. The amino-saccharide may be conjugated through its amino group to the amino terminus of the amphetamine to form a hydrazine bond.

In yet another embodiment, the compound has the formula (IB):

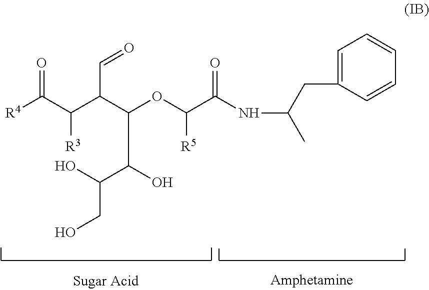

or is a tautomer thereof, or a pharmaceutically acceptable salt thereof, wherein R³ and R⁴ are as defined above, and R⁵ is hydrogen or C₁-C₄ alkyl (e.g., methyl). In one embodiment, R⁵ is hydrogen. In another embodiment, R⁵ is methyl.

In one preferred embodiment, R³ in the compound of formula (IB) is hydrogen.

In one preferred embodiment, R⁴ in the compound of formula (IB) is methyl.

In one preferred embodiment, R⁴ in the compound of formula (IB) is ethyl.

In yet another embodiment, the compound has the formula (IC):

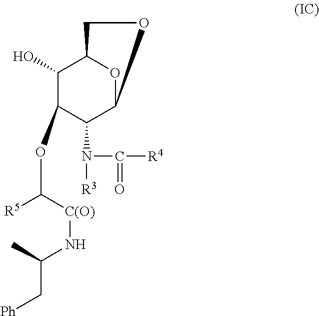

or is a tautomer thereof, or a pharmaceutically acceptable salt thereof, wherein R³ and R⁴ are as defined above, and R⁵ is hydrogen or C₁-C₄ alkyl (e.g., methyl). In one embodiment, R⁵ is hydrogen. In another embodiment, R⁵ is methyl.

In one preferred embodiment, R³ in the compound of formula (IC) is hydrogen.

In one preferred embodiment, R⁴ in the compound of formula (IC) is methyl.

In one preferred embodiment, R⁴ in the compound of formula (IC) is ethyl.

In yet another embodiment, the compound has the formula (ID):

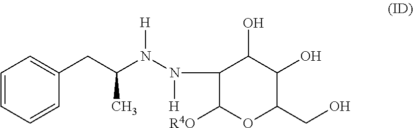

or is a tautomer thereof, or a pharmaceutically acceptable salt thereof, wherein R⁴ is C₁-C₈ alkyl (e.g., methyl, ethyl, n-propyl, isopropyl, n-butyl, t-butyl, and n-octyl), such as a straight chain C₁-C₈ alkyl. In one embodiment, R⁴ is C₁-C₄ alkyl, such as a straight chain C₁-C₄ alkyl. In one embodiment, R⁴ is methyl. In another embodiment, R⁴ is ethyl. In yet another embodiment, R⁴ is n-octyl. In a preferred embodiment, the sugar group in formula (ID) is a D-sugar.

One or more functional groups on the sugar acid or amino-saccharide may be protected. For example, an amino group on the amino-saccharide or the sugar acid may be protected, e.g., with an acetyl moiety. One or more hydroxyl groups on the amino-saccharide or the sugar acid may be protected, e.g., with C₁-C₁₀ alkyl (e.g., methyl, ethyl, propyl, or n-octyl).

Preferably, the sugar acid or amino-saccharide in the prodrug is a naturally occurring sugar acid or naturally occurring amino-saccharide.

Preferably, after release of the amphetamine by the amphetamine prodrug, the resulting sugar-containing compound is a naturally occurring compound.

Representative examples of the amphetamine prodrugs containing a sugar acid or amino-saccharide include, but are not limited to, the compounds listed below and their tautomers and pharmaceutically acceptable salts.

| | |
|---|---|
| 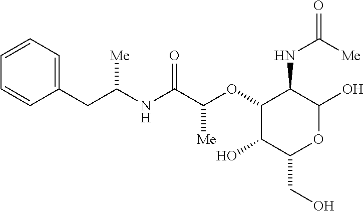 | Compound 1 |
| (2R)-2-(((3R,4R,5R,6R)-3-acetamido-2,5-dihydroxy-6-(hydroxymethyl)tetrahydro-2H- | |
| pyran-4-yl)oxy)-N-((S)-1-phenylpropan-2-yl)propanamide | |
| 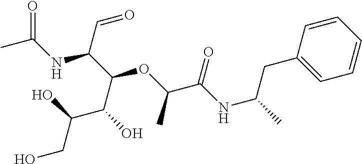 [SMILES: CC(=O)N[C@@H](C=O)[C@@H](O[C@H](C)C(=O)N[C@@H](C)Cc1ccccc1)[C@H](O)[C@H](O)CO] | Compound 1 |
| (R)-2-(((2R,3R,4R,5R)-2-acetamido-4,5,6-trihydroxy-1-oxohexan-3-yl)oxy)-N-((S)-1- | |
| phenylpropan-2-yl)propanamide | |
| 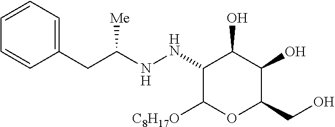 | Compound 2 |
| (2R,3R,4R,5R)-2-(hydroxymethyl)-6-(octyloxy)-5-(2-((S)-1-phenylpropan-2-yl) | |
| hydrazinyl)tetrahydro-2H-pyran-3,4-diol | |
| 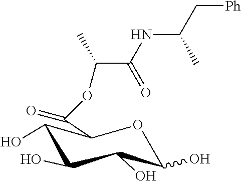 | Compound 3 |
| (2S,3S,4S,5R)-(R)-1-oxo-1-(((S)-1-phenylpropan-2-yl)amino)propan-2-yl 3,4,5,6- | |
| tetrahydroxytetrahydro-2H-pyran-2-carboxylate | |
| 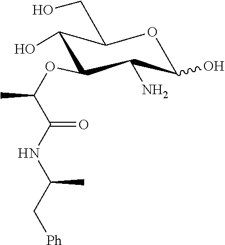 | Compound 4 |
| (2R)-2-(((3R,4R,5S,6R)-3-amino-2,5-dihydroxy-6-(hydroxymethyl)tetrahydro-2H-pyran-4- | |
| yl)oxy)-N-((S)-1-phenylpropan-2-yl)propanamide | |
| 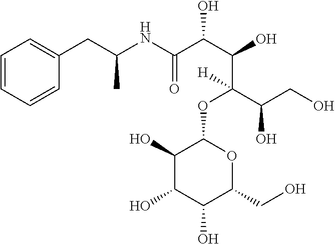 [SMILES: C[C@@H](Cc1ccccc1)NC(=O)[C@H](O)[C@@H](O)[C@H](O[C@@H]1O[C@H](CO)[C@H](O)[C@H](O)[C@H]1O)[C@H](O)CO] | Compound 5 |
| (2R,3R,4R,5R)-2,3,5,6-tetrahydroxy-N-((S)-1-phenylpropan-2-yl)-4-(((2S,3R,4S,5R,6R)- | |
| 3,4,5-trihydroxy-6-(hydroxymethyl)tetrahydro-2H-pyran-2-yl)oxy)hexanamide | |
| 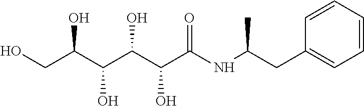 [SMILES: C[C@@H](Cc1ccccc1)NC(=O)[C@H](O)[C@@H](O)[C@H](O)[C@H](O)CO] | Compound 6 |
| (2R,3S,4R,5R)-2,3,4,5,6-pentahydroxy-N-((S)-1-phenylpropan-2-yl)hexanamide | |
| 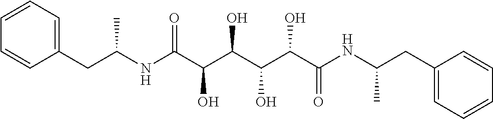 [SMILES: C[C@@H](Cc1ccccc1)NC(=O)[C@@H](O)[C@H](O)[C@H](O)[C@@H](O)C(=O)N[C@@H](C)Cc1ccccc1] | Compound 7 |
| (2R,3S,4R,5S)-2,3,4,5-Tetrahydroxy-N1,N6-bis((S)-1-phenylpropan-2-yl)hexanediamide | |
| 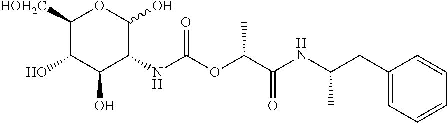 [SMILES: C[C@@H](Cc1ccccc1)NC(=O)[C@@H](C)OC(=O)N[C@H]1C(O)O[C@H](CO)[C@@H](O)[C@@H]1O] | Compound 8 |
| (1R)-1-{[(2S)-1-Phenylpropan-2-yl]carbamoyl}ethyl N-[(3R,4R,5S,6R)-2,4,5-trihydroxy-6- | |
| (hydroxymethyl)oxan-3-yl]carbamate | |
| 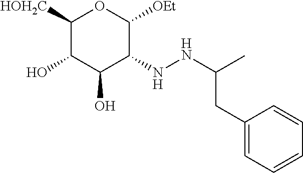 [SMILES: CCO[C@H]1O[C@H](CO)[C@@H](O)[C@H](O)[C@H]1NNC(C)Cc1ccccc1] | Compound 9 |
| (2R,3S,4R,5R,6S)-6-Ethoxy-2-(hydroxymethyl)-5-[2-(1-phenylpropan-2-yl)hydrazin-1- | |
| yl]oxane-3,4-diol | |
| 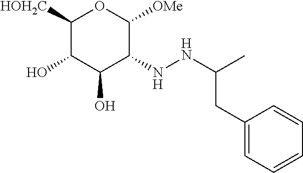 [SMILES: CO[C@H]1O[C@H](CO)[C@@H](O)[C@H](O)[C@H]1NNC(C)Cc1ccccc1] | Compound 10 |
| (2R,3S,4R,5R,6S)-6-Methoxy-2-(hydroxymethyl)-5-[2-(1-phenylpropan-2-yl)hydrazin-1- | |
| yl]oxane-3,4-diol | |
| 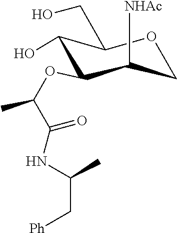 | Compound 11 |
| (R)-2-(((2R,3S,4R,5R)-5-Acetamido-3-hydroxy-2-(hydroxymethyl)tetrahydro-2H-pyran-4- | |
| yl)oxy)-N-((S)-1-phenylpropan-2-yl)propanamide | |
| 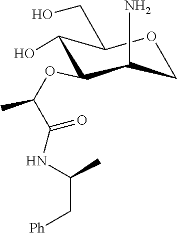 | Compound 12 |
| (R)-2-(((2R,3S,4R,5R)-5-Amino-3-hydroxy-2-(hydroxymethyl)tetrahydro-2H-pyran-4- | |
| yl)oxy)-N-((S)-1-phenylpropan-2-yl)propanamide | |
| 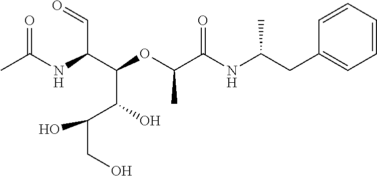 [SMILES: CC(=O)N[C@@H](C=O)[C@@H](O[C@H](C)C(=O)N[C@H](C)Cc1ccccc1)[C@H](O)[C@H](O)CO] | Compound 13 |
| (2R)-2-{[(2R,3R,4R,5R)-2-Acetamido-4,5,6-trihydroxy-1-oxohexan-3-yl]oxy}-N-[(2R)-1- | |
| phenylpropan-2-yl]propanamide | |
| 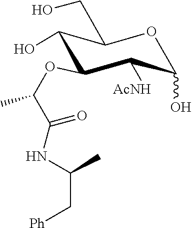 | Compound 14 |
| (2S)-2-(((3R,4R,5S,6R)-3-Acetamido-2,5-dihydroxy-6-(hydroxymethyl)tetrahydro-2H-pyran- | |
| 4-yl)oxy)-N-((S)-1-phenylpropan-2-yl)propanamide | |
| 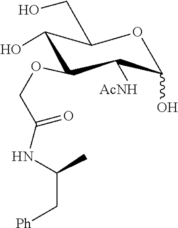 | Compound 15 |
| 2-(((3R,4R,5S,6R)-3-acetamido-2,5-dihydroxy-6-(hydroxymethyl)tetrahydro-2H-pyran-4- | |
| yl)oxy)-N-((S)-1-phenylpropan-2-yl)acetamide | |
| 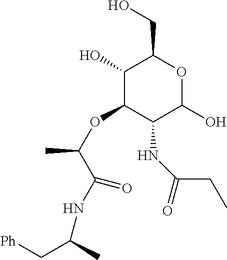 [SMILES: CCC(=O)N[C@H]1C(O)O[C@H](CO)[C@@H](O)[C@@H]1O[C@H](C)C(=O)N[C@@H](C)Cc1ccccc1] | Compound 16 |
| (2R)-2-[(3R,4R,5S)-2,5-dihydroxy-6-(hydroxymethyl)-3-(propanoylamino)tetrahydro pyran- | |
| 4-yl]oxy-N-[(1S)-1-methyl-2-phenyl-ethyl]propanamide | |
| 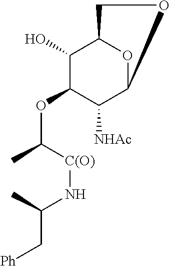 | Compound 17 |
| (2R)-2-[[(2S,3R,4R)-4-acetamido-2-hydroxy-6,8-dioxabicyclo[3.2.1]octan-3-yl]oxy]-N-[(1R)- | |
| 1-methyl-2-phenyl-ethyl]propanamide | |
| 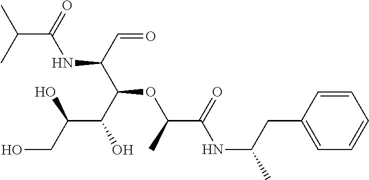 [SMILES: CC(C)C(=O)N[C@@H](C=O)[C@@H](O[C@H](C)C(=O)N[C@@H](C)Cc1ccccc1)[C@H](O)[C@H](O)CO] | Compound 18 |
| N-((2R,3R,4R,5R)-4,5,6-trihydroxy-1-oxo-3-(((R)-1-oxo-1-(((S)-1-phenylpropan-2- | |
| yl)amino)propan-2-yl)oxy)hexan-2-yl)isobutyramide | |
| 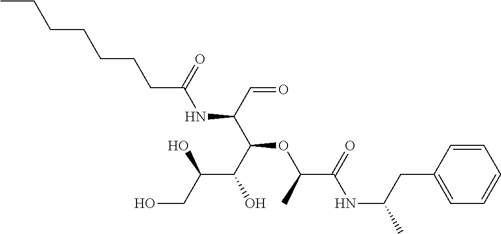 [SMILES: CCCCCCCC(=O)N[C@@H](C=O)[C@@H](O[C@H](C)C(=O)N[C@@H](C)Cc1ccccc1)[C@H](O)[C@H](O)CO] | Compound 19 |
| N-((2R,3R,4R,5R)-4,5,6-trihydroxy-1-oxo-3-(((R)-1-oxo-1-(((S)-1-phenylpropan-2- | |
| yl)amino)propan-2-yl)oxy)hexan-2-yl)octanamide | |

The amphetamine in the prodrugs of the present invention (including in formulas (I), (IA), and (IB)) can be, for example, racemic amphetamine, l-amphetamine or d-amphetamine. In a preferred embodiment, the amphetamine in the prodrug is d-amphetamine.

In one preferred embodiment, the sugar group of the sugar acid or amino-saccharide is a D-sugar. In another embodiment, the sugar group of the sugar acid or amino-saccharide is an L-sugar. In yet another embodiment, all of the stereo-centers in the sugar group of the sugar acid or amino-saccharide are of the R-configuration. In yet another embodiment, all of the stereo-centers in the sugar acid (including, but not limited to, the sugar group) are of the R-configuration. In yet another embodiment, all of the stereo-centers in the sugar acid (including, but not limited to, the sugar group), except for the 2- or 5-position, are of the R-configuration, and the stereocenter at the 2- or 5-position is in the S configuration.

The amphetamine prodrugs of the present invention preferably release the amphetamine upon cleavage by bacterial enzymes (e.g., amidases, azoreductases, and/or glucosidases) in the colon of a subject (e.g., human subject). In one embodiment, the amphetamine prodrug predominantly or exclusively release the amphetamine upon cleavage by bacterial enzymes in the colon of a subject.

In one embodiment, the amphetamine prodrugs which have a hydrazine linkage, are cleaved by bacterial azoreduction to release the amphetamine.

Preferably, the amphetamine prodrug does not release substantial quantities of amphetamine upon oral administration to a subject until reaching the colon. The amphetamine prodrug preferably also does not release substantial quantities of amphetamine until at least 1, 2, 3, 4, or 5 hours after oral administration to a subject.

In one embodiment, the amphetamine prodrug is substantially not absorbed by a subject.

In another embodiment, the amphetamine prodrug or a pharmaceutical composition containing it does not release substantial quantities of amphetamine upon intravenous or intranasal administration to a subject. For example, the release of amphetamine is diminished or eliminated when the prodrug or pharmaceutical composition is administered by parenteral routes.

Preferably, the amphetamine prodrug or a pharmaceutical composition containing it have no or a substantially decreased pharmacological activity when administered through injection or intranasal routes of administration.

The amphetamine prodrug of the present invention may be incorporated into a pharmaceutical composition, such as an oral dosage form (e.g., an immediate release or controlled release dosage form). In one embodiment, the pharmaceutical composition includes an amphetamine prodrug of the present invention and at least one excipient. In one preferred embodiment, the amphetamine prodrug is incorporated into an oral immediate release dosage form. In another preferred embodiment, the amphetamine prodrug is incorporated into an oral delayed release dosage form, such that the amphetamine prodrug is not released until exiting the stomach or until entering the colon. The dosage form may, for instance, have an enteric coating and/or be a gastroretentive formulation. In another embodiment, the oral dosage form may be a sustained release dosage form or a delayed and sustained release dosage form. In one embodiment, the delayed release dosage form begins releasing the amphetamine prodrug after about 6 to about 10 hours, and preferably after about 8 to about 10 hours.

In one embodiment, the amphetamine prodrug or oral dosage form containing the amphetamine prodrug, upon oral administration, results in delayed release of amphetamine for at least 6, 7, or 8 hours following administration. In one preferred embodiment, the amphetamine prodrug or oral dosage form containing the amphetamine prodrug, upon oral administration, begins to provide significant release of the amphetamine from about 7 to about 10 hours after administration. In a more preferred embodiment, significant release of the amphetamine begins to occur from about 8 to about 9 hours after administration. This delayed release provides convenience of dosing as well as abuse resistance.

In one preferred embodiment, the amphetamine prodrug or oral dosage form containing the amphetamine prodrug is orally administered in the evening, for example, prior to going to bed (such as within 30 minutes of going to bed). This provides convenience of dosing, especially in children who may be difficult to dose in the morning.

In one embodiment, the amphetamine prodrug or oral dosage form containing the amphetamine prodrug provides a Tₘₐₓ of the amphetamine released in a subject of from about 5 to about 16 hours. In another embodiment, the Tₘₐₓ ranges from about 6 to about 10 hours.

In another embodiment, the amphetamine prodrug or oral dosage form containing the amphetamine prodrug does not release substantial quantities of amphetamine until at least one hour after oral administration to a subject. Preferably, substantial quantities of the amphetamine are not released at least until after 2, 3, 4, 5, 6, 7, 8, 9 or 10 hours after oral administration. In one embodiment, the amphetamine prodrug does not release substantial quantities of amphetamine until at least three hours after oral administration to a subject.

In one embodiment, no further sustained release additives are required in the oral dosage form to achieve a blunted or reduced pharmacokinetic curve (e.g., reduced euphoric effect) while achieving therapeutically effective amounts of amphetamine release when taken orally.

In another embodiment, the pharmaceutical composition contains an amount of the amphetamine prodrug sufficient to provide a therapeutically bioequivalent area under the curve (AUC) when compared to amphetamine alone, but does not provide a Cₘₐₓ spike or an equivalent Cₘₐₓ.

In one embodiment, the oral dosage form of the present invention maintains its effectiveness and abuse resistance following the crushing of the oral dosage form (e.g., tablet or capsule) used to deliver the therapeutic component.

The amphetamine prodrug reduces the rate of absorption of the amphetamine, and the toxicity of the amphetamine, as compared to delivering the unconjugated amphetamine. The amphetamine prodrug also does not cause blood level spiking.

Yet another embodiment is a method of treating in a subject a disorder treatable with an amphetamine. The method includes orally administering to the subject an effective amount of an amphetamine prodrug or pharmaceutical composition of the present invention. Preferably, upon oral administration, the amphetamine released by the prodrug is predominantly or exclusively released in the colon. The disorder may be, for example, attention deficit hyperactivity disorder (ADHD), obesity, binge eating disorder, negative symptoms of schizophrenia, narcolepsy, appetite suppression, depression, anxiety, withdrawals (e.g., alcohol withdrawals or drug withdrawals), or wakefulness. These disorders can be treated with reduced or prevented abuse potential. The disorder may also be stimulant (e.g., cocaine or methamphetamine) abuse and/or addiction. The amphetamine prodrug or pharmaceutical composition of the present invention may also be administered to improve battle field alertness and to combat fatigue.

A preferred embodiment is a method of treating ADHD in a subject comprising administering (for instance, orally) an effective amount of an amphetamine prodrug or pharmaceutical composition of the present invention. The subject may be a human, such as an adult or child (e.g., adolescent).

In one embodiment, upon oral administration of the amphetamine prodrug or pharmaceutical composition containing it, the blood levels of amphetamine in the subject are not unnecessarily elevated (i.e., blood level spikes) thus preventing additional cardiovascular stress through, for example, increased blood pressure and/or heart rate.

In another embodiment, upon oral administration of the amphetamine prodrug or pharmaceutical composition containing it, the blood levels of amphetamine in the patient can maintain a therapeutically effect level, but do not result in an euphoric effect.

In yet another embodiment, upon oral administration, the amphetamine prodrug or pharmaceutical composition containing it provide the subject with a therapeutically effective amount of the amphetamine prodrug, which can provide a therapeutically bioequivalent area under the curve (AUC) when compared to free amphetamine, but does not provide a maximum plasma concentration (Cₘₐₓ) which results in an increased heart rate, increased blood pressure or drug related euphoria when taken orally.

The amphetamine prodrug and pharmaceutical compositions containing it can provide a steady-state plasma release curve for amphetamine which can provide a therapeutically effective bioavailability but prevent spiking or increased plasma concentrations compared to unconjugated amphetamine.

Another embodiment is a method for diminishing or reducing behavioral deterioration or the rebound effect caused by amphetamines by orally administering to a subject an amphetamine prodrug or a pharmaceutical composition of the present invention.

Another embodiment is a method for reducing or preventing abuse of an amphetamine comprising providing, administering, or prescribing an amphetamine prodrug or pharmaceutical composition of the present invention to a subject in need thereof, wherein the pharmacological activity of the amphetamine is decreased (preferably substantially decreased) when the amphetamine prodrug or pharmaceutical composition is used in a manner inconsistent with the manufacturer's instructions.

Another embodiment is a method of diminishing or reducing behavioral deterioration or the rebound effect of amphetamine treatment comprising providing, administering, or prescribing an amphetamine prodrug or pharmaceutical composition of the present invention to a subject in need thereof, wherein the amphetamine prodrug or pharmaceutical composition can diminish or reduce the potential of behavioral deterioration or the rebound effect from amphetamine treatment.

Yet another embodiment is a method for reducing or preventing the euphoric effect of an amphetamine comprising providing, administering, or prescribing to a subject in need thereof, an amphetamine prodrug or pharmaceutical composition of the present invention that can decrease the pharmacological activity of the amphetamine when the prodrug or pharmaceutical composition is used in a manner inconsistent with the manufacturer's instructions.

### BRIEF DESCRIPTION OF THE DRAWINGS

FIG. 1 is a graph showing the average amount of amphetamine in the blood plasma of two male dogs following oral administration of compound 1 (0.5 mg/kg) over time.

FIG. 2 is a graph showing the amount of amphetamine in the blood plasma of a cynomolgus monkey following oral administration of compound 1 (0.5 mg/kg) over time.

FIG. 3 is a graph showing the amount of amphetamine in the blood plasma of a cynomolgus monkey following intravenous administration of compound 1 (0.2 mg/kg) over time.

FIG. 4 is a graph showing the amount of amphetamine in the blood plasma of male dogs over time following oral administration of d-amphetamine or compound 1 and intranasal administration of compound 1 as described in Example 6.

FIG. 5 is a graph showing the release of amphetamine following incubation of compound 1 in human fecal suspensions from three subjects as described in Example 8.

### DETAILED DESCRIPTION OF THE INVENTION

### Definitions

The term “amphetamine”, unless otherwise indicated, refers to any of the sympathomimetic phenethylamine derivatives which have central nervous system stimulant activity and a free amino group (—NH₂), such as but not limited to, amphetamine (alpha-methyl-phenethylamine), methamphetamine, and 3,4-methylenedioxyamphetamine. The amphetamine can be racemic amphetamine, l-amphetamine or d-amphetamine. A preferred amphetamine is d-amphetamine.

As used herein, “in a manner inconsistent with the manufacturer's instructions” or similar expression is meant to include, but is not limited to, consuming amounts greater than amounts described on the drug label or ordered by a licensed physician, and/or altering by any means (e.g., crushing, breaking, melting, or separating) the dosage form such that the composition may be injected, inhaled or smoked.

As used herein, the phrases such as “decreased,” “reduced,” “diminished” or “lowered” is meant to include at least a 10% change in pharmacological activity with greater percentage changes being preferred for reduction in abuse potential and overdose potential. For instance, the change may also be greater than 25%, 35%, 45%, 55%, 65%, 75%, 85%, 95%, 96%, 97%, 98%, 99%, or increments therein.

Unless otherwise specified, the term “naturally occurring” refers to occurring in nature, for example, in bacteria or in a mammal (e.g., a human).

The term “pharmaceutically acceptable salts” embraces salts commonly used to form alkali metal salts and to form addition salts of free acids or free bases. The nature of the salt is not critical, provided that it is pharmaceutically acceptable. Suitable pharmaceutically acceptable acid addition salts may be prepared from an inorganic acid or an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic (e.g., trifluoroacetic acid), propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, β-hydroxybutyric, salicylic, galactaric and galacturonic acid. Suitable pharmaceutically acceptable base addition salts include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. These salts may be prepared, for example, by reacting, in another embodiment, the appropriate acid or base with the compound.

In one embodiment, the term “pharmaceutically acceptable carriers” includes, but is not limited to, 0.01-0.1M and preferably 0.05M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In one embodiment the level of phosphate buffer used as a pharmaceutically acceptable carrier is between about 0.01 to about 0.1M, or between about 0.01 to about 0.09M in another embodiment, or between about 0.01 to about 0.08M in another embodiment, or between about 0.01 to about 0.07M in another embodiment, or between about 0.01 to about 0.06M in another embodiment, or between about 0.01 to about 0.05M in another embodiment, or between about 0.01 to about 0.04M in another embodiment, or between about 0.01 to about 0.03M in another embodiment, or between about 0.01 to about 0.02M in another embodiment, or between about 0.01 to about 0.015 in another embodiment.

The term “prodrug”, as used herein, generally refers to a compound, which is pharmaceutically acceptable and upon administration is converted to a desired active compound, here amphetamine. The prodrug may be therapeutically inactive until cleaved to release the active compound.

The term “subject” refers to a mammal, such as humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, and cats, avian species, such as chickens, turkeys, and songbirds. Preferably, the subject is a human. The subject can be, for example, a child, such as an adolescent, or an adult.

The term “treatment” refers to any treatment of a pathologic condition in a mammal, particularly a human, and includes: (i) preventing the pathologic condition from occurring in a subject which may be predisposed to the condition but has not yet been diagnosed with the condition and, accordingly, the treatment constitutes prophylactic treatment for the disease condition; (ii) inhibiting the pathologic condition, i.e., arresting its development; (iii) relieving the pathologic condition, i.e., causing regression of the pathologic condition; or (iv) relieving the conditions mediated by the pathologic condition.

The term “therapeutically effective amount” refers to that amount of a compound of the invention that is sufficient to effect treatment, as defined above, when administered to a mammal in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “pharmaceutically acceptable salts” includes, but is not limited to, salts well known to those skilled in the art, for example, mono-salts (e.g. alkali metal and ammonium salts) and poly salts (e.g. di- or tri-salts,) of the compounds of the invention. Pharmaceutically acceptable salts of compounds of formula I are where, for example, an exchangeable group, such as hydrogen in —OH or —NH— is replaced with a pharmaceutically acceptable cation (e.g. a sodium, potassium, or ammonium ion) and can be conveniently be prepared from a corresponding compound of formula I by, for example, reaction with a suitable base. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, .alpha.-ketoglutarate, and .alpha.-glycerophosphate. Suitable inorganic salts may also be formed, including hydrochloride, sulfate, nitrate, bicarbonate, and carbonate salts. Pharmaceutically acceptable salts may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example, calcium) salts of carboxylic acids can also be made.

### Amphetamine Prodrugs

It will be apparent to one skilled in the art that certain compounds of this invention may exist in tautomeric forms. All such tautomeric forms of the compounds are within the scope of the invention.

Unless otherwise specified, the sugar group in the amphetamine prodrug includes, but is not limited to, L- and D-sugars and natural sugars (including naturally occurring D-sugars), as well as any combination of any of the foregoing. In one preferred embodiment, the sugar group in the amphetamine prodrug is a D-sugar. The sugar group may also be in its anhydro form.

The present invention also includes the synthesis of all pharmaceutically acceptable isotopically-labelled compounds of the present invention (including those of Formulas (I), (IA), (IB), (IC), and (ID)) wherein one or more atoms are replaced by atoms having the same atomic number, but an atomic mass or mass number different from the atomic mass or mass number most commonly found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life and/or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds can generally be prepared by conventional techniques known to those skilled in the art or by processes analogous to those described using an appropriate isotopically-labelled reagent in place of the non-labelled reagent previously employed.

Sugar Acids

The sugar acid may be any tautomeric form, including in its open or cyclic (closed) form. It will be understood to those in the art that sugar groups often exist in an equilibrium between their open chain and cyclic forms. Hereto, the sugar groups in the sugar acids may be in their open or cyclic form. For instance, the sugar acid N-acetyl muramic acid (i.e., N-acetyl D-muramic acid) can exist in the forms shown below. (The structures below assume that the N-acetyl muramic acid is bound to amphetamine through its acid group.) Within the scope of the invention are compounds where the sugar acid is in its open form or cyclic form, or in equilibrium between the two forms. Where a sugar acid is shown in one form, it is intended to include the other tautomeric form as well as both open and closed forms in equilibrium unless otherwise specified.

The open sugar group or sugar in cyclic form is appropriately conjugated to permit bacteria and enzymes (e.g., amidases, azoreductases, and/or glucosidases) to cleave the amphetamine. In one embodiment, the open sugar group or sugar in cyclic form is at least one atom (e.g., one carbon atom) away from the amido or hydrazine linkage. In another embodiment, the sugar acid is acyclic.

The term “sugar acid” includes sugar molecules having an acid moiety. The sugar group in the sugar acid can be a monosaccharide, and optionally one or more of the hydroxyl groups in the sugar can be removed. The sugar acid can be naturally occurring, non-natural or synthetic, and unsubstituted or substituted (for example, with an amido group such as —NHC(O)CH₃). For example, in one embodiment, the sugar acid has an amido group of the formula —NR³C(O)R⁴ where R³ is hydrogen or C₁-C₄ alkyl, and R⁴ is C₁-C₄ alkyl (e.g., methyl, ethyl, n-propyl, and n-butyl). In one embodiment, R⁴ is a straight chain C₁-C₄ alkyl. In one more preferred embodiment, R³ is hydrogen and R⁴ is methyl. In another embodiment, R³ is hydrogen and R⁴ is ethyl. In one preferred embodiment, the sugar acid is one which one or more bacterial enzymes in the colon have an affinity, such as a sugar acid used to build cell walls in bacteria. The stereochemistry of the sugar acid is preferably that of a D-sugar.

Non-limiting examples of suitable sugar acids include N-acetyl muramic acid, anhydro N-acetyl muramic acid (1,6-anhydro N-acetyl D-muramic acid), muramic acid, lactobionic acid, glyceric acid, xylonic acid, gluconic acid, ketodeoxyoctulosonic acid (3-deoxy-d-manno-oct-2-ulosonic acid), galacturonic acid, tartaric acid, iduronic acid, galactonic acid (Mucic acid), glucaric acid and galonic acid. A preferred sugar acid is N-acetyl-D-muramic acid. Another example of a sugar acid is N-ketopropyl muramic acid (where the keto group is adjacent the nitrogen atom).

One or more of the functional groups on the sugar group may be protected. Suitable amino-protecting groups include, for example, acetyl. Suitable hydroxy-protecting groups include, for example, C₁-C₁₀ alkyl groups, such as methyl, ethyl, n-propyl, and octyl.

Amino-Saccharides

The amino saccharide may be in its open or cyclic (closed) form. The term “amino-saccharide” includes carbohydrate molecules where one or more hydroxy groups are replaced by an amino group. The sugar group in the amino-saccharide can be a monosaccharide, and optionally one or more of the hydroxyl groups in the sugar can be removed. In one preferred embodiment, the hydroxyl group at position 2 in the sugar is replaced by an amino group. The amino-saccharide may be a mono- or polysaccharide. The amino-saccharide can be naturally occurring, non-natural or synthetic, and unsubstituted or substituted (for example, with an acetyl (—NHC(O)CH₃) substituent on the amino group). In one preferred embodiment, the amino-saccharide is one which one or more bacterial enzymes in the colon have an affinity, such as an amino-saccharide used to build cell walls in bacteria. Non-limiting examples of suitable amino-saccharides include, but are not limited to, galactosamine, glucosamine, mannosamine, fucosamine, quinovosamine, lactosediamine, acosamine, bacillosamine, daunosamine, desosamine, forosamine, garosamine, kanosamine, kansosamine, mycaminose, mycosamine, perosamine, pneumosamine, purpurosamine, and rhodosamine. In one embodiment, the amino-saccharide includes a six-member sugar ring.

One or more of the functional groups on the sugar group may be protected. Suitable hydroxy-protecting groups include, for example, C₁-C₁₀ alkyl groups, such as methyl, ethyl, n-propyl, and octyl.

A preferred amino monosaccharide is C₁-octyl glucosamine.

### Pharmaceutical Compositions

The pharmaceutical composition may include one or more excipients including, but not limited to, lubricants (such as magnesium stearate, calcium stearate, zinc stearate, powdered stearic acid, hydrogenated vegetable oils, talc, polyethylene glycol, and mineral oil), colorants, binders (sucrose, lactose, gelatin, starch paste, acacia, tragacanth, povidone, polyethylene glycol, Pullulan and corn syrup), glidants (such as colloidal silicon dioxide and talc), surface active agents (such as sodium lauryl sulfate, dioctyl sodium sulfosuccinate, triethanolamine, polyoxyethylene sorbitan, poloxalkol, and quaternary ammonium salts), preservatives, stabilizers, adhesives (such as mucoadhesives), disintegrants, bulking substances, flavorings, sweeteners, pharmaceutically acceptable carriers, and other excipients (such as lactose, mannitol, glucose, fructose, xylose, galactose, sucrose, maltose, xylitol, sorbitol, chloride, sulfate and phosphate salts of potassium, sodium, and magnesium).

The amphetamine prodrugs may be formulated into an oral dosage forms (such as tablets and capsules) by methods known in the art. Examples of dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, troches, lozenges, chewable lozenges, beads, powders, granules, particles, microparticles, dispersible granules, cachets, thin strips, oral films, transdermal patches, and combinations thereof.

The pharmaceutical composition may be formulated to provide delayed and/or sustained release of the amphetamine prodrug.

In one embodiment, the pharmaceutical composition further comprise a polymer blend which comprises a hydrophilic polymer and/or a water-insoluble polymer. The polymers may be used to further enhance the sustained release/abuse resistant properties of the amphetamine prodrug. For instance, a composition might include: about 70% to about 100% amphetamine prodrug by weight, from about 0.01% to about 10% of a hydrophilic polymer (e.g., hydroxypropyl methylcellulose), from about 0.01% to about 2.5% of a water-insoluble polymer (e.g., acrylic resin), from about 0.01% to about 1.5% of additives (e.g., a lubricant such as magnesium stearate), and from about 0.01% to about 1% colorant by weight.

Hydrophilic polymers suitable for use in the sustained release formulations include one or more natural or partially or totally synthetic hydrophilic gums (such as acacia, gum tragacanth, locust bean gum, guar gum, and karaya gum), modified cellulosic substances (such as methylcellulose, hydroxymethylcellulose, hydroxypropyl methylcellulose, hydroxypropyl cellulose, hydroxyethylcellulose, and carboxymethylcellulose), proteinaceous substances (such as agar, pectin, carrageen, and alginates), and other hydrophilic polymers (such as carboxypolymethylene, gelatin, casein, zein, bentonite, magnesium aluminum silicate, polysaccharides, and modified starch derivatives). These hydrophilic polymers gel and dissolve slowly in aqueous acidic media thereby allowing the amphetamine prodrug to diffuse from the gel in the stomach. When the gel reaches the intestines it would dissolve in controlled quantities in the higher pH medium to allow further sustained release. Preferred hydrophilic polymers are the hydroxypropyl methylcelluloses such as those manufactured by The Dow Chemical Company and known as Methocel ethers, such as Methocel E1OM.

Formulations for oral administration can be presented as discrete units, such as capsules, caplets or tablets. These oral formulations also can comprise a solution or a suspension in an aqueous liquid or a non-aqueous liquid. The formulation can be an emulsion, such as an oil-in-water liquid emulsion or a water-in-oil liquid emulsion. The oils can be administered by adding the purified and sterilized liquids to a prepared enteral formula, which can then be placed in the feeding tube of a patient who is unable to swallow.

The preparation of delayed and/or sustained release forms of pharmaceutical compositions with the desired pharmacokinetic characteristics is known in the art and can be accomplished by a variety of methods. For example, oral controlled delivery systems include dissolution-controlled release (e.g., encapsulation dissolution control or matrix dissolution control), diffusion-controlled release (reservoir devices or matrix devices), ion exchange resins, osmotic controlled release or gastroretentive systems. Dissolution controlled release can be obtained, e.g., by slowing the dissolution rate of a drug in the gastrointestinal tract, incorporating the drug in an in soluble polymer, and coating drug particles or granules with polymeric materials of varying thickness. Diffusion controlled release can be obtained, e.g., by controlling diffusion through a polymeric membrane or a polymeric matrix. Osmotically controlled release can be obtained, e.g., by controlling solvent influx across a semipermeable membrane, which in turn carries the drug outside through a laser-drilled orifice. The osmotic and hydrostatic pressure differences on either side of the membrane govern fluid transport. Prolonged gastric retention may be achieved by, e.g., altering density of the formulations, bioadhesion to the stomach lining, or increasing floating time in the stomach. For further detail, see the Handbook of Pharmaceutical Controlled Release Technology, Wise, ed., Marcel Dekker, Inc., New York, N.Y. (2000), incorporated by reference herein in its entirety, e.g. Chapter 22 (“An Overview of Controlled Release Systems”).

In one embodiment, the pharmaceutical composition incorporates a gastroretentive system.

In one embodiment, the pharmaceutical composition includes a mucoadhesive to delay the passage of the composition through the gastrointestinal tract.

Tablets, capsules, and other forms of presentation provided in discrete units conveniently contain a daily dose, or an appropriate fraction thereof, of one or more of the prodrug compounds of the invention. For example, the units may contain from about 1 mg to about 1000 mg, alternatively from about 5 mg to about 500 mg, alternatively from about 5 mg to about 250 mg, alternatively from about 10 mg to about 100 mg of one or more of the prodrug of the present invention.

### Methods of Treatment

The amphetamine prodrugs and pharmaceutical compositions of the present invention can be administered to treat disorders treatable with an amphetamine, such as central nervous system (CNS) disorders. Non-limiting examples of disorders which can be treated include attention deficit hyperactivity disorder (ADHD), obesity, binge eating disorder, negative symptoms of schizophrenia, narcolepsy, appetite suppression, depression, anxiety, withdrawals (e.g., alcohol withdrawals or drug withdrawals), or wakefulness. These disorders can be treated with reduced or prevented abuse potential. The disorder may also be stimulant (e.g., cocaine, methamphetamine) abuse and/or addiction. The amphetamine prodrug or pharmaceutical composition of the present invention may also be administered to improve battle field alertness and to combat fatigue.

Typically, a therapeutically effective amount of the amphetamine prodrug or pharmaceutical composition is administered to treat the disorder.

The dose range for adult or pediatric human beings will depend on a number of factors including the age, weight and condition of the patient. Suitable oral dosages of the prodrugs of the present invention can be the molar equivalents of those typically found in treatments using that amphetamine. For example, dosages for amphetamine can range from about 1 mg to about 100 mg.

The amphetamine prodrug may be administered once-a-day, or two, three or more times a day. Preferably, the amphetamine prodrug is administered once-a-day.

### Methods of Synthesizing the Amphetamine Prodrugs

The compounds of the present invention can be prepared by the procedures set forth below. One skilled in the art will recognize that the methods described can be adapted to make similar compounds.

The conjugates of amphetamine and a sugar acid can be prepared by the scheme below (scheme I). In this scheme, a sugar acid or protected sugar acid (such as N-acetyl muramic acid) is directly reacted with amphetamine to form the conjugate. The compounds of Examples 1, 5, and 13 may be prepared by this method.

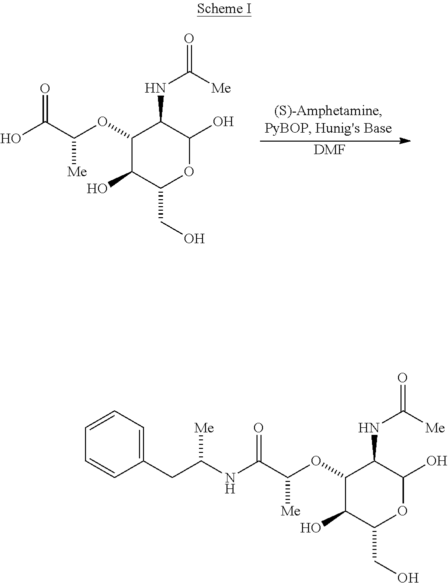

The conjugates of amphetamine and an amino-saccharide, such as the compounds of Examples 2, 9, and 10, can be prepared by schemes IA and IB below. The amino-saccharide may have one or more of its hydroxy groups protected (e.g., with a C₁-C₁₀ alkyl group) as exemplified in scheme IA below.

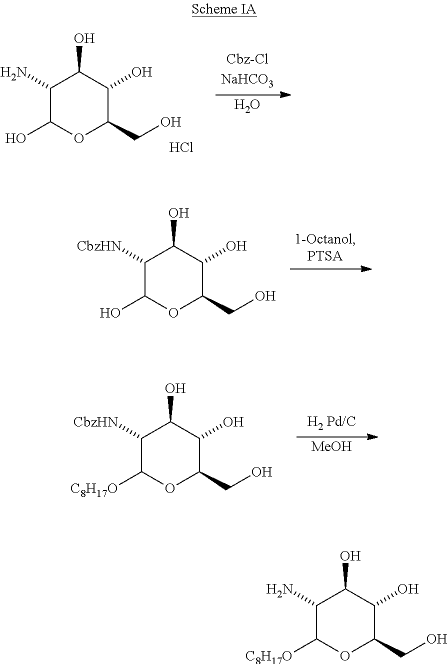

The protected amino-saccharide may be converted to its corresponding hydrazine, for example, using hydroxylamine-O-sulfonic acid. The hydrazine may then be reacted with phenylacetone to form a hydrazone conjugate. The hydrazone may then be reduced to form the final conjugate.

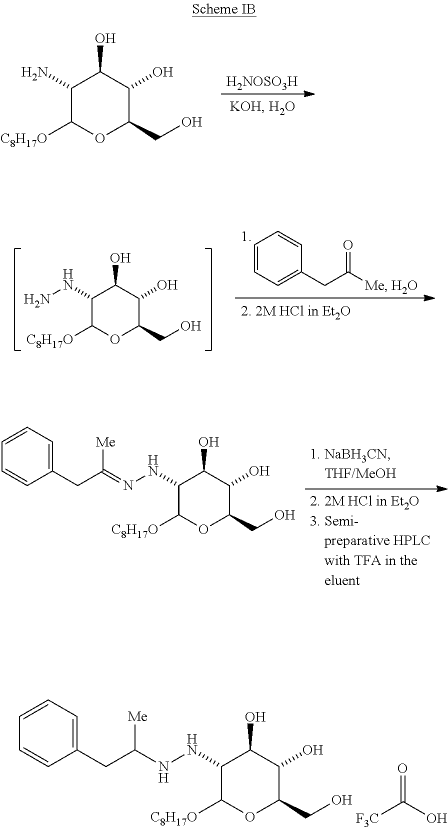

The compound of Example 14 ((S)-2-(((2R,3R,4R,5R)-2-acetamido-4,5,6-trihydroxy-1-oxohexan-3-yl)oxy)-N—((S)-1-phenylpropan-2-yl)propanamide) can be prepared by the process shown in Scheme IIA below.

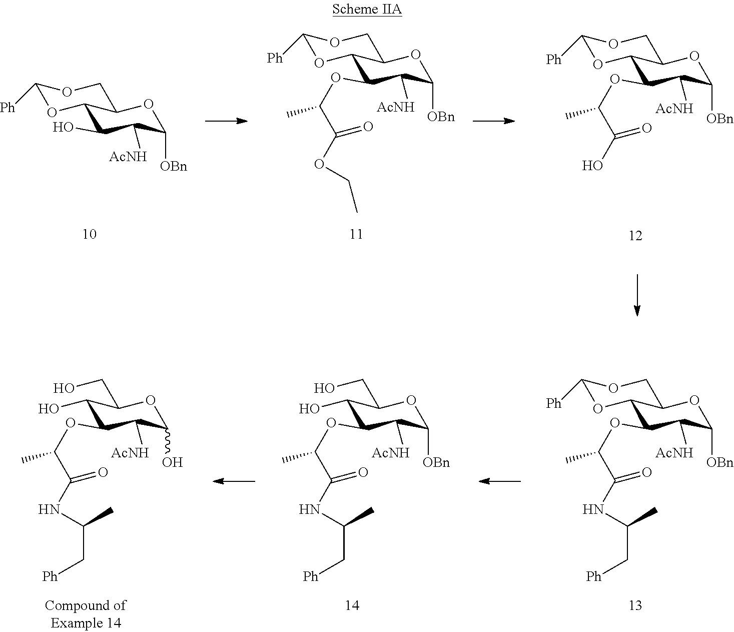

The dioxine compound 10 is converted into compound 11. The ethyl ester group of compound 11 is converted to a carboxylic acid group (e.g., by hydrolysis) to form compound 12. Compound 12 is converted to amphetamine conjugate 13, for example, by reaction with amphetamine (e.g., d-amphetamine). Compound 13 is deprotected, for example, by removing its dioxine protecting group (compound 14) and then its benzyl protecting group to yield the compound of Example 14.

The compound of Example 15 can be prepared in an analogous manner as shown by Scheme IIB below.

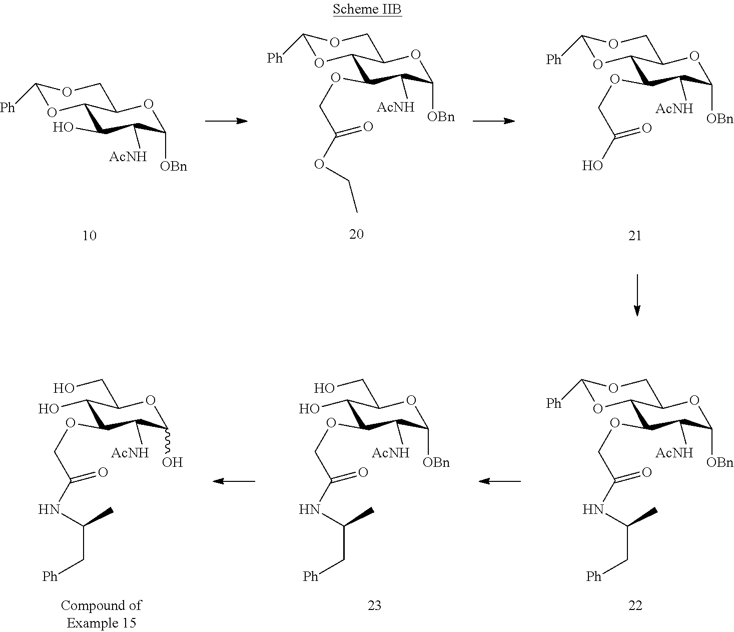

The compound of Example 4 can be prepared by a method analogous to that shown in Scheme IIB.

The compound of Example 11 can be prepared by the process shown in Scheme IIC below.

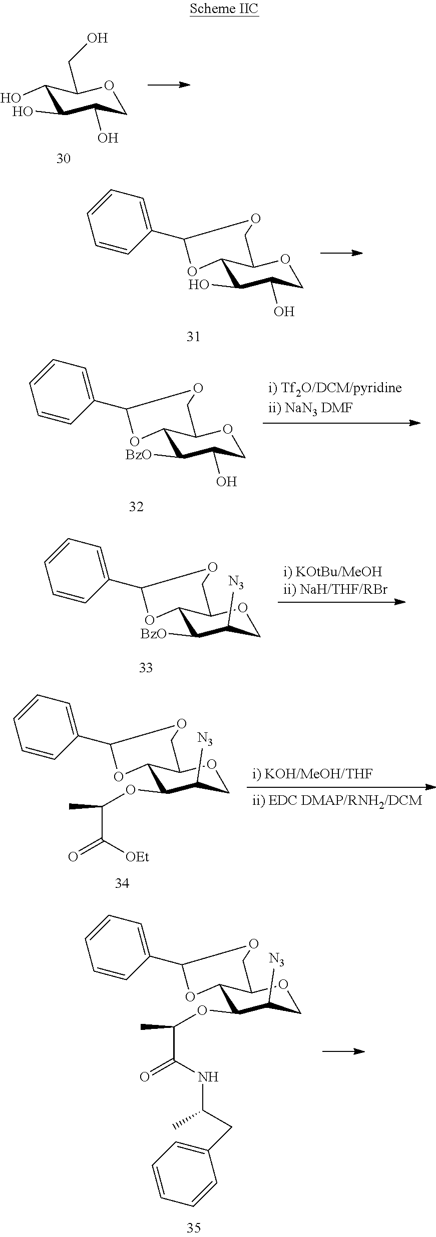

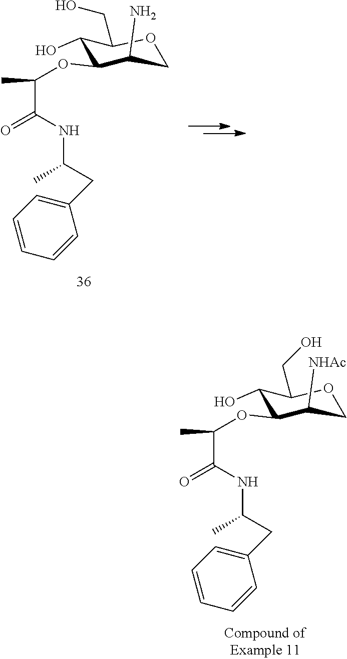

The hydroxyl groups at the 4- and 6-positions of sugar 30 are protected to form a dioxine intermediate 31. The hydroxyl group at the 3-position is then protected with a benzoyl group to yield intermediate 32. The hydroxyl group at the 2-position is converted to an azido group (—N₃), for example, by reaction with Tf₂O, dichloromethane (DCM), and pyridine, followed by treatment with sodium azide (NaN₃) in an appropriate solvent such as DMF. The benzoyl protecting group at the 3-position of the sugar is replaced to form the alkoxy group in intermediate 34, for example, by reacting potassium butoxide (KOtBu) in an appropriate solvent (such as methanol) followed by sodium hydride (NaH), THF, and RBr (where R is ethyl). The amphetamine is then conjugated to intermediate 34 at the ester group at the 3-position, for example, by reaction with potassium hydroxide (KOH), methanol and THF followed by EDC, DMAP, d-amphetamine (shown as RNH₂ in scheme IIC), and DCM. The dioxine protecting group is removed from intermediate 35 and the azide group is converted to an amino group to yield intermediate 36. The amino group at the 2-position of the sugar is then acetylated to yield the compound Example 11.

The compound of Example 12 can be prepared by a method analogous to that provided in Scheme IIC.

The compound of Example 3 can be prepared by the process shown in Scheme IID below.

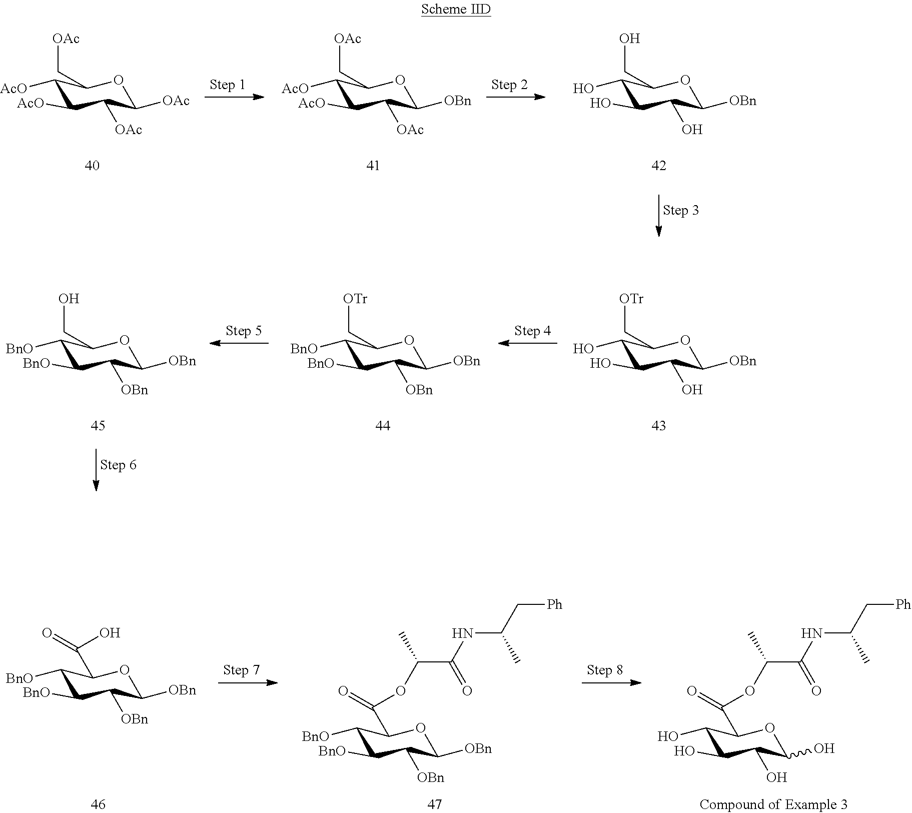

An acetoxy-protected sugar is reacted with benzyl alcohol (for example, in DCM and boron trifluoride diethyletherate) to selectively benzylate the sugar at the 1-position to yield compound 41. The compound is then deprotected at the other positions (i.e., de-acetylated, for example with methanol and sodium methoxide) to yield compound 42. Compound 42 is tritylated (e.g., with trityl chloride) at the 6-position (compound 43), and then benzylated to protect the other positions (compound 44). The 6-position is then deprotected (compound 45), for example, with acetic acid and trifluoroacetic acid, and oxidized, for example, with potassium dichromate, to yield compound 46. Compound 46 is reacted with (R)-2-hydroxy-N—((S)-1-phenylpropan-2-yl)propanamide to yield compound 47. The hydroxyl groups on the sugar ring of compound 47 are deprotected to yield the compound of Example 3.

The compound of Example 6 can be prepared by the process shown in Scheme IIE below.

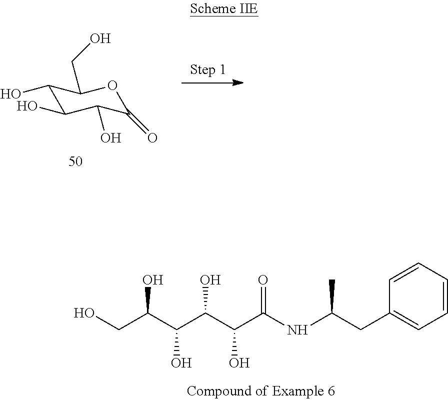 [SMILES: O=C1OC(CO)C(O)C(O)C1O ; C[C@@H](Cc1ccccc1)NC(=O)[C@H](O)[C@@H](O)[C@H](O)[C@H](O)CO]

The lactone 50 is reacted with (S)-amphetamine hemisulphate in the presence of a base, such as diisopropylethylamine, to yield the compound of Example 6.

The compound of Example 7 can be prepared by the process shown in Scheme IIF below.

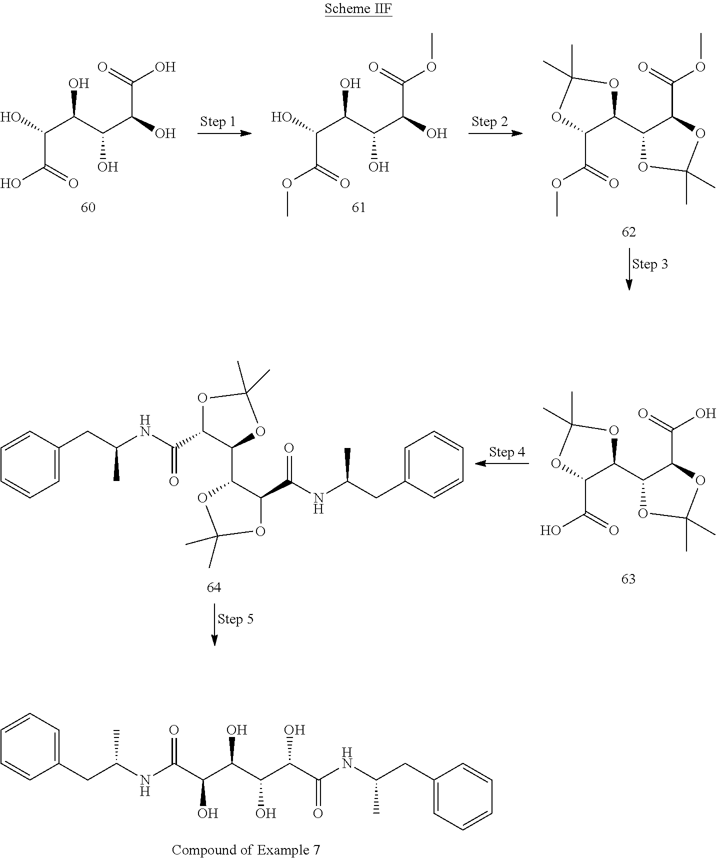

The dicarboxylic acid 60 is converted to dimethyl ester 61, for example, by reaction with methanol, e.g., in the presence of sulphuric acid. The hydroxyl substituents of compound 61 are protected by cyclizing each adjacent pair of hydroxyl substituents to form a dioxolane ring. For example, compound 61 can be reacted with 2,2-dimethoxypropane in the presence of an acid such as camphorsulfonic acid, to yield compound 62. The carboxylic acid groups of compound 62 are deprotected (compound 63), for example, by reaction with sodium hydroxide, and then converted to compound 64, for example, by reaction with (S)-amphetamine hemisulfate. The hydroxyl groups in compound 64 are then deprotected, for example, with trifluoroacetic acid (TFA) and water, to yield the compound of Example 7.

The compound of Example 8 can be prepared by the process shown in Scheme IIG below.

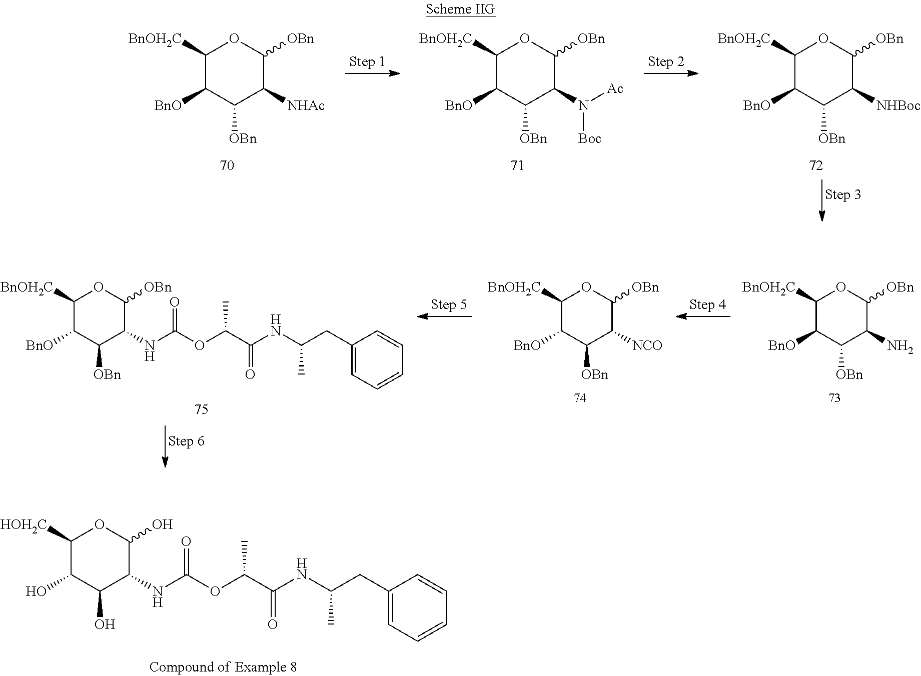

The nitrogen atom of aminosaccharide 70 is protected, for example, by reaction with di-tert-butyl dicarbonate (optionally in the presence of a catalyst such as 4-dimethylaminopyridine), to yield compound 71. The amino group is deacetylated (for example with sodium methoxide) (compound 72) and deprotected (for example with trifluoroacetic acid) (compound 73). The amino group of compound 73 is converted to an isocyanate 74, for example, by reaction with sodium bicarbonate and phosgene. Compound 74 is reacted with (2R)-2-hydroxy-N-[(2S)-1-phenylpropan-2-yl]propanamide, optionally in the presence of a base (e.g., potassium tert-butoxide), to form compound 75. The hydroxy groups of compound 75 are deprotected to yield the compound of Example 8.

The compound of Example 16 can be prepared by the process shown in Scheme IIH below.

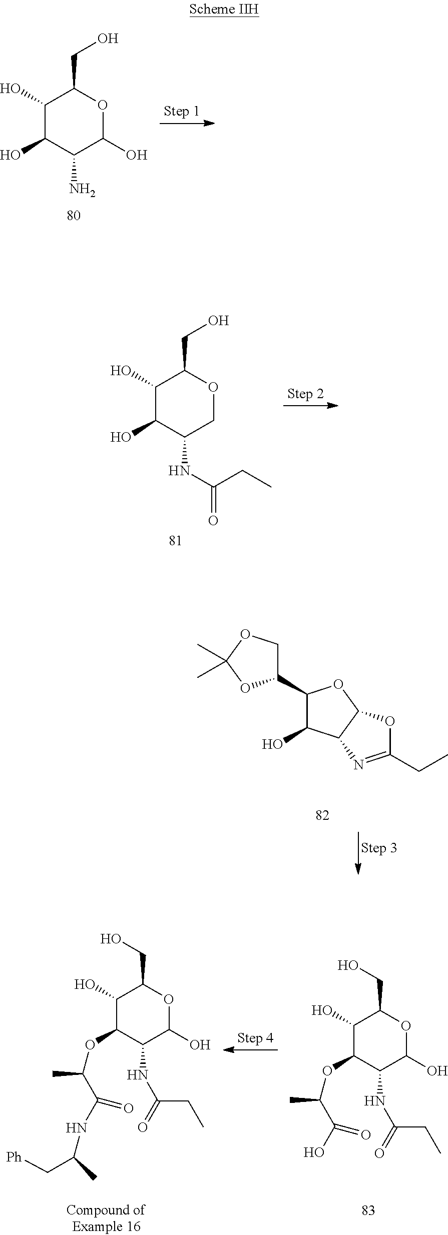 [SMILES: N[C@H]1C(O)O[C@H](CO)[C@@H](O)[C@@H]1O ; CCC(=O)N[C@H]1CO[C@H](CO)[C@@H](O)[C@@H]1O ; CCC1=N[C@H]2[C@@H](O1)O[C@H]([C@H]1COC(C)(C)O1)[C@@H]2O ; CCC(=O)N[C@H]1C(O)O[C@H](CO)[C@@H](O)[C@@H]1O[C@H](C)C(=O)N[C@@H](C)Cc1ccccc1 ; CCC(=O)N[C@H]1C(O)O[C@H](CO)[C@@H](O)[C@@H]1O[C@H](C)C(=O)O]

The amino group of aminosaccharide 80 is amidated by reaction with propionic anhydride, for example, in the presence of an alcohol (such as methanol), to yield compound 81. Compound 81 is converted to the compound 82 by reaction with dry acetone and FeCl₃. Compound 82 is converted to the compound 83, for example, by reaction with sodium hydride in dry dioxane and (S)-2-chloropropionic acid. Compound 83 is reacted with (S)-Amphetamine hemisulfate to yield the compound of Example 16.

The compounds of Examples 18 and 19 can be prepared in a manner analogous to scheme IIH.

The compound of Example 17 can be prepared by the process shown in Scheme IIJ below.

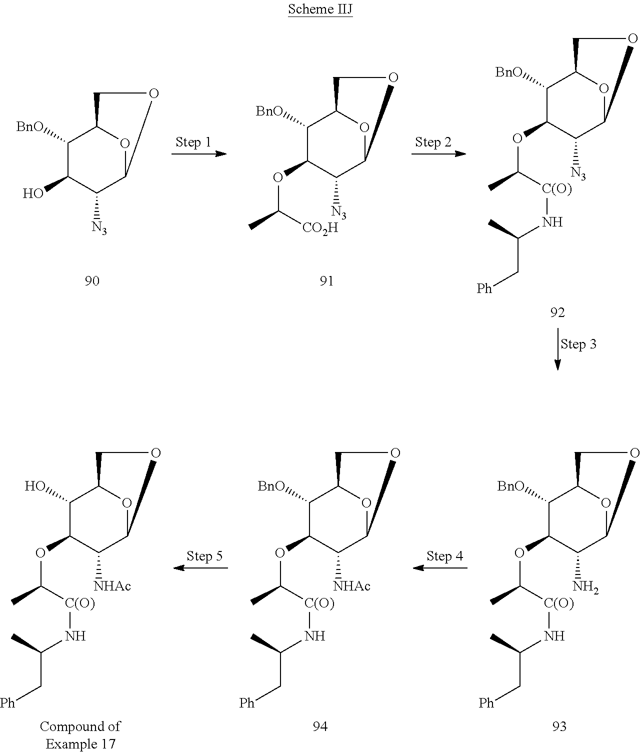

Bicyclic compound 90 is reacted with chloropropionic acid to form compound 91, which is then reacted with (S)-amphetamine hemisulfate to yield compound 92. The azide group of compound 92 is then hydrogenated to form compound 93. Compound 93 is acetylated, for example, with acetic anhydride, to yield compound 94. The benzyloxy group is deprotected, for example, by hydrogenation, to yield the compound of Example 17.

### EXAMPLES

The examples below are provided to describe specific embodiments of the present invention. By providing these specific examples, the applicants do not limit the scope and spirit of the present invention.

### Example 1

### (2R)-2-(((3R,4R,5R,6R)-3-acetamido-2,5-dihydroxy-6-(hydroxymethyl)tetrahydro-2H-pyran-4-yl)oxy)-N—((S)-1-phenylpropan-2-yl)propanamide (Compound 1)

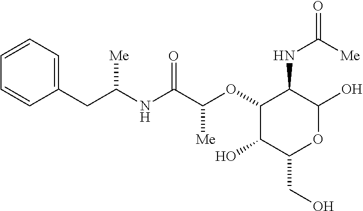

To a stirred solution of N-acetyl-muramic acid (0.36 g, 1.23 mmol) in DMF (7.5 mL) was added (benzotriazol-1-yloxy)tripyrrolidinophosphonium hexafluorophosphate (0.96 g, 1.84 mmol) followed by a solution of (S)-amphetamine (0.182 g, 1.35 mmol) and Hunig's base (0.63 g, 0.85 mL, 4.91 mmol) in DMF (3.5 mL) and the mixture was heated at 35° C. overnight. The DMF was removed in vacuo and the residual yellow oil was purified using a Biotage Isolera automated chromatography system under reversed-phase conditions (C₁₈ column, gradient of 0→100% acetonitrile in 0.1% aqueous trifluoroacetic acid] with detection at 258 nm to afford, after freeze drying, crude (2R)-2-(((3R,4R,5R,6R)-3-acetamido-2,5-dihydroxy-6-(hydroxymethyl)tetrahydro-2H-pyran-4-yl)oxy)-N—((S)-1-phenylpropan-2-yl)propanamide (0.27 g), as a yellow solid. A further batch of crude material was prepared. The combined batches of crude material were subject to further chromatographic purification to afford, after lypophilisation, (2R)-2-(((3R,4R,5R,6R)-3-acetamido-2,5-dihydroxy-6-(hydroxymethyl)tetrahydro-2H-pyran-4-yl)oxy)-N—((S)-1-phenylpropan-2-yl)propanamide (0.22 g)

¹H NMR (DMSO-d₆): δ8.22 (d, 0.75H, 0.75×NH), 8.13 (d, 0.25H, 0.25×NH), 7.43 (d, 1H), 7.25 (m, 5H), 5.05 (d, 0.75H, 0.75H), 4.40 (d, 0.25H, 0.25 CH), 4.25 (m, 0.75H, 0.75×CH), 4.04 (m, 0.25H, 0.25×CH), 3.95 (m, 1H), 3.70-3.11 (m, 6H), 2.77 (m, 1H), 2.64 (m, 1H), 1.83 (s, 3H), 1.18 (m, 3H), 1.03 (m, 3H).

MS: m/z (ES+)=433.0 [M+Na]⁺

### Example 1A

### (R)-2-(((2R,3R,4R,5R)-2-acetamido-4,5,6-trihydroxy-1-oxohexan-3-yl)oxy)-N—((R)-1-phenylpropan-2-yl)propanamide (N-acetyl muramic acid, L-amphetamine) (Compound 3)

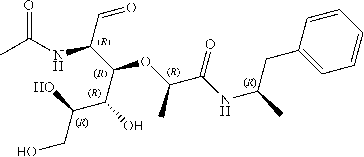

Compound 3 may be prepared by the procedure described in Example 1 using the appropriate starting materials.

¹H NMR (MeOD): δ 7.25 (m, 5H), 5.24 (d, 1H), 4.45 (q, 1H), 4.13 (m, 2H), 3.87-3.40 (m, 7H), 3.92 (dd, 1H), 3.71 (dd, 1H), 2.03 (s, 3H), 1.31 (d, 3H), 1.12 (d, 3H).

MS: m/z=432.97 [M+Na]⁺.

### Example 2

### (2R,3S,4R,5R)-2-(hydroxymethyl)-6-(octyloxy)-5-(2-(1-phenylpropan-2-yl)hydrazinyl)tetrahydro-2H-pyran-3,4-diol

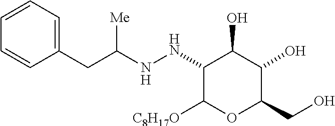

To a stirred solution of D-glucosamine hydrochloride (4.00 g, 18.5 mmol) in water (120 mL) was added benzyl chloroformate (3.16 g, 2.65 mL, 18.5 mmol) and stirring was continued overnight. The resulting precipitate was collected by suction filtration and washed with water (2×20 mL). Residual water was removed azeotropically with toluene (3×100 mL) to give N-Cbz-glucosamine (4.86 g), as a white solid that was used without further purification.

To a stirred suspension of N-Cbz-glucosamine (4.86 g, 15.5 mmol) in 1-octanol (190 mL) was added p-toluenesulfonic acid (0.47 g, 2.48 mmol) and the mixture was heated at 140° C. overnight. The 1-octanol was removed in vacuo and the resulting solid was purified by medium-pressure chromatography on silica eluting with a gradient of 5→10% methanol in dichloromethane to afford N-Cbz-C¹-octyl-glucosamine (2.39 g), as a pale-tan solid. R_{f} 0.27 [methanol-dichloromethane, 1:19 v/v].

10% Palladium on carbon (525 mg) was cautiously wetted with ethyl acetate (3 mL) under nitrogen. A solution of N-Cbz-C¹-octyl-glucoasmine (2.39 g, 5.62 mmol) in methanol (80 mL) was added, and the flask was evacuated. An atmosphere of hydrogen was introduced via a balloon and the mixture was stirred overnight at room temperature. The catalyst was removed by filtration of the suspension through a thin layer of Celite and the filtrate was concentrated to afford C¹-octyl-glucosamine (1.60 g), as an off-white solid that was used without further purification.

To a stirred solution of C¹-octyl-glucosamine (1.59 g, 5.46 mmol) and potassium hydroxide (0.12 g, 2.18 mmol) in water (4.5 ml) at 95° C. was added a solution of hydroxylamine-O-sulfonic acid (0.12 mg, 1.09 mmol) in water (1.2 mL) and stirring was continued for a further 15 min. The reaction mixture was then cooled to room temperature, neutralised with acetic acid, phenylacetone (0.15 g, 1.09 mmol) was added and stirring was continued for a further 1 h. The resulting mixture was diluted with water (10 mL) and extracted with ethyl acetate (3×25 mL). The combined organic layers were dried (MgSO₄) and concentrated. Diethyl ether (9.0 mL) was added followed by 2 M hydrogen chloride in diethyl ether (4.36 mL, 8.72 mmol) and the reaction mixture was stirred for 30 min. The resulting mixture was evaporated to dryness and residual hydrogen chloride was azeotropically removed using diethyl ether (4×10 mL) to give amphetamine-(C¹-octyl-glucosamine) hydrazone hydrochloride (390 mg), as a semi-solid that was used without further purification.

To a stirred solution of amphetamine-(C¹-octyl-glucosamine) hydrazone hydrochloride (390 mg) in THF (7 mL) was added a solution of sodium cyanoborohydride (0.12 g, 1.91 mmol) in methanol (3.5 mL) and stirring was continued for a further 1 h. The mixture was evaporated to dryness to give a pale-yellow semi-solid. To this residue was added diethyl ether (3.5 mL) followed by a solution of 2 M hydrogen chloride in diethyl ether (0.85 mL, 1.70 mmol) and the reaction mixture was stirred for 30 min. The mixture was evaporated to dryness and residual hydrogen chloride was azeotropically removed with diethyl ether (4×10 mL) to give impure amphetamine-(C¹-octyl-glucosamine) hydrazine hydrochloride (0.54 g), as a yellow semi-solid. This material was subject to purification by semi-preparative HPLC to afford after lyophisation (2R,3S,4R,5R)-2-(hydroxymethyl)-6-(octyloxy)-5-(2-(1-phenylpropan-2-yl)hydrazinyl)tetrahydro-2H-pyran-3,4-diol (0.05 g) as a semi-solid.

¹H NMR (DMSO-d₆+D₂O): 7.24 (m, 5H), 4.86 (m, 1H), 3.64-3.31 (m, 7H), 3.16 (m, 2H), 2.92 (m, 1H) 2.62 (m, 1H), 1.53 (m, 2H), 1.23 (m, 10H), 1.10 (m, 3H) 0.83 (m, 3H).

MS: m/z (ES+)=425.05 [M+H]⁺

### Example 3

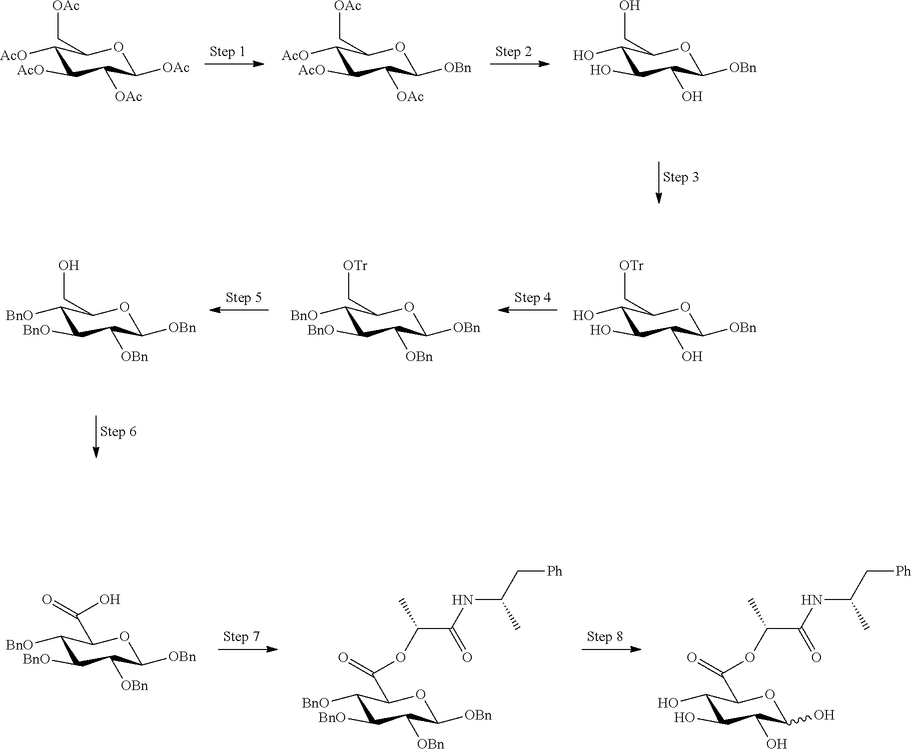

### Step 1

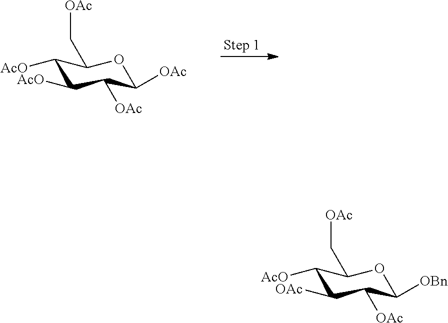

### (2R,3R,4S,5R,6R)-2-(Acetoxymethyl)-6-(benzyloxy)tetrahydro-2H-pyran-3,4,5-triyl triacetate

(2R,3R,4S,5R,6R)-6-(Acetoxymethyl)tetrahydro-2H-pyran-2,3,4,5-tetrayl tetraacetate (16.0 g, 40.99 mmol) and benzyl alcohol (4.26 mL, 4.43 g, 40.99 mmol) were dissolved in DCM (40 mL) and boron trifluoride diethyletherate (5.09 mL, 5.82 g, 40.99 mmol) was added. The solution was stirred overnight at room temperature. After 24 hours the reaction mixture was quenched with NaHCO₃ (saturated aqueous, 20 mL). The DCM layer was separated and washed with NaHCO₃ (saturated aqueous, 20 mL) and water (20 mL). The DCM layer was dried (MgSO₄) and concentrated. The product was purified by flash column chromatography (SiO₂, eluting with ethyl acetate/petroleum ether 25-40% v/v) to give product which was crystallised from ethanol, filtered and washed with petroleum ether and dried for 1 hour at 40° C. This gave (2S,3R,4S,5R,6R)-6-((benzyloxy)methyl)tetrahydro-2H-pyran-2,3,4,5-tetrayl tetraacetate (9.25 g).

¹H NMR (CDCl₃): δ 7.31 (m, 5H), 5.15 (m, 3H), 4.92 (d, 1H), 4.62 (d, 1H), 4.54 (d, 1H), 4.22 (m, AB, 2H), 3.67 (m, 1H), 2.13 (s, 3H), 2.07-1.98 (3s, 9H).

### Step 2

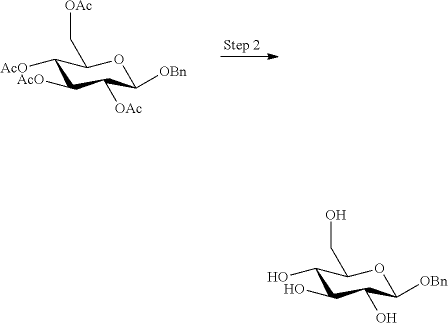

### (2R,3R,4S,5S,6R)-2-(Benzyloxy)-6-(hydroxymethyl)tetrahydro-2H-pyran-3,4,5-triol

(2S,3R,4S,5R,6R)-6-((benzyloxy)methyl)tetrahydro-2H-pyran-2,3,4,5-tetrayl tetra-acetate (4.68 g, 10.67 mmol) was dissolved in methanol and sodium methoxide (50 mg, 1.07 mmol) was added. After stirring overnight the reaction was neutralised using DOWEX sulfonic acid resin. Concentration of the resulting solution gave (2R,3R,4S,5S,6R)-2-(benzyloxy)-6-(hydroxymethyl)tetrahydro-2H-pyran-3,4,5-triol (2.17 g).

¹H NMR (MeOD): δ 7.35 (m, 5H), 4.95 (d, 1H), 4.66 (d, 1H), 4.35 (d, 1H), 3.90 (d, 1H), 3.30 (m, 5H).

### Step 3

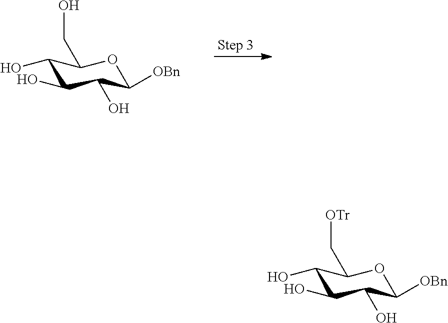

### (2R,3R,4S,5S,6R)-2-(benzyloxy)-6-((trityloxy)methyl)tetrahydro-2H-pyran-3,4,5-triol

(2R,3R,4S,5S,6R)-2-(benzyloxy)-6-(hydroxymethyl)tetrahydro-2H-pyran-3,4,5-triol (2.17 g, 8.03 mmol) was dissolved in DMF (30 mL) and triethylamine (1.12 mL, 0.812 g, 8.02 mmol) and trityl chloride (2.24 g, 8.03 mmol) were added The reaction mixture was stirred at 20° C. overnight. The solvent was removed under high vacuum and the residue partitioned between water (50 mL) and ethyl acetate (200 mL). The ethyl acetate layer was dried (MgSO₄) and concentrated in vacuo. The product was purified by flash column chromatography (SiO₂, eluting with chloroform/methanol 9:1 v:v) to give (2R,3R,4S,5S,6R)-2-(benzyloxy)-6-((trityloxy)methyl)-tetrahydro-2H-pyran-3,4,5-triol (3.38 g).

¹H NMR (CDCl₃): δ 7.41-7.18 (m, 20H), 4.95 (d, 1H), 4.67 (d, 1H), 4.39 (d, 1H), 3.70-3.36 (m, 6H), 2.80 (d, 1H), 2.72 (d, 1H), 2.48 (d, 1H).

### Step 4

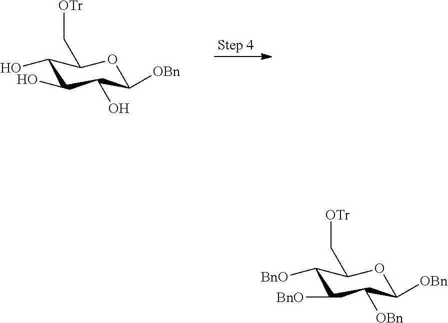

### (2R,3R,4S,5R,6R)-2,3,4,5-Tetrakis(benzyloxy)-6-((trityloxy)methyl)tetrahydro-2H-pyran

(2R,3R,4S,5S,6R)-2-(Benzyloxy)-6-((trityloxy)methyl)tetrahydro-2H-pyran-3,4,5-triol (3.18 g, 6.20 mmol) was dissolved in DMF (60 mL) and sodium hydride (0.99 g, 60%, 24.82 mmol) was added taking care because of the vigorous effervescence. Benzyl bromide (2.21 mL, 3.18 g, 18.61 mmol) was added and the reaction mixture was stirred overnight. Water was added at 0° C., again effervescence occurred and the product was extracted with TBME (2×50 mL). The TBME layer was washed with brine (20 mL), dried (MgSO₄) and concentrated in vacuo. The product was purified by flash column chromatography (SiO₂, eluting with petroleum ether/ethyl acetate 9:1 v:v) to give (2R,3R,4S,5R,6R)-2,3,4,5-tetrakis(benzyloxy)-6-((trityloxy)methyl)-tetrahydro-2H-pyran (4.86 g).

¹H NMR (CDCl₃): δ 7.60-7.14 (m, 33H), 6.88 (dd, 2H), 5.05 (m, 2H), 4.97 (d, 1H), 4.82-4.66 (m, 4H), 4.58 (d, 1H), 4.37 (d, 1H), 3.83 (m, 1H), 3.62 (m, 3H), 3.43 (m, 1H), 3.29 (dd, 1H).

### Step 5

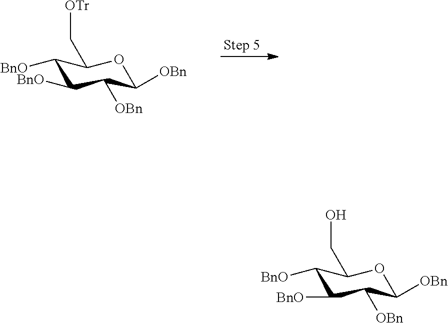

### ((2R,3R,4S,5R,6R)-3,4,5,6-Tetrakis(benzyloxy)tetrahydro-2H-pyran-2-yl)methanol

(2R,3R,4S,5R,6R)-2,3,4,5-tetrakis(benzyloxy)-6-((trityloxy)methyl)tetrahydro-2H-pyran (3.27 g, 4.18 mmol) was dissolved in acetic acid (20 mL), water (4 mL) and DCM (10 mL). Trifluoroacetic acid was added until a strong, persistent yellow colour was observed. The solution was stirred for 2 hours and after this time, water (20 mL) was added and the solution concentrated to dryness. Co-evaporation from water (20 mL) and toluene (50 mL) gave the product. The product was purified by flash column chromatography (SiO₂, eluting with petroleum DCM/methanol 99:1 v/v) to give ((2R,3R,4S,5R,6R)-3,4,5,6-tetrakis(benzyloxy)tetrahydro-2H-pyran-2-yl)methanol (1.50 g).

¹H NMR (CDCl₃): δ 7.65-7.00 (m, 20H), 5.05-4.50 (m, 8H), 3.90 (d, 1H), 3.80-3.42 (m, 5H), 3.36 (m, 1H).

### Step 6

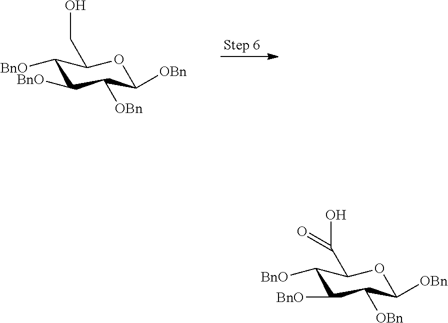

### (2S,3S,4S,5R,6R)-3,4,5,6-Tetrakis(benzyloxy)tetrahydro-2H-pyran-2-carboxylic acid

((2R,3R,4S,5R,6R)-3,4,5,6-Tetrakis(benzyloxy)tetrahydro-2H-pyran-2-yl)methanol (0.67 g, 1.24 mmol) was dissolved in acetone (30 mL) and a solution of potassium dichromate (0.62 g, 1.24 mmol) in 6M sulphuric acid (9 mL) was added. The reaction mixture was heated at 55° C. for 2 hours. The reaction was diluted with water (300 mL) and extracted with DCM (3×100 mL). The combined organic layers were dried (MgSO₄) and concentrated. The product was purified by flash column chromatography (SiO₂, eluting with acetone/petroleum ether 3:7 v/v) to give (2S,3S,4S,5R,6R)-3,4,5,6-tetrakis(benzyloxy)tetrahydro-2H-pyran-2-carboxylic acid (0.44 g).

¹H NMR (CDCl₃): δ 7.45-7.10 (m, 20H), 4.95-4.43 (m, 9H), 3.92 (d, 1H), 3.76 (dd, 1H), 3.62 (dd, 1H), 3.46 (dd, 1H).

### Step 7

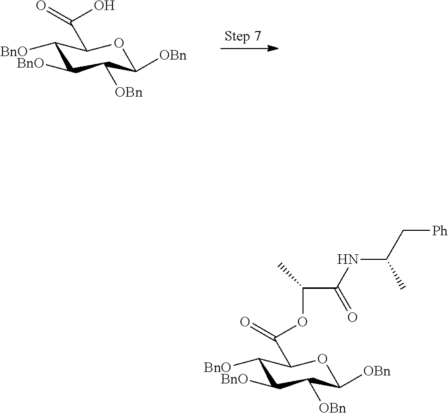

### (2S,3S,4S,5R,6R)—(R)-1-oxo-1-(((S)-1-phenylpropan-2-yl)amino)propan-2-yl-3,4,5,6-tetrakis(benzyloxy)tetrahydro-2H-pyran-2-carboxylate

(2S,3S,4S,5R,6R)-3,4,5,6-tetrakis(benzyloxy)tetrahydro-2H-pyran-2-carboxylic acid (0.44 g, 0.79 mmol) and (R)-2-hydroxy-N—((S)-1-phenylpropan-2-yl)propanamide (0.16 g, 0.79 mmol) were dissolved in acetonitrile (9 mL) and EDC (0.17 g, 0.87 mmol) was added. DMAP (0.01 g, 0.079 mmol) was added and the reaction mixture was stirred overnight. The reaction mixture was partitioned between EtOAc (50 mL) and aqueous saturated sodium bicarbonate (20 mL). The ethyl acetate layer was washed with brine (20 mL), dried (MgSO₄) and concentrated in vacuo to give product. The product was purified by flash column chromatography (SiO₂, eluting with acetone/petroleum ether 1:3 v/v) to give (2S,3S,4S,5R,6R)—(R)-1-oxo-1-(((S)-1-phenylpropan-2-yl)amino)propan-2-yl 3,4,5,6-tetrakis(benzyloxy)tetrahydro-2H-pyran-2-carboxylate (0.41 g).

¹H NMR (CDCl₃): δ 7.36-7.00 (m, 25H), 5.97 (d, 1H), 5.16 (q, 1H), 4.90-4.47 (m, 9H), 4.17 (m, 1H), 3.83 (m, 2H), 3.62 (dd, 1H), 3.38 (dd, 1H), 2.73-2.50 (m, 2H), 1.33 (d, 3H), 0.97 (d, 3H).

### Step 8

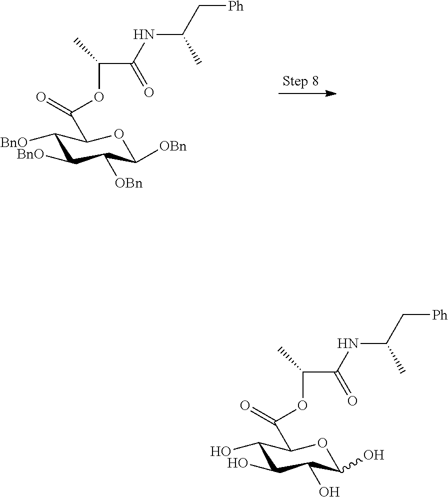

### (2S,3S,4S,5R)—(R)-1-oxo-1-(((S)-1-phenylpropan-2-yl)amino)propan-2-yl 3,4,5,6-tetrahydroxytetrahydro-2H-pyran-2-carboxylate

(2S,3S,4S,5R,6R)—(R)-1-oxo-1-(((S)-1-phenylpropan-2-yl)amino)propan-2-yl 3,4,5,6-tetrakis(benzyloxy)tetrahydro-2H-pyran-2-carboxylate (0.20 g, 0.27 mmol) was dissolved in THF (8 mL), water (8 mL) and formic acid (1 mL). Palladium hydroxide (20% w/w on carbon, 0.94 g) was added and the mixture was hydrogenated for 2 hours. Water (5 mL) was added and hydrogenation then continued overnight. The catalyst was removed by filtration and the solvent evaporated to give product which was purified by preparative HPLC (Phenomenex Kinetex C18 column using a gradient of 10-100% acetonitrile/0.1% TFA in water as eluent) to give (2S,3S,4S,5R)—(R)-1-oxo-1-(((S)-1-phenylpropan-2-yl)amino)propan-2-yl 3,4,5,6-tetrahydroxytetrahydro-2H-pyran-2-carboxylate (0.103 g).

¹H NMR (D₂O): δ 7.30-7.10 (m, 5H), 5.18 (d, 1H), 4.95 (m, 1H), 4.60 (d, 1H), 4.33 (d, 1H), 4.15-3.90 (m, 2H), 3.65 (m, 1H), 3.52-3.40 (m, 2H), 3.20 (m, 1H), 2.86-2.50 (m, 2H), 1.24 (d, 3H), 1.08 (m, 3H).

MS: m/z=405.89 [M+Na]⁺.

### Example 4

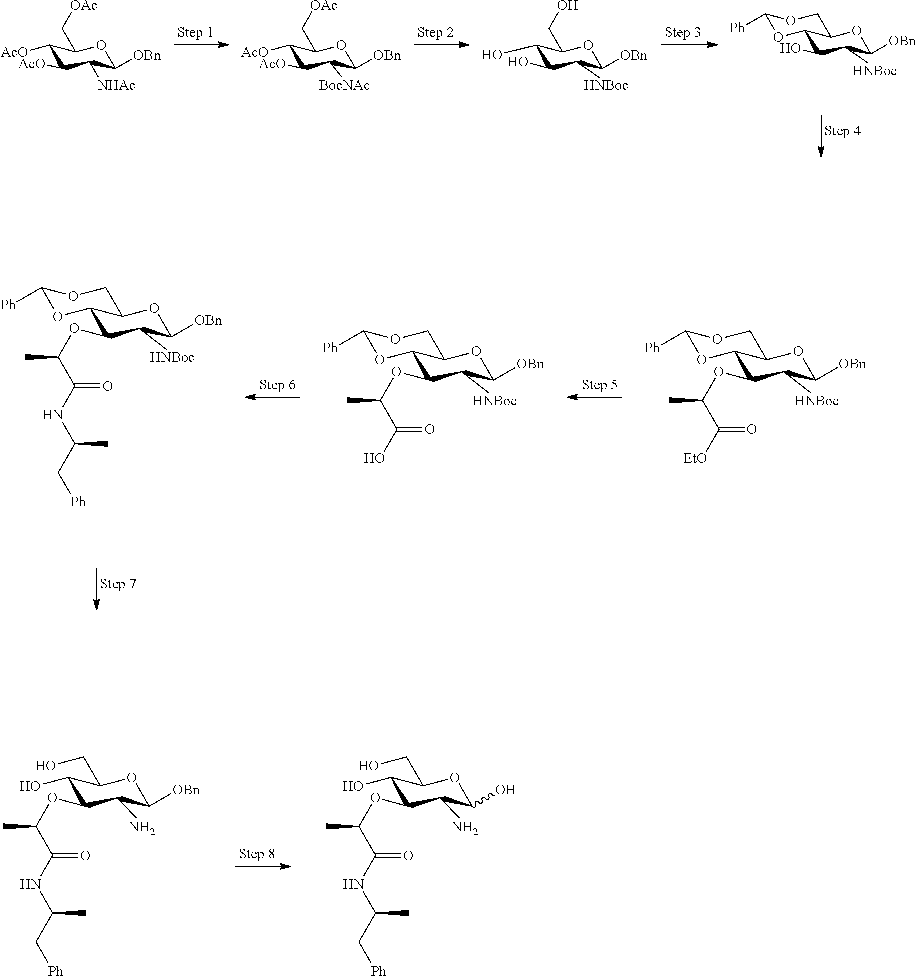

### Step 1

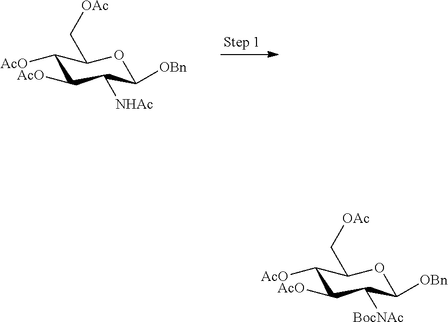

### (2R,3S,4R,5R,6R)-2-(acetoxymethyl)-6-(benzyloxy)-5-(N-(tert-butoxycarbonyl)acetamido)tetrahydro-2H-pyran-3,4-diyl diacetate

(2R,3S,4R,5R,6R)-5-acetamido-2-(acetoxymethyl)-6-(benzyloxy)tetrahydro-2H-pyran-3,4-diyl diacetate (10.0 g, 22.86 mmol) was dissolved in THF (100 mL) and di-tert-butyl dicarbonate (24.95 g, 114.30 mmol) was added. 4-Dimethylaminopyridine (0.28 g, 2.29 mmol) was added and the solution was heated at 60° C. for 24 hours. The solution was concentrated to remove the di-tert-butyl dicarbonate and the product was purified by flash column chromatography (SiO₂, eluting with acetone:petroleum ether 2:7 v/v) to give (2R,3S,4R,5R,6R)-2-(acetoxymethyl)-6-(benzyloxy)-5-(N-(tert-butoxycarbonyl)acetamido)tetrahydro-2H-pyran-3,4-diyl diacetate (13.43 g).

¹H NMR (CDCl₃): δ 7.30 (m, 5H), 5.71 (m, 1H), 5.42 (d, 1H), 5.11 (m, 1H), 5.06 (m, 1H), 4.87 (m, 1H), 4.59 (m, 1H), 4.40-4.10 (m, 2H), 3.74 (m, 1H), 2.45-1.95 (m, 12H), 1.58-1.38 (m, 9H).

### Step 2

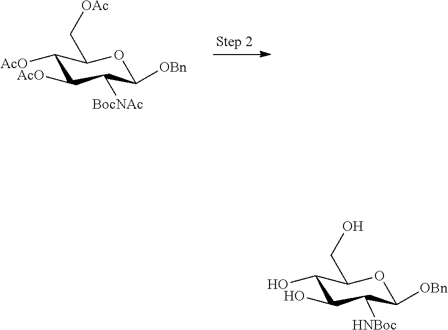

### tert-butyl ((2R,3R,4R,5S,6R)-2-(benzyloxy)-4,5-dihydroxy-6-(hydroxymethyl)tetrahydro-2H-pyran-3-yl)carbamate

(2R,3S,4R,5R,6R)-2-(acetoxymethyl)-6-(benzyloxy)-5-(N-(tert-butoxycarbonyl)acetamido)tetrahydro-2H-pyran-3,4-diyl diacetate (13.43 g, 24.98 mmol) was dissolved in methanol (75 mL) and sodium methoxide (0.14 g, 2.50 mmol) was added. After stirring for 3 hours the reaction was neutralised using DOWEX sulfonic acid resin. Evaporation of the solvents gave tert-butyl ((2R,3R,4R,5S,6R)-2-(benzyloxy)-4,5-dihydroxy-6-(hydroxymethyl)tetrahydro-2H-pyran-3-yl)carbamate (8.39 g).

¹H NMR (MeOD): δ 7.30 (m, 5H), 4.95 (d, 1H), 4.72 (d, 1H), 4.41 (d, 1H), 3.92 (d, 1H), 3.69 (dd, 1H), 3.30 (m, 4H), 1.45 (s, 9H).

### Step 3

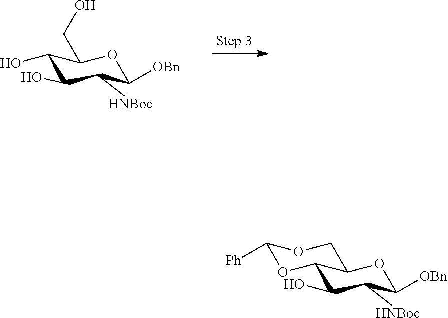

### tert-butyl ((2R,4aR,6R,7R,8R,8aS)-6-(benzyloxy)-8-hydroxy-2-phenylhexahydropyrano[3,2-d][1,3]dioxin-7-yl)carbamate

tert-butyl ((2R,3R,4R,5S,6R)-2-(benzyloxy)-4,5-dihydroxy-6-(hydroxymethyl)tetrahydro-2H-pyran-3-yl)carbamate (8.39 g, 22.71 mmol) was suspended in acetonitrile (60 mL) and benzaldehyde dimethyl acetal (3.41 mL, 3.46 g, 22.71 mmol) was added followed by para toluenesulfonic acid (0.39 g, 2.27 mmol). Stirring was continued overnight. The reaction was concentrated to dryness and the product was purified by flash column chromatography (SiO₂, eluting with MeOH/DCM ether 5:95 v/v), re-crystallised from ethanol and dried at 40° C. to give tert-butyl ((2R,4aR,6R,7R,8R,8aS)-6-(benzyloxy)-8-hydroxy-2-phenylhexahydropyrano[3,2-d][1,3]dioxin-7-yl)carbamate (7.20 g).

¹H NMR (CDCl₃): δ 7.41 (d, 2H), 7.30 (m, 8H), 5.59 (s, 1H), 4.94 (d, 1H), 4.70 (m, 2H), 4.62 (d, 1H), 4.38 (dd, 1H), 4.05 (m, 1H), 3.83 (dd, 1H), 3.59 (dd, 1H), 3.43-3.33 (m, 3H), 1.44 (s, 9H).

### Step 4

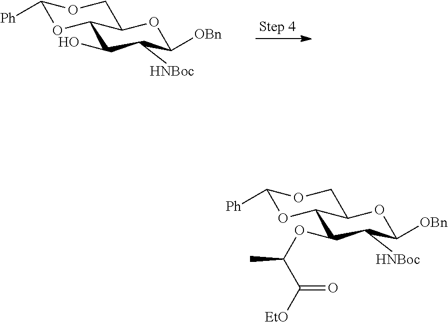

### (R)-ethyl 2-(((2R,4aR,6R,7R,8R,8aS)-6-(benzyloxy)-7-((tert-butoxycarbonyl)amino)-2-phenylhexahydropyrano[3,2-d][1,3]dioxin-8-yl)oxy)propanoate

tert-butyl ((2R,4aR,6R,7R,8R,8aS)-6-(benzyloxy)-8-hydroxy-2-phenylhexahydropyrano[3,2-d][1,3]dioxin-7-yl)carbamate (3.29 g, 7.19 mmol) was dissolved in THF (100 mL). Sodium hydride (0.86 g, 60% w/v, 21.57 mmol) was added at −20° C. After 30 minutes (S)-ethyl 2-(((trifluoromethyl)sulfonyl)oxy)propanoate (5.40 g, 21.57 mmol) was added over 30 minutes at −20° C. as a solution in THF (10 mL). At the end of the addition the solvent was evaporated, the reaction mixture was partitioned between ethyl acetate (200 mL) and brine (50 mL). The ethyl acetate layer was washed with brine (50 mL), dried (MgSO₄) and concentrated. The product was purified by flash column chromatography (SiO₂, eluting with 10-40% ethyl acetate in petroleum ether) to give (R)-ethyl 2-(((2R,4aR,6R,7R,8R,8aS)-6-(benzyloxy)-7-((tert-butoxycarbonyl)amino)-2-phenylhexahydropyrano[3,2-d][1,3]dioxin-8-yl)oxy)propanoate (2.71 g). NMR and LCMS suggested mainly one product but also the presence of a minor diasteromer.

¹H NMR (CDCl₃): δ 7.40 (m, 10H), 5.59 (s, 1H), 5.39 (d, 1H), 4.94 (d, 1H), 4.71 (m, 1H), 4.46 (q, 1H), 4.38 (dd, 1H), 4.15 (m, 2H), 4.00-3.75 (m, 2H), 3.66 (dd, 1H), 3.50-3.37 (m, 1H), 1.50-1.40 (m, 9H), 1.42 (d, 3H), 1.27 (t, 3H).

MS: m/z=580.2 [M+Na]⁺.

### Step 5

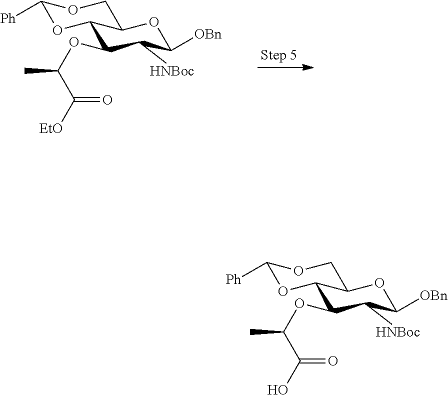

### (R)-2-(((2R,4aR,6R,7R,8R,8aS)-6-(benzyloxy)-7-((tert-butoxycarbonyl)amino)-2-phenylhexahydropyrano[3,2-d][1,3]dioxin-8-yl)oxy)propanoic acid

(R)-ethyl 2-(((2R,4aR,6R,7R,8R,8aS)-6-(benzyloxy)-7-((tert-butoxycarbonyl)amino)-2-phenylhexahydropyrano[3,2-d][1,3]dioxin-8-yl)oxy)propanoate (2.71 g, 4.86 mmol) was dissolved in methanol (15 mL), water (15 mL) and THF (20 mL). Potassium hydroxide (1.09 g, 19.44 mmol) was added and the mixture was stirred overnight at room temperature. The solvent was evaporated and the reaction partitioned between ethyl acetate (100 mL) and 1M hydrochloric acid (50 mL). The ethyl acetate layer was washed with brine (30 mL), dried (MgSO₄) and concentrated to give ((R)-2-(((2R,4aR,6R,7R,8R,8aS)-6-(benzyloxy)-7-((tert-butoxycarbonyl)amino)-2-phenylhexahydropyrano[3,2-d][1,3]dioxin-8-yl)oxy)propanoic acid (2.57 g).

¹H NMR (CDCl₃): δ 7.30 (m, 10H), 5.59 (s, 1H), 4.84 (d, 1H), 4.80-4.50 (m, 1H), 4.52 (d, 1H), 4.32 (dd, 1H), 4.25 (q, 1H), 3.85 (dd, 1H), 4.00-3.75 (m, 2H), 3.66 (dd, 1H), 3.56-3.37 (m, 2H), 1.39 (m, 9H), 1.42 (d, 3H).

### Step 6

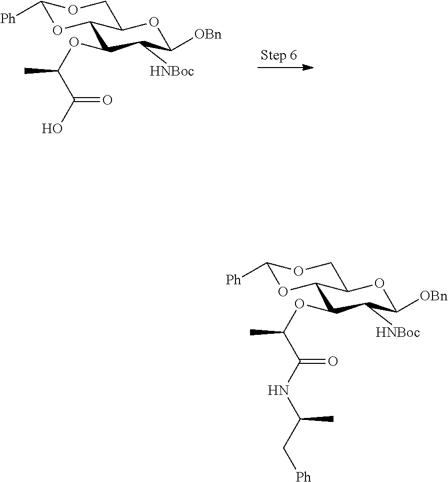

### tert-butyl ((2R,4aR,6R,7R,8R,8aS)-6-(benzyloxy)-8-(((R)-1-oxo-1-(((S)-1-phenylpropan-2-yl)amino)propan-2-yl)oxy)-2-phenylhexahydropyrano[3,2-d][1,3]dioxin-7-yl)carbamate

((R)-2-(((2R,4aR,6R,7R,8R,8aS)-6-(benzyloxy)-7-((tert-butoxycarbonyl)amino)-2-phenylhexahydropyrano[3,2-d][1,3]dioxin-8-yl)oxy)propanoic acid (2.57 g, 4.86 mmol) and (S)-amphetamine (0.99 g, 7.29 mmol) were dissolved in DCM (30 mL) and N-(3-Dimethylaminopropyl)-N′-ethylcarbodiimide hydrochloride (0.93 g, 7.29 mmol) and 4-(Dimethylamino)pyridine (0.12 g, 0.97 mmol) were added. After 3 hours aqueous NaHCO₃ (50 mL), DCM (50 mL) and THF (40 mL) were added. The organic layer was separated and washed with brine (30 mL), dried (MgSO₄) and concentrated. The resulting white solid was slurried in hot methanol, collected by filtration and washed with cold methanol to give tert-butyl ((2R,4aR,6R,7R,8R,8aS)-6-(benzyloxy)-8-(((R)-1-oxo-1-(((S)-1-phenylpropan-2-yl)amino)propan-2-yl)oxy)-2-phenylhexahydropyrano[3,2-d][1,3]dioxin-7-yl)carbamate (1.23 g).

¹H NMR (DMSO): δ 7.45-7.10 (m, 15H), 7.06 (d, 1H), 6.98 (d, 1H), 5.71 (s, 1H), 4.81 (d, 1H), 4.61 (dd, 1H), 4.52 (d, 1H), 4.29 (dd, 1H), 4.05 (q, 1H), 3.86 (m, 1H), 3.82 (dd, 1H), 3.67 (dd, 1H), 3.25-3.65 (m, 3H), 2.65 (m, 2H), 1.39 (s, 9H), 1.10 (2d, 6H).

MS: m/z=669.3 [M+Na]⁺.

### Step 7

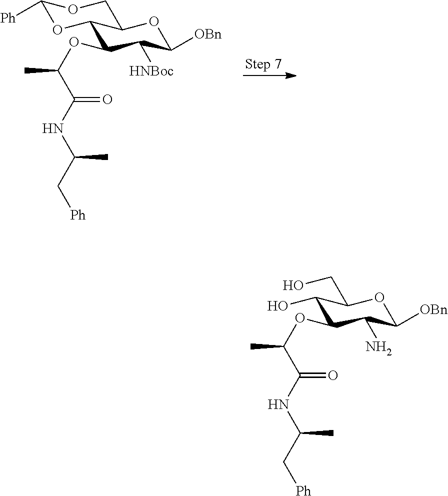

### (R)-2-(((2R,3R,4R,5S,6R)-3-amino-2-(benzyloxy)-5-hydroxy-6-(hydroxymethyl)tetrahydro-2H-pyran-4-yl)oxy)-N—((S)-1-phenylpropan-2-yl)propanamide

tert-butyl ((2R,4aR,6R,7R,8R,8aS)-6-(benzyloxy)-8-(((R)-1-oxo-1-(((S)-1-phenylpropan-2-yl)amino)propan-2-yl)oxy)-2-phenylhexahydropyrano[3,2-d][1,3]dioxin-7-yl)carbamate (0.90 g, 1.39 mmol) was dissolved in methanol and para toluenesulfonic acid (0.27 g, 1.39 mmol) was added. The solution was heated at 70° C. for 1.5 hours. Triethylamine was added to neutralise the reaction mixture and the solvent was evaporated. The product was purified by flash column chromatography (SiO₂, eluting with methanol/ethyl acetate 5:95 v/v) to give (R)-2-(((2R,3R,4R,5S,6R)-3-amino-2-(benzyloxy)-5-hydroxy-6-(hydroxymethyl)tetrahydro-2H-pyran-4-yl)oxy)-N—((S)-1-phenylpropan-2-yl)propanamide (0.433 g).

¹H NMR (MeOD): δ 7.45-7.00 (m, 5H), 5.05 (d, 1H), 4.95 (m, 2H), 4.62 (d, 1H), 4.36 (q, 1H), 4.21 (d, 1H), 4.05 (q, 1H), 3.82 (d, 1H), 3.71 (dd, 1H), 3.41-3.30 (m, 3H), 3.23 (m, 1H), 3.02-2.80 (m, 3H), 2.73 (dd, 1H), 2.49 (dd, 1H), 1.32 (d, 3H), 1.19 (d, 3H).

### Step 8

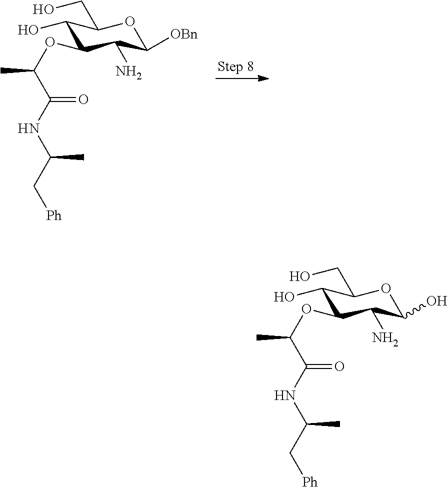

### (2R)-2-(((3R,4R,5S,6R)-3-amino-2,5-dihydroxy-6-(hydroxymethyl)tetrahydro-2H-pyran-4-yl)oxy)-N—((S)-1-phenylpropan-2-yl)propanamide

(R)-2-(((2R,3R,4R,5S,6R)-3-amino-2-(benzyloxy)-5-hydroxy-6-(hydroxymethyl)tetrahydro-2H-pyran-4-yl)oxy)-N—((S)-1-phenylpropan-2-yl)propanamide (0.300 g, 0.654 mmol) was dissolved in tetrahydrofuran (10 mL) and water (10 mL) and the palladium hydroxide (0.230 g, 0.237 mmol) was added. The reaction mixture was hydrogenated for 4 hours and the catalyst was removed by filtration on celite and the product was concentrated to dryness. The product was purified by preparative HPLC (Phenomenex Kinetex C18 column using a gradient of 10-50% acetonitrile/0.1% 10 mM NH₄HCO₃ in water as eluent). Evaporating the fractions, the product contained 7-8% acetamide. The solid product was triturated with acetonitrile (2×20 mL) to give (2R)-2-(((3R,4R,5S,6R)-3-amino-2,5-dihydroxy-6-(hydroxymethyl)tetrahydro-2H-pyran-4-yl)oxy)-N—((S)-1-phenylpropan-2-yl)propanamide (50 mg).

¹H NMR (D₂O): δ 7.20 (m, 5H), 5.02 (d α-anomer, 1H), 4.30 (d β-anomer, 1H), 4.17 (q, 1H), 4.05 (m, 1H), 3.65 (m, 3H), 3.30 (m, 1H), 3.06 (dd, 1H), 2.82 (m, 1H), 2.71-2.28 (m, 2H), 1.11 (2d, 6H).

MS: m/z=368.91 [M+H]⁺.

### Example 5

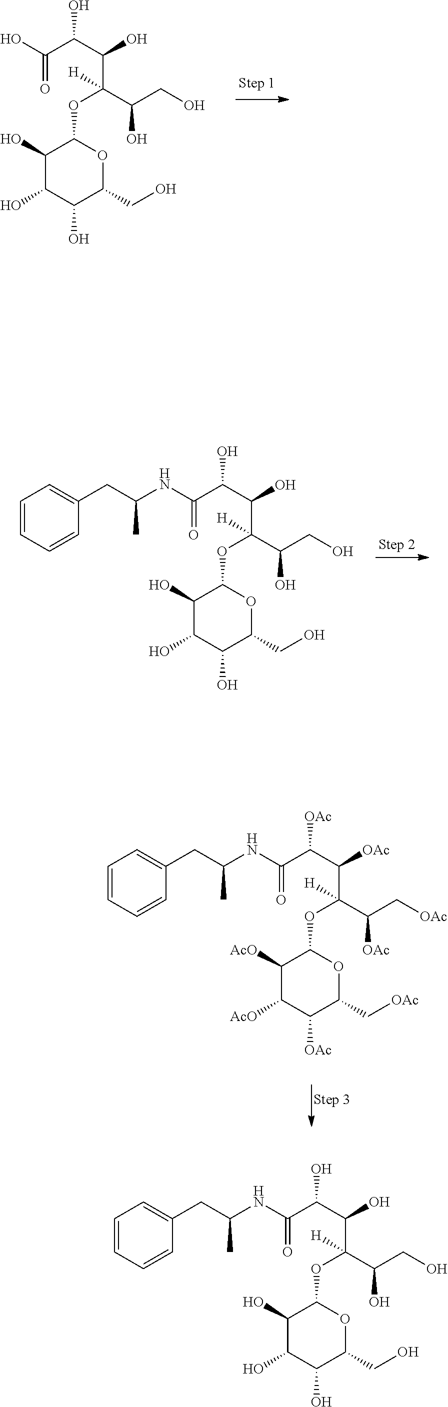 [SMILES: O=C(O)[C@H](O)[C@@H](O)[C@H](O[C@@H]1O[C@H](CO)[C@H](O)[C@H](O)[C@H]1O)[C@H](O)CO ; C[C@@H](Cc1ccccc1)NC(=O)[C@H](O)[C@@H](O)[C@H](O[C@@H]1O[C@H](CO)[C@H](O)[C@H](O)[C@H]1O)[C@H](O)CO ; CC(=O)OC[C@H]1O[C@@H](O[C@@H]([C@H](OC(C)=O)[C@@H](OC(C)=O)C(=O)N[C@@H](C)Cc2ccccc2)[C@@H](COC(C)=O)OC(C)=O)[C@H](OC(C)=O)[C@@H](OC(C)=O)[C@H]1OC(C)=O ; C[C@@H](Cc1ccccc1)NC(=O)[C@H](O)[C@@H](O)[C@H](O[C@@H]1O[C@H](CO)[C@H](O)[C@H](O)[C@H]1O)[C@H](O)CO]

### Step 1

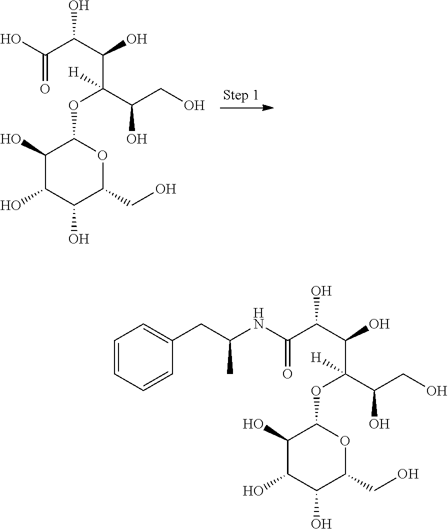 [SMILES: O=C(O)[C@H](O)[C@@H](O)[C@H](O[C@@H]1O[C@H](CO)[C@H](O)[C@H](O)[C@H]1O)[C@H](O)CO ; C[C@@H](Cc1ccccc1)NC(=O)[C@H](O)[C@@H](O)[C@H](O[C@@H]1O[C@H](CO)[C@H](O)[C@H](O)[C@H]1O)[C@H](O)CO]

### (2R,3R,4R,5R)-2,3,5,6-tetrahydroxy-N—((S)-1-phenylpropan-2-yl)-4-(((2S,3R,4S,5R,6R)-3,4,5-trihydroxy-6-(hydroxymethyl)tetrahydro-2H-pyran-2-yl)oxy)hexanamide

Lactobionic acid (2.00 g, 5.58 mmol), (S)-amphetamine (0.06 g, 5.58 mmol), HATU (2.12 g, 5.58 mmol) and N-methylmorpholine (1.13 g, 11.16 mmol) were combined in DMF (100 mL) After 4 days at 50° C. the reaction mixture was concentrated to dryness, then dissolved in methanol and passed through 3 separate SCX cartridges (5 g). Evaporation gave a solid. Crystallisation was attempted from ethanol/H₂O but very little product precipitated. The reaction mixture was re-concentrated to dryness under high vacuum to give crude (2R,3R,4R,5R)-2,3,5,6-tetrahydroxy-N—((S)-1-phenylpropan-2-yl)-4-(((2S,3R,4S,5R,6R)-3,4,5-trihydroxy-6-(hydroxymethyl)tetrahydro-2H-pyran-2-yl)oxy)hexanamide.

### Step 2

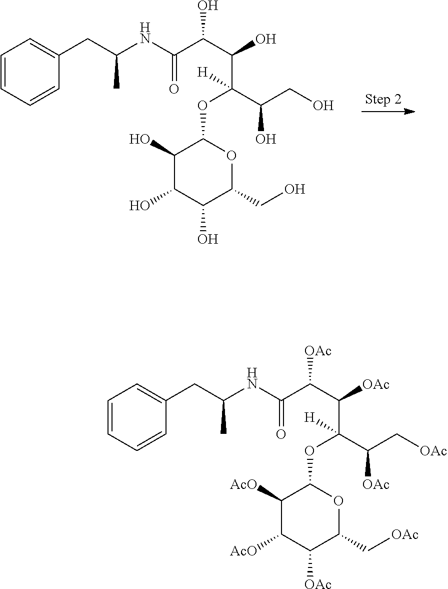 [SMILES: C[C@@H](Cc1ccccc1)NC(=O)[C@H](O)[C@@H](O)[C@H](O[C@@H]1O[C@H](CO)[C@H](O)[C@H](O)[C@H]1O)[C@H](O)CO ; CC(=O)OC[C@H]1O[C@@H](O[C@@H]([C@H](OC(C)=O)[C@@H](OC(C)=O)C(=O)N[C@@H](C)Cc2ccccc2)[C@@H](COC(C)=O)OC(C)=O)[C@H](OC(C)=O)[C@@H](OC(C)=O)[C@H]1OC(C)=O]

### (2R,3R,4S,5R)-6-oxo-6-(((S)-1-phenylpropan-2-yl)amino)-3-(((2S,3R,4S,5S,6R)-3,4,5-triacetoxy-6-(acetoxymethyl)tetrahydro-2H-pyran-2-yl)oxy)hexane-1,2,4,5-tetrayl tetraacetate

Crude (2R,3R,4R,5R)-2,3,5,6-tetrahydroxy-N—((S)-1-phenylpropan-2-yl)-4-(((2S,3R,4S,5R,6R)-3,4,5-trihydroxy-6-(hydroxymethyl)tetrahydro-2H-pyran-2-yl)oxy)hexanamide was dissolved in pyridine (100 mL) and treated with acetic anhydride (11.4 g, 0.112 mmol) overnight at 50° C. The solvent was removed under vacuum and the product was dissolved in ethyl acetate (200 mL). The ethyl acetate layer was washed with aqueous sodium hydrogencarbonate (20 mL) and brine (20 mL), dried (MgSO₄) and concentrated. The product was purified by preparative HPLC (Phenomenex Kinetex C18 column, using a 10-100% gradient of 0.1% TFA/acetonitrile in 0.1% TFA/water as eluent). Evaporating the required fractions gave (2R,3R,4S,5R)-6-oxo-6-(((S)-1-phenylpropan-2-yl)amino)-3-(((2S,3R,4S,5S,6R)-3,4,5-triacetoxy-6-(acetoxymethyl)tetrahydro-2H-pyran-2-yl)oxy)hexane-1,2,4,5-tetrayl tetraacetate (254 mg).

¹H NMR (CDCl₃): δ 7.15 (m, 5H), 6.14 (d, 1H), 5.48 (m, 2H), 5.31 (m, 1H), 5.09 (dd, 1H), 4.92 (m, 2H), 4.55 (d, 1H), 4.47 (dd, 1H), 4.23 (m, 1H), 4.20-3.85 (m, 12H), 2.85-2.42 (m, AB, 2H), 2.15-1.85 (8×s, 24H), 1.03 (d, 3H).

### Step 3

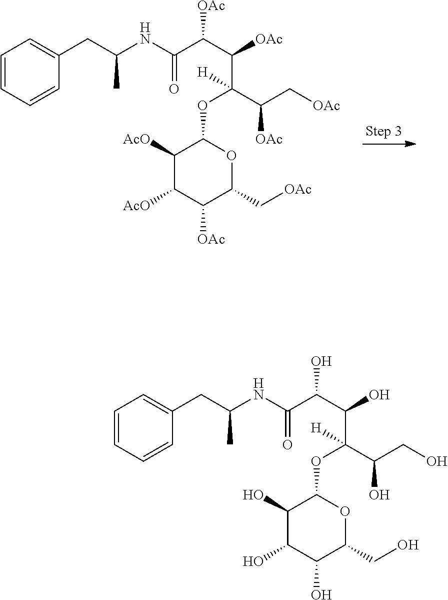 [SMILES: CC(=O)OC[C@H]1O[C@@H](O[C@@H]([C@H](OC(C)=O)[C@@H](OC(C)=O)C(=O)N[C@@H](C)Cc2ccccc2)[C@@H](COC(C)=O)OC(C)=O)[C@H](OC(C)=O)[C@@H](OC(C)=O)[C@H]1OC(C)=O ; C[C@@H](Cc1ccccc1)NC(=O)[C@H](O)[C@@H](O)[C@H](O[C@@H]1O[C@H](CO)[C@H](O)[C@H](O)[C@H]1O)[C@H](O)CO]

### (2R,3R,4R,5R)-2,3,5,6-tetrahydroxy-N—((S)-1-phenylpropan-2-yl)-4-(((2S,3R,4S,5R,6R)-3,4,5-trihydroxy-6-(hydroxymethyl)tetrahydro-2H-pyran-2-yl)oxy)hexanamide

(2R,3R,4S,5R)-6-oxo-6-(((S)-1-phenylpropan-2-yl)amino)-3-(((2S,3R,4S,5S,6R)-3,4,5-triacetoxy-6-(acetoxymethyl)tetrahydro-2H-pyran-2-yl)oxy)hexane-1,2,4,5-tetrayl tetraacetate (254 mg, 0.31 mmol) was dissolved in methanol (20 mL) and sodium methoxide (16.9 mg, 0.313 mmol) was added. The reaction mixture was stirred overnight and then neutralised with DOWEX H⁺ resin. The solution was concentrated to give product which was dissolved in ethanol, filtered through an SCX cartridge, re-concentrated and dried to give (2R,3R,4R,5R)-2,3,5,6-tetrahydroxy-N—((S)-1-phenylpropan-2-yl)-4-(((2S,3R,4S,5R,6R)-3,4,5-trihydroxy-6-(hydroxymethyl)tetrahydro-2H-pyran-2-yl)oxy)hexanamide (85 mg).

¹H NMR (MeOD): δ 7.07 (m, 5H), 4.42 (d, 1H), 4.27 (d, 1H), 4.15 (dd, 1H), 4.08 (m, 1H), 3.85-3.26 (m, 11H), 2.87-2.42 (m, AB, 2H), 1.02 (d, 3H).

MS: m/z=498.0 [M+Na]⁺.

### Example 6

### Step 1

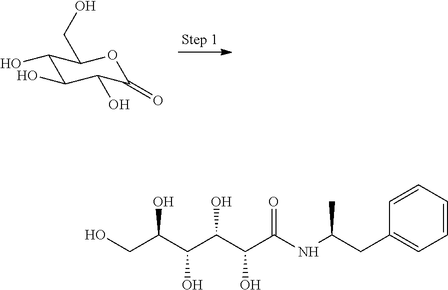 [SMILES: O=C1OC(CO)C(O)C(O)C1O ; C[C@@H](Cc1ccccc1)NC(=O)[C@H](O)[C@@H](O)[C@H](O)[C@H](O)CO]

### (2R,3S,4R,5R)-2,3,4,5,6-pentahydroxy-N—((S)-1-phenylpropan-2-yl)hexanamide

D-(+)-Gluconic acid δ-lactone (242 mg, 1.36 mmol) was dissolved in methanol and (S)-amphetamine hemisulphate (250 mg, 1.36 mmol) was added. Diisopropylethylamine (0.69 g, 6.78 mmol) was added and the reaction mixture was heated overnight at reflux. The reaction mixture was passed through an SCX column, eluting with methanol, to remove amphetamine and DIPEA. The residue was partitioned between water (50 mL) and ethyl acetate (50 mL). Repeated extraction with ethyl acetate gave some product. Extraction was continued with ethyl acetate, drying and evaporating fractions to assess their content. All fractions containing product (by ¹H NMR) were combined and concentrated. The product was re-crystallised from ethyl acetate (6 mL) and the mother liquors removed via syringe. The solid products were dissolved in methanol and concentrated to dryness (2 hours at 40° C.) to give (2R,3S,4R,5R)-2,3,4,5,6-pentahydroxy-N—((S)-1-phenylpropan-2-yl)hexanamide (120 mg).

¹H NMR (MeOD): δ 7.15 (m, 5H), 4.08 (m, 2H), 3.99 (m, 1H), 3.70 (d, 1H), 3.66 (d, 1H), 3.65-3.45 (m, 3H), 2.90-2.50 (m, AB, 2H), 1.00 (d, 3H).

MS: m/z=314.2 [M+Na]⁺.

### Example 7

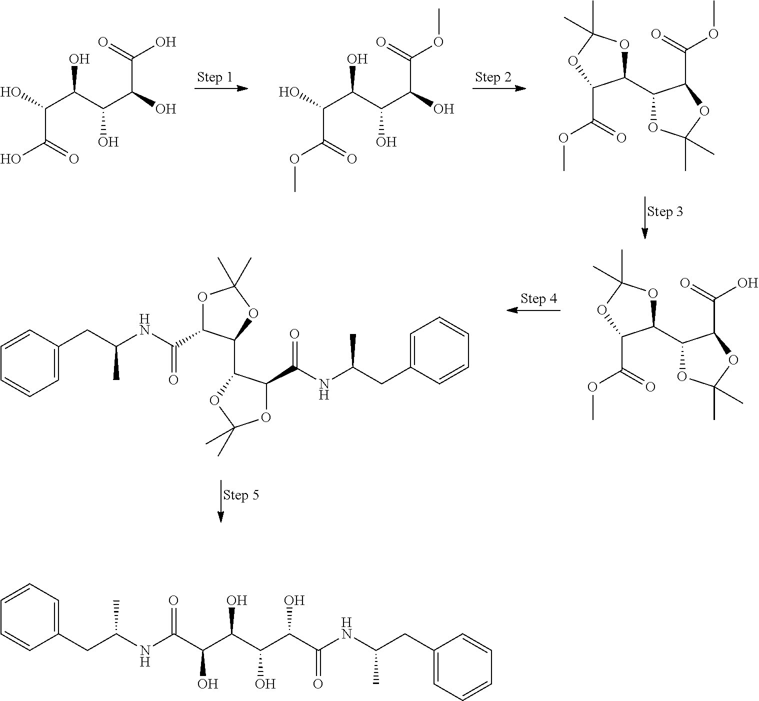 [SMILES: O=C(O)[C@@H](O)[C@H](O)[C@H](O)[C@@H](O)C(=O)O ; COC(=O)[C@@H](O)[C@H](O)[C@H](O)[C@@H](O)C(=O)OC ; COC(=O)[C@H]1OC(C)(C)O[C@@H]1[C@@H]1OC(C)(C)O[C@H]1C(=O)OC ; C[C@@H](Cc1ccccc1)NC(=O)[C@H]1OC(C)(C)O[C@@H]1[C@@H]1OC(C)(C)O[C@H]1C(=O)N[C@@H](C)Cc1ccccc1 ; COC(=O)[C@@H]1OC(C)(C)O[C@H]1[C@H]1OC(C)(C)O[C@@H]1C(=O)O ; C[C@@H](Cc1ccccc1)NC(=O)[C@@H](O)[C@H](O)[C@H](O)[C@@H](O)C(=O)N[C@@H](C)Cc1ccccc1]

### Step 1

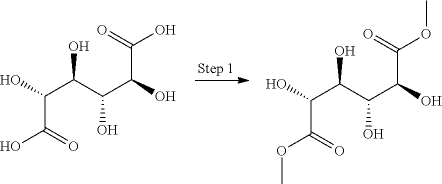 [SMILES: O=C(O)[C@@H](O)[C@H](O)[C@H](O)[C@@H](O)C(=O)O ; COC(=O)[C@@H](O)[C@H](O)[C@H](O)[C@@H](O)C(=O)OC]

### (2R,3S,4R,5S)-Dimethyl 2,3,4,5-tetrahydroxyhexanedioate

(2R,3S,4R,5S)-2,3,4,5-tetrahydroxyhexanedioic acid (10.0 g, 47.6 mmol) was suspended in methanol (193 mL) and concentrated sulphuric acid (2.54 mL, 47.6 mmol) was added. The reaction was heated at reflux for 54 hours. The reaction was cooled, a precipitate formed and it was stirred at room temperature for 36 hours and then at 0° C. overnight. The product was collected by filtration, washed with water and dried under vacuum to give (2R,3S,4R,5S)-dimethyl 2,3,4,5-tetrahydroxyhexanedioate (11.33 g).

¹H NMR (CDCl₃): δ 4.96 (d, 2H), 4.84 (m, 2H), 4.32 (d, 2H), 3.88 (m, 2H), 3.62 (s, 6H).

### Step 2

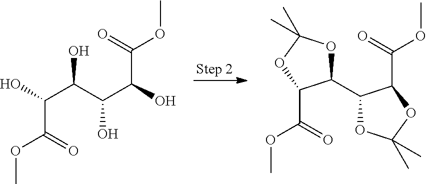 [SMILES: COC(=O)[C@@H](O)[C@H](O)[C@H](O)[C@@H](O)C(=O)OC ; COC(=O)[C@H]1OC(C)(C)O[C@@H]1[C@@H]1OC(C)(C)O[C@H]1C(=O)OC]

### (4R,4′S,5S,5′R)-Dimethyl 2,2,2′,2′-tetramethyl-[4,4′-bi(1,3-dioxolane)]-5,5′-dicarboxyl-ate

(2R,3S,4R,5S)-Dimethyl 2,3,4,5-tetrahydroxyhexanedioate (11.33 g 47.6 mmol) was dissolved in acetone (100 mL). 2,2-dimethoxypropane (58.5 mL, 49.54 g, 0.476 mol) and camphorsulfonic acid (2.21 g, 9.51 mmol) was added and the reaction mixture was heated at reflux until a solution formed (ca. 1 hour). The reaction was cooled and basified with K₂CO₃ (aq., 2 M, 100 mL) causing a colour change from pink to yellow. The acetone and dimethoxypropane was removed under a vacuum and the remaining aqueous phase was extracted with chloroform (2×100 mL). The chloroform layer was dried (MgSO₄) and evaporated in vacuo. The resulting solid was slurried in MeOH (60 mL) and the product collected by filtration to give (4R,4′S,5S,5′R)-dimethyl 2,2,2′,2′-tetramethyl-[4,4′-bi(1,3-dioxolane)]-5,5′-dicarboxyl-ate (5.63 g).

¹H NMR (CDCl₃): δ 4.65 (m, 2H), 4.42 (m, 2H), 3.83 (s, 6H), 1.51 (s, 6H), 1.42 (s, 6H).

### Step 3

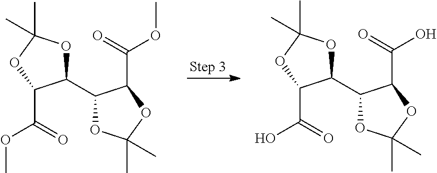

### (4R,4′S,5S,5′R)-2,2,2′,2′-Tetramethyl-[4,4′-bi(1,3-dioxolane)]-5,5′-dicarboxylic acid

(4R,4′S,5S,5′R)-dimethyl 2,2,2′,2′-tetramethyl-[4,4′-bi(1,3-dioxolane)]-5,5′-dicarboxylate (5.63 g, 7.85 mmol) was suspended in water (60 mL) and solid sodium hydroxide (1.57 g, 39.3 mmol) was added. The reaction mixture was heated at 60° C. overnight. A solution formed slowly over the first 2 hours. The reaction mixture was poured into iced 1M HCl (aq., 100 mL) and extracted with ethyl acetate (2×100 m mL). The ethyl acetate layer was dried (MgSO₄) and concentrated in vacuo to give (4R,4′S,5S,5′R)-2,2,2′,2′-tetramethyl-[4,4′-bi(1,3-dioxolane)]-5,5′-dicarboxylic acid (2.28 g).

¹H NMR (d6-DMSO): δ 13.20 (bs, 2H), 4.46 (m, 2H), 4.42 (m, 2H), 1.42 (s, 6H), 1.35 (s, 6H).

### Step 4

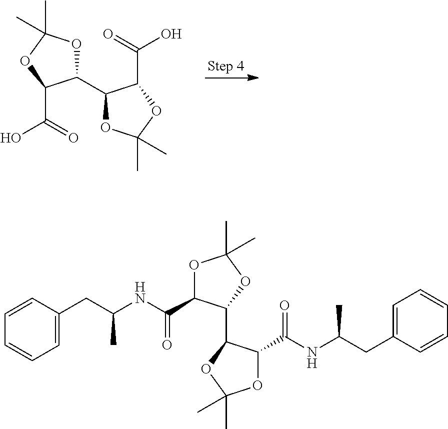 [SMILES: CC1(C)O[C@H]([C@@H]2OC(C)(C)O[C@H]2C(=O)O)[C@@H](C(=O)O)O1 ; C[C@@H](Cc1ccccc1)NC(=O)[C@H]1OC(C)(C)O[C@@H]1[C@@H]1OC(C)(C)O[C@H]1C(=O)N[C@@H](C)Cc1ccccc1]

### (4R,4′S,5S,5′R)-2,2,2′,2′-Tetramethyl-N5,N5′-bis((S)-1-phenylpropan-2-yl)-[4,4′-bi(1,3-dioxolane)]-5,5′-dicarboxamide

(4R,4′S,5S,5′R)-2,2,2′,2′-Tetramethyl-[4,4′-bi(1,3-dioxolane)]-5,5′-dicarboxylic acid (1.00 g, 3.45 mmol) and (S)-amphetamine hemisulfate (1.28 g, 2.94 mmol) were dissolved in DMF (80 mL). HATU (1.70 g, 4.48 mmol) was added and the reaction mixture was stirred at 35° C. for 72 hours. The solvent was removed by evaporation under high vacuum. The residue was partitioned between ethyl acetate (75 mL) and 1M HCl (75 mL). The ethyl acetate layer was washed with further 1M HCl (50 mL), sat. NaHCO_{3(aq)} (50 mL) and brine (50 mL). The ethyl acetate layer was dried (MgSO₄) and concentrated in vacuo to give an orange oil. Column chromatography (SiO₂, eluting with ethyl acetate/petroleum ether 1:4 v/v) gave (4R,4′S,5S,5′R)-2,2,2′,2′-tetramethyl-N5,N5′-bis((S)-1-phenylpropan-2-yl)-[4,4′-bi(1,3-dioxolane)]-5,5′-dicarboxamide (0.81 g).

¹H NMR (CDCl₃): δ 7.25 (m, 10H), 6.62 (m, 1H), 6.51 (m, 1H), 4.67 (dd, 2H), 4.46 (dd, 1H), 4.28 (m, 3H), 2.98-2.60 (m, 4H), 1.38 (d, 6H), 1.40-1.10 (m, 12H).

### Step 5

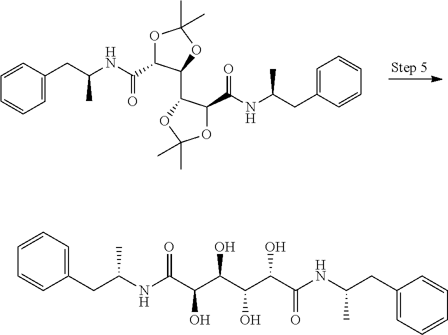 [SMILES: C[C@@H](Cc1ccccc1)NC(=O)[C@H]1OC(C)(C)O[C@@H]1[C@@H]1OC(C)(C)O[C@H]1C(=O)N[C@@H](C)Cc1ccccc1 ; C[C@@H](Cc1ccccc1)NC(=O)[C@@H](O)[C@H](O)[C@H](O)[C@@H](O)C(=O)N[C@@H](C)Cc1ccccc1]

### (2R,3S,4R,5S)-2,3,4,5-Tetrahydroxy-N1,N6-bis((S)-1-phenylpropan-2-yl)hexanediamide

(4R,4′S,5S,5′R)-2,2,2′,2′-Tetramethyl-N5,N5′-bis((S)-1-phenylpropan-2-yl)-[4,4′-bi(1,3-dioxolane)]-5,5′-dicarboxamide (0.81 g, 1.54 mmol) was suspended in TFA (13.2 mL) and water (0.70 mL) and stirred overnight to give a solution. The solvent was removed by evaporation and the product was slurried in diethyl ether (50 mL). The product was collected by filtration, washed with ether (2×25 mL) and dried in a vacuum oven at 40° C. This gave (2R,3S,4R,5S)-2,3,4,5-tetrahydroxy-N1,N6-bis((S)-1-phenylpropan-2-yl)hexanediamide (0.53 g).

¹H NMR (d6-DMSO): δ 7.43 (d, 2H), 7.27 (m, 10H), 5.10 (bs, 4H), 4.11 (d, 2H), 4.05 (m, 2H), 3.75 (s, 2H), 2.88-2.50 (m, 4H), 1.97 (d, 3H), 1.04 (d, 3H).

MS: m/z=467.1 [M+Na]⁺.

### Example 8

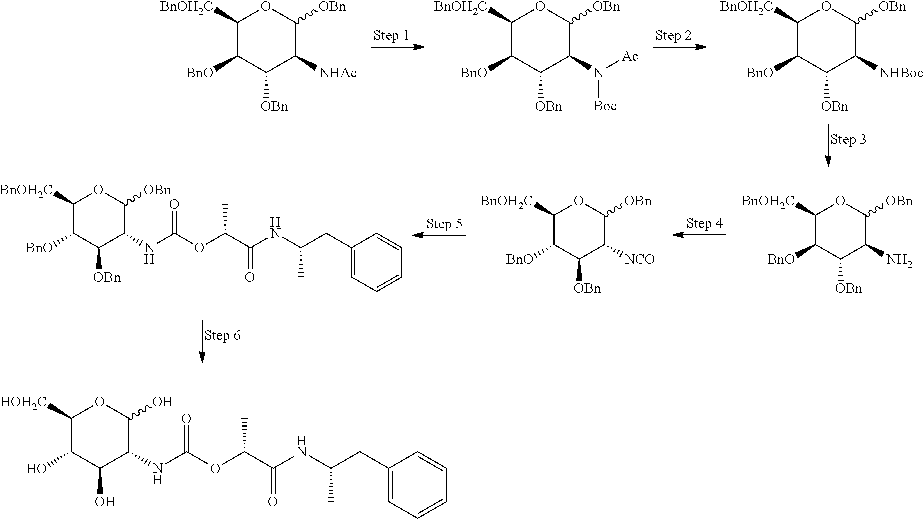

### Step 1

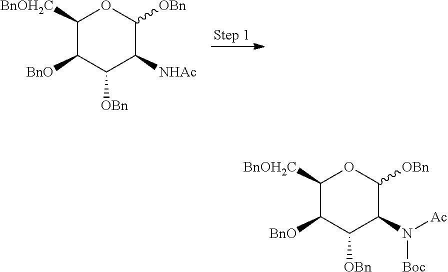

### tert-Butyl N-acetyl-N-[(3S,4S,5R,6R)-2,4,5-tris(benzyloxy)-6-(benzyloxy)methyl]oxan-3-yl]carbamate

To a dry THF (60 mL) solution of N-[(3S,4S,5R,6R)-2,4,5-tris(benzyloxy)-6-[(benzyloxy)methyl]oxan-3-yl]acetamide (4.0 g, 6.88 mmol) and 4-(dimethylamino) pyridine (0.084 g, 0.688 mmol) was added di-tert-butyl dicarbonate (7.50 g, 34.4 mmol) and the reaction mixture was heated at 60° C. for 18 hours. The reaction mixture was concentrated in vacuo to a brown oil (13.5 g). Purification by column chromatography (SiO₂, eluting with an ethyl acetate-petroleum ether, 1:1) afforded tert-butyl N-acetyl-N-[(3S,4S,5R,6R)-2,4,5-tris(benzyloxy)-6-(benzyloxy)methyl]oxan-3-yl]carbamate (5.5 g).

¹H NMR (CDCl₃): δ 7.29 (m, 20H), 5.96 (d, 1H), 5.89-5.52 (m, 9H), 3.92 (m, 1H), 3.80-3.61 (m, 4H), 2.28 (s, 3H), 1.41 (s, 9H).

### Step 2

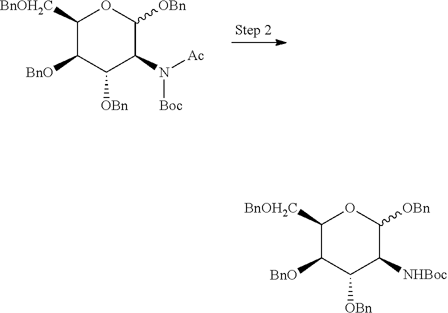

### tert-Butyl N-[(3S,4S,5R,6R)-2,4,5-tris(benzyloxy)-6-[(benzyloxy)methyl]oxan-3-yl]carbamate

To a methanol (25 mL) solution of tert-butyl N-acetyl-N-[(3S,4S,5R,6R)-2,4,5-tris(benzyloxy)-6-[(benzyloxy)methyl]oxan-3-yl]carbamate (5.5 g, 8.07 mmol) was added sodium methoxide (0.011 g, 0.202 mmol). After 18 hours the white precipitate was filtered and washed with cold methanol (10 mL) and the solid was dried in vacuo to afford a tert-butyl N-[(3S,4S,5R,6R)-2,4,5-tris(benzyloxy)-6-[(benzyloxy)methyl]oxan-3-yl]carbamate (3.65 g).

¹H NMR (DMSO-d6): δ 7.45-7.10 (m, 20H), 4.85-4.49 (m, 9H), 3.83-3.59 (m, 7H), 1.40 (s, 9H).

### Step 3

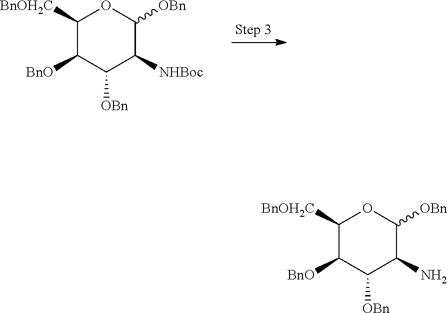

### (3S,4S,5R,6R)-2,4,5-Tris(benzyloxy)-6-[(benzyloxy)methyl]oxan-3-amine

To an acetic acid (20 mL) solution of tert-butyl N-[(3S,4S,5R,6R)-2,4,5-tris(benzyloxy)-6-[(benzyloxy)methyl]oxan-3-yl]carbamate (3.65 g, 5.71 mmol) was added water (4 mL). Trifluoroacetic acid (0.651 g, 5.71 mmol) was added and the reaction mixture was stirred at ambient temperature for 18 hours. The reaction mixture was concentrated in vacuo and then purified by passing through an ion-exchange resin (Porapak-Rxn CX 3×60 cc, eluent 2M NH₃/methanol) to afford (3S,4S,5R,6R)-2,4,5-tris(benzyloxy)-6-[(benzyloxy)methyl]oxan-3-amine (2.65 g).

¹H NMR (CDCl₃): δ 7.45-7.16 (m, 20H), 5.05 (d, 1H), 4.83-4.62 (m, 8H), 3.92-3.65 (m, 6H), 1.62 (bs, 2H).

### Step 4

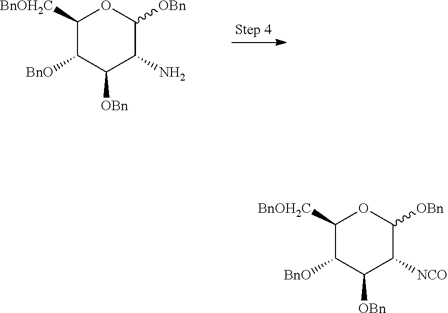

### (3R,4R,5S,6R)-2,4,5-Tris(benzyloxy)-6-[(benzyloxy)methyl]-3-isocyanatooxane

To a water (25 mL) solution of NaHCO₃ (3.64 g, 43.4 mmol) was added (3R,4R,5S,6R)-2,4,5-tris(benzyloxy)-6-[(benzyloxy)methyl]oxan-3-amine (1.95 g, 3.61 mmol) dissolved in toluene (9 mL). The resulting mixture was cooled to 0° C., and phosgene (20% solution) (7 mL) was added in one portion. The solution was stirred at room temperature for 3 hours. During this time the suspension became a biphasic clear solution. The reaction mixture was then separated, the aqueous layer was further extracted with diethyl ether (3×20 mL), and the combined organics were dried over magnesium sulfate and concentrated in vacuo to afford (3R,4R,5S,6R)-2,4,5-tris(benzyloxy)-6-[(benzyloxy)methyl]-3-isocyanatooxane (2.0 g).

IR (thin film) νₘₐₓ (cm⁻¹): 2248 (m)

### Step 5

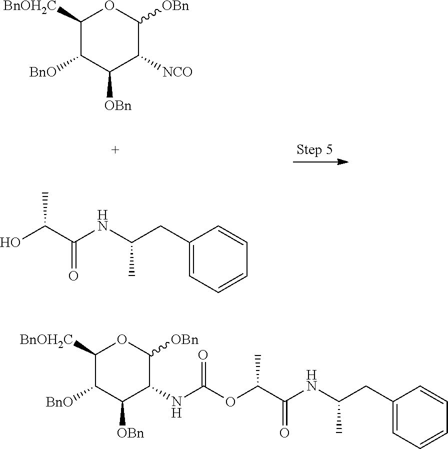

### (1R)-1-{[(2S)-1-Phenylpropan-2-yl]carbamoyl}ethylN-[(3R,4R,5S,6R)-2,4,5-tris(benzyloxy)-6-[(benzyloxy)methyl]oxan-3-yl]carbamate

To a dry THF (17 mL) solution of (2R)-2-hydroxy-N-[(2S)-1-phenylpropan-2-yl]propanamide (0.50 g, 2.41 mmol) was added potassium tert-butoxide (0.271 g, 2.41 mmol) and the reaction mixture was stirred for 10 minutes before the addition of a dry THF (17 mL) solution of (3R,4R,5S,6R)-2,4,5-tris(benzyloxy)-6-[(benzyloxy)methyl]-3-isocyanatooxane (2.00 g, 3.54 mmol). After stirring the reaction mixture for 18 hours at ambient temperature it was concentrated in vacuo to an orange semi-solid (2.4 g), which was purified by column chromatography (ethyl acetate-petroleum ether, 1:2) to afford (1R)-1-{[(2S)-1-phenylpropan-2-yl]carbamoyl}ethyl N-[(3R,4R,5S,6R)-2,4,5-tris(benzyloxy)-6-[(benzyloxy)methyl]oxan-3-yl]carbamate (0.45 g).

¹H NMR (CDCl₃): δ 7.32-6.99 (m, 25H), 5.76 (d, 1H), 5.15-4.40 (m, 10H), 4.16-3.88 (m, 2H), 3.80-3.59 (m, 6H), 2.86-2.55 (m, 2H), 1.34 (d, 3H), 0.86 (d, 3H).

### Step 6

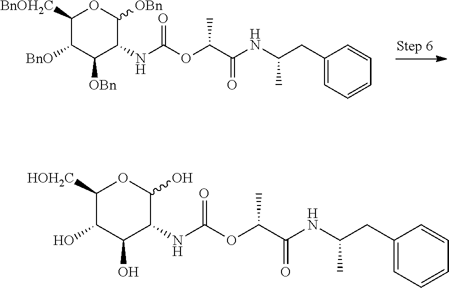

### (1R)-1-{[(2S)-1-Phenylpropan-2-yl]carbamoyl}ethylN-[(3R,4R,5S,6R)-2,4,5-trihydroxy-6-(hydroxymethyl)oxan-3-yl]carbamate

To a THF (4 mL)/water (1.95 mL)/formic acid (0.28 mL) solution of (1R)-1-{[(2S)-1-phenylpropan-2-yl]carbamoyl}ethyl N-[(3R,4R,5S,6R)-2,4,5-tris(benzyloxy)-6-[(benzyl oxy)methyl]oxan-3-yl]carbamate (0.488 g, 0.631 mmol) under an inert atmosphere was added Pd(OH)₂/C and the reaction mixture was put under 1 atmosphere pressure of H₂. The reaction mixture was then stirred at 23° C. for 18 hours. The reaction mixture was filtered through celite and concentrated in vacuo to a yellow oil (0.65 g). The crude product was purified by preparative HPLC (Phenomenex Kinetex C18 column using a gradient of 10-100% acetonitrile/0.1% trifluoroacetic acid in water as eluent) to afford (1R)-1-{[(2S)-1-phenylpropan-2-yl]carbamoyl}ethyl N-[(3R,4R,5S,6R)-2,4,5-trihydroxy-6-(hydroxymethyl)oxan-3-yl]carbamate (0.061 g).

¹H NMR (CDCl₃): δ 7.27-7.10 (m, 5H), 4.80 (d, 1H), 3.70-3.20 (m, 8H), 2.95-2.62 (m, 3H), 1.15-0.99 (m, 6H).

MS: m/z=434.92 [M+Na]⁺

### Example 9

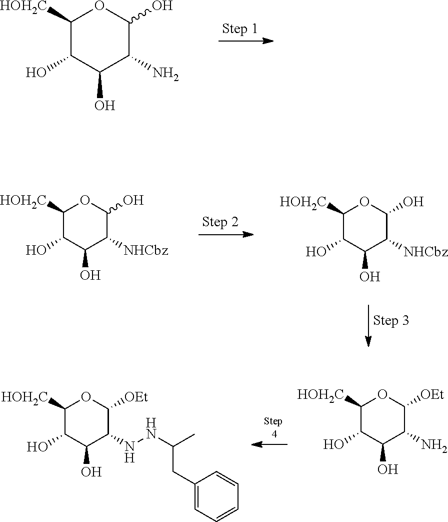 [SMILES: N[C@H]1C(O)O[C@H](CO)[C@@H](O)[C@@H]1O ; O=C(N[C@H]1C(O)O[C@H](CO)[C@@H](O)[C@@H]1O)OCc1ccccc1 ; O=C(N[C@@H]1[C@@H](O)[C@H](O)[C@@H](CO)O[C@@H]1O)OCc1ccccc1 ; CCO[C@H]1O[C@H](CO)[C@@H](O)[C@H](O)[C@H]1NNC(C)Cc1ccccc1 ; CCO[C@H]1O[C@H](CO)[C@@H](O)[C@H](O)[C@H]1N]

(2R,3S,4R,5R,6S)-6-ethoxy-2-(hydroxymethyl)-5-[2-(1-phenyl propan-2-yl)hydrazin-1-yl]oxane-3,4-diol (0.096 g) was prepared in a manner analogous to Example 2 and by the reaction scheme shown above.

¹H NMR (CDCl₃): δ 7.22 (m, 5H), 4.83 (d, 1H), 3.70-3.19 (m, 9H), 2.88 (m, 2H), 2.71 (m, 1H), 1.15 (d, 3H), 1.02 (t, 3H).

MS: m/z=362.93 [M+Na]⁺

### Example 10

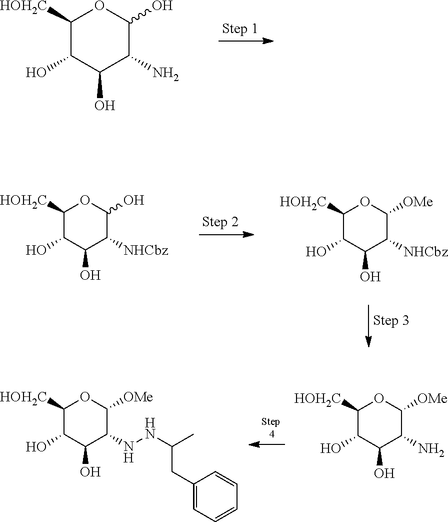 [SMILES: N[C@H]1C(O)O[C@H](CO)[C@@H](O)[C@@H]1O ; O=C(N[C@H]1C(O)O[C@H](CO)[C@@H](O)[C@@H]1O)OCc1ccccc1 ; CO[C@H]1O[C@H](CO)[C@@H](O)[C@H](O)[C@H]1NC(=O)OCc1ccccc1 ; CO[C@H]1O[C@H](CO)[C@@H](O)[C@H](O)[C@H]1NNC(C)Cc1ccccc1 ; CO[C@H]1O[C@H](CO)[C@@H](O)[C@H](O)[C@H]1N]

(2R,3S,4R,5R,6S)-6-methoxy-2-(hydroxymethyl)-5-[2-(1-phenyl propan-2-yl)hydrazin-1-yl]oxane-3,4-diol (0.245 g) was prepared in a manner analogous to Example 2 and by the reaction scheme shown above.

¹H NMR (CDCl₃): δ 7.48-7.25 (m, 5H), 4.86 (d, 1H), 3.92-3.70 (m, 3H), 3.60-3.48 (m, 2H), 3.42 (s, 3H), 3.21-3.02 (m, 2H), 2.84-2.68 (m, 2H), 1.25 (apparent dd, 3H).

MS: m/z=327.00 [M+Na]⁺

### Example 11

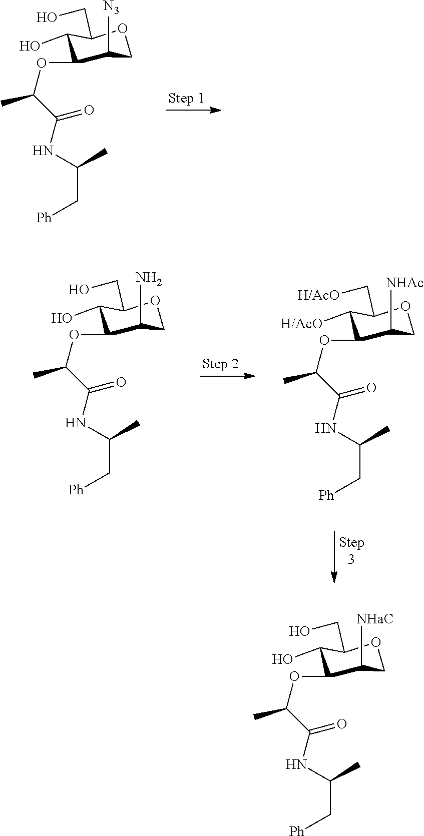

### Step 1

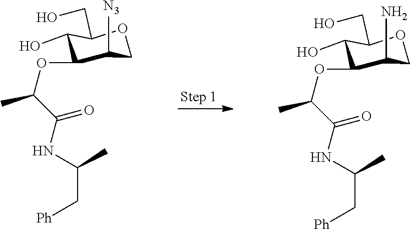

### (R)-2-(((2R,3S,4R,5R)-5-Amino-3-hydroxy-2-(hydroxymethyl)tetrahydro-2H-pyran-4-yl)oxy)-N—((S)-1-phenylpropan-2-yl)propanamide

(R)-2-(((2R,3S,4R,5R)-5-Azido-3-hydroxy-2-(hydroxymethyl)tetrahydro-2H-pyran-4-yl)oxy)-N—((S)-1-phenylpropan-2-yl)propanamide (0.21 g, 0.581 mmol) was dissolved in pyridine (20 mL), water (4 mL) and triethylamine (0.05 mL). Propene dithiol (0.584 mL, 0.629 g, 5.81 mmol) was added and the reaction mixture was stirred overnight after which time the solvent and reagents were removed under high vacuum. Proton NMR showed the presence of pure product and thiol derivatives. The product was used directly in the next step.

¹H NMR (D₂O): 7.21 (m, 5H), 4.11 (m, 1H), 3.94 (q, 1H), 3.80-3.67 (m, 2H), 3.47 (dd, 1H), 3.53-3.38 (m, 2H), 3.12 (m, 2H), 2.88 (dd, 1H), 2.63-2.46 (m, 2H), 1.20-1.08 (2d, 6H).

### Step 2

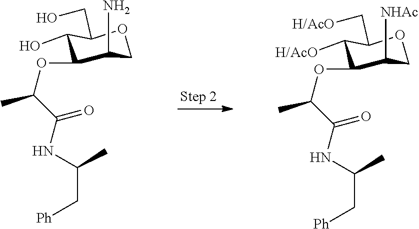

### (2R,3S,4R,5R)-5-Acetamido-2-(acetoxymethyl)-4-(((R)-1-oxo-1-(((S)-1-phenylpropan-2-yl)amino)propan-2-yl)oxy)tetrahydro-2H-pyran-3-yl acetate

(R)-2-(((2R,3S,4R,5R)-5-amino-3-hydroxy-2-(hydroxymethyl)tetrahydro-2H-pyran-4-yl)oxy)-N—((S)-1-phenylpropan-2-yl)propanamide (0.21 g, 0.582 mmol) was dissolved in pyridine (20 mL) and acetic anhydride (1.18 g, 11.63 mmol) was added. The solution was stirred for 30 minutes and was then concentrated to dryness under high vacuum. The residue was dissolved in dichloromethane (50 mL) and washed with brine (10 mL). The organic phase was dried (MgSO₄) and concentrated to dryness. The product was purified, by flash column chromatography (SiO₂, eluting with dichloromethane and then dichloromethane/methanol 4:1 v/v). Evaporating the required fractions gave an oil which was used directly in the next step.

### Step 3

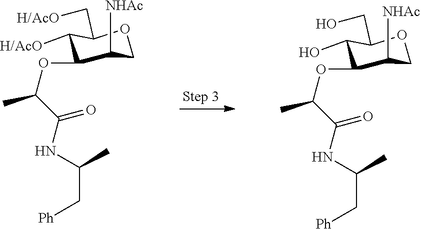

### (R)-2-(((2R,3S,4R,5R)-5-Acetamido-3-hydroxy-2-(hydroxymethyl)tetrahydro-2H-pyran-4-yl)oxy)-N—((S)-1-phenylpropan-2-yl)propanamide

(2R,3S,4R,5R)-5-Acetamido-2-(acetoxymethyl)-4-(((R)-1-oxo-1-(((S)-1-phenylpropan-2-yl)amino)propan-2-yl)oxy)tetrahydro-2H-pyran-3-yl acetate from Step 2 was dissolved in methanol (10 mL) and sodium methoxide (23 mg, 0.418 mmol) was added. After 2 hours at room temperature the reaction mixture was neutralised with DOWEX H⁺ resin and concentrated to dryness. The product was freeze dried from acetonitrile/water (1:1 v/v 5 mL) to give (R)-2-(((2R,3S,4R,5R)-5-acetamido-3-hydroxy-2-(hydroxymethyl)tetrahydro-2H-pyran-4-yl)oxy)-N—((S)-1-phenylpropan-2-yl)propanamide (0.165 g).

¹H NMR (D₂O): 7.15 (m, 5H), 4.13 (m, 1H), 4.06 (m, 1H), 3.96 (q, 1H), 3.78-3.60 (m, 3H), 3.56-3.40 (m, 3H), 3.20 (m, 1H), 2.90-2.58 (m, 2H), 1.93 (s, 3H), 1.20 (d, 3H), 0.97 (d, 3H).

MS: m/z=416.86 [M+Na]⁺.

### Example 12

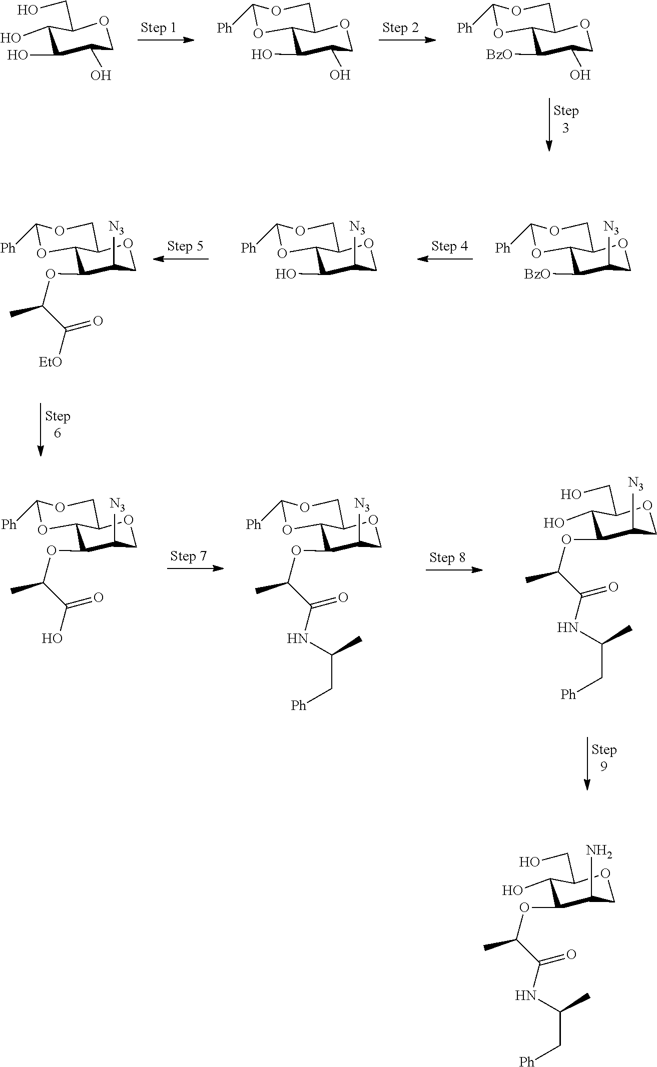

(R)-2-(((2R,3S,4R,5R)-5-amino-3-hydroxy-2-(hydroxymethyl)tetrahydro-2H-pyran-4-yl)oxy)-N—((S)-1-phenylpropan-2-yl)propanamide (0.050 g) was prepared in a manner analogous to Example 11 and by the reaction scheme shown above.

¹H NMR (D₂O): 7.21 (m, 5H), 4.11 (m, 1H), 3.94 (q, 1H), 3.80-3.67 (m, 2H), 3.47 (dd, 1H), 3.53-3.38 (m, 2H), 3.12 (m, 2H), 2.88 (dd, 1H), 2.63-2.46 (m, 2H), 1.20-1.08 (2d, 6H).

MS: m/z=374.98 [M+Na]⁺.

### Example 13

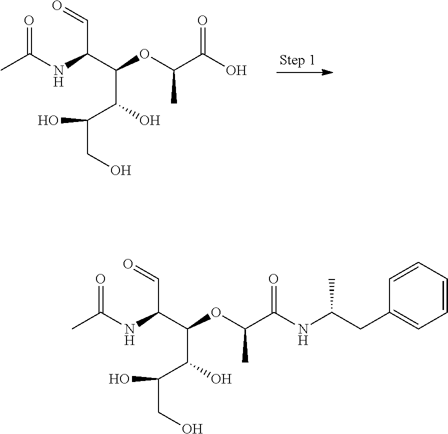 [SMILES: CC(=O)N[C@@H](C=O)[C@@H](O[C@H](C)C(=O)O)[C@H](O)[C@H](O)CO ; CC(=O)N[C@@H](C=O)[C@@H](O[C@H](C)C(=O)N[C@H](C)Cc1ccccc1)[C@H](O)[C@H](O)CO]

### Step 1

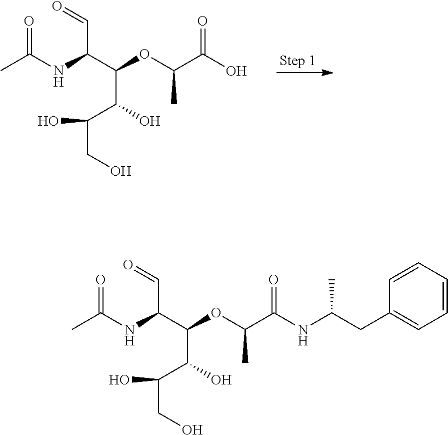 [SMILES: CC(=O)N[C@@H](C=O)[C@@H](O[C@H](C)C(=O)O)[C@H](O)[C@H](O)CO ; CC(=O)N[C@@H](C=O)[C@@H](O[C@H](C)C(=O)N[C@H](C)Cc1ccccc1)[C@H](O)[C@H](O)CO]

### (2R)-2-{[(2R,3R,4R,5R)-2-Acetamido-4,5,6-trihydroxy-1-oxohexan-3-yl]oxy}-N-[(2R)-1-phenylpropan-2-yl]propanamide

To a dichloromethane (5.6 mL) solution of (2R)-2-{[(2R,3R,4R,5R)-2-acetamido-4,5,6-trihydroxy-1-oxohexan-3-yl]oxy}propanoic acid (0.519 g, 1.77 mmol) was added (R)-amphetamine hemisulphate (0.215 g, 0.797 mmol). To the resultant suspension was added 1-[bis(dimethylamino)methylene]-1H-1,2,3-triazolo[4,5-b]pyridinium 3-oxid hexafluorophosphate (0.748 g, 1.95 mmol) and N,N-diisopropylethylamine (0.686 g, 5.31 mmol). The reaction was stirred at ambient temperature for 20 hours and then concentrated in vacuo to a yellow oil. THF (5.5 mL) was added and the mixture was re-concentrated. THF (5.5 mL) was added and the solution was stirred for 30 minutes, by which point no solid had precipitated. The solution was re-concentrated to afford a yellow oil (2.2 g). This was purified by column chromatography (SiO₂, eluting with an isocratic solvent gradient of H₂O-THF, 0.5:100), which yielded a yellow oil (0.87 g). This was further purified by preparative HPLC (Waters Atlantis T3 C18 column using a gradient of 10-100% methanol/10 mM NH₄HCO₃ in water as eluent) to afford (2R)-2-{[(2R,3R,4R,5R)-2-acetamido-4,5,6-trihydroxy-1-oxohexan-3-yl]oxy}-N-[(2R)-1-phenylpropan-2-yl]propanamide (0.254 g).

¹H NMR (MeOD): δ 7.25 (m, 5H), 5.24 (d, 1H), 4.45 (q, 1H), 4.13 (m, 2H), 3.87-3.40 (m, 7H), 3.92 (dd, 1H), 3.71 (dd, 1H), 2.03 (s, 3H), 1.31 (d, 3H), 1.12 (d, 3H).

MS: m/z=432.97 [M+Na]⁺.

### Example 14

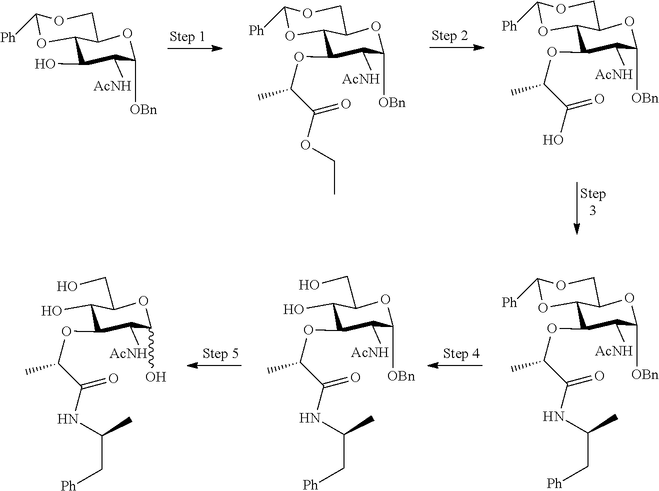

### Step 1

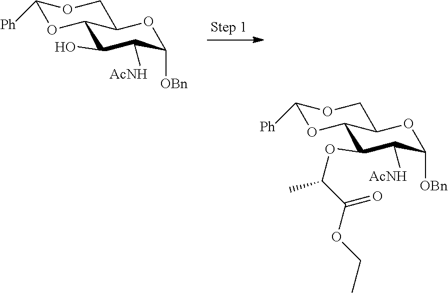

### (S)-ethyl 2-(((2R,4aR,6S,7R,8R,8aS)-7-Acetamido-6-(benzyloxy)-2-phenylhexahydro pyrano[3,2-d][1,3]dioxin-8-yl)oxy)propanoate

N-((2R,4aR,6S,7R,8R,8aS)-6-(Benzyloxy)-8-hydroxy-2-phenylhexahydropyrano[3,2-d][1,3]dioxin-7-yl)acetamide (5.00 g, 12.4 mmol) was dissolved in tetrahydrofuran (100 mL) and DMF (20 mL) and sodium hydride (1.52 g, 37.6 mmol) was added at −20° C. After 30 minutes at −20° C., (R)-ethyl 2-(((trifluoromethyl)sulfonyl)oxy)propanoate (9.40 g, 37.6 mmol) was added, over 30 minutes at −20° C., as a solution in THF (10 mL). Stirring was continued overnight and the reaction mixture was partitioned between tert-butylmethyl ether (20 mL) and brine (50 mL). An emulsion formed so ethyl acetate, THF and MeOH were added to break the emulsion. The organic layer was separated, dried (MgSO₄) and concentrated at reduced pressure to give a crude product. The product was purified by flash column chromatography (SiO₂, eluting with acetone/petroleum ether 10:40 v/v) to give (S)-ethyl 2-(((2R,4aR,6S,7R,8R,8aS)-7-acetamido-6-(benzyloxy)-2-phenylhexahydro pyrano[3,2-d][1,3]dioxin-8-yl)oxy)propanoate (3.35 g).

¹H NMR (CDCl₃): δ 7.40-7.19 (m, 10H), 5.90 (d, 1H), 5.39 (s, 1H), 4.89 (d, 1H), 4.63 (d, 1H), 4.38 (d, 1H), 4.30-4.03 (m, 3H), 3.85-3.57 (m, 5H), 3.50-3.35 (m, 1H), 1.89 (s, 3H), 1.24 (d, 3H), 0.89 (t, 3H).

MS: m/z=500.1 [M+H]⁺.

### Step 2

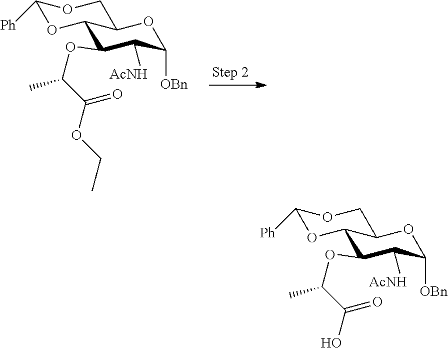

### (S)-2-(((2R,4aR,6S,7R,8R,8aS)-7-Acetamido-6-(benzyloxy)-2-phenylhexahydro pyrano[3,2-d][1,3]dioxin-8-yl)oxy)propanoic acid

(S)-Ethyl 2-(((2R,4aR,6S,7R,8R,8aS)-7-acetamido-6-(benzyloxy)-2-phenylhexahydro pyrano[3,2-d][1,3]dioxin-8-yl)oxy)propanoate (3.31 g, 6.63 mmol) was dissolved in methanol (15 mL), water (15 mL) and THF (20 mL). Potassium hydroxide (1.49 g, 26.5 mmol) was added and the mixture was stirred for 3 hours at room temperature. The methanol was evaporated and the reaction partitioned between dichloromethane (100 mL) and 10% citric acid_{(aq)} (50 mL). The dichloromethane layer was washed with water (50 mL) then brine (50 ml), dried (MgSO₄) and concentrated at reduced pressure to give (S)-2-(((2R,4aR,6S,7R,8R,8aS)-7-acetamido-6-(benzyloxy)-2-phenylhexahydro pyrano[3,2-d][1,3]dioxin-8-yl)oxy)propanoic acid (3.12 g).

¹H NMR (CDCl₃): δ 7.45-7.27 (m, 10H), 5.66 (d, 1H), 5.58 (s, 1H), 4.83 (d, 1H), 4.76 (d, 1H), 4.52 (d, 1H), 4.31 (q, 1H), 4.26 (dd, 1H), 3.75 (m, 3H), 3.40 (m, 2H), 1.96 (s, 3H), 1.37 (d, 3H).

### Step 3

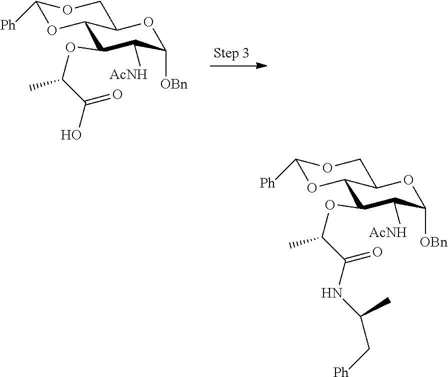

### (S)-2-(((2R,4aR,6S,7R,8R,8aS)-7-Acetamido-6-(benzyloxy)-2-phenylhexahydro pyrano[3,2-d][1,3]dioxin-8-yl)oxy)-N—((S)-1-phenylpropan-2-yl)propanamide

Batch 1: (S)-2-(((2R,4aR,6S,7R,8R,8aS)-7-acetamido-6-(benzyloxy)-2-phenylhexahydro pyrano[3,2-d][1,3]dioxin-8-yl)oxy)propanoic acid (0.64 g, 1.36 mmol) was dissolved in dichloromethane (20 mL) and (S)-amphetamine hemisulfate (0.38 g, 1.08 mmol) was added followed by HATU (0.52 g, 1.36 mmol) and diisopropylethylamine (0.53 g, 4.07 mmol). The reaction mixture was stirred for 2 days at 25° C. and was combined with batch 2.

Batch 2: (S)-2-(((2R,4aR,6S,7R,8R,8aS)-7-acetamido-6-(benzyloxy)-2-phenylhexahydro pyrano[3,2-d][1,3]dioxin-8-yl)oxy)propanoic acid (0.30 g, 0.64 mmol) was dissolved in dichloromethane (20 mL) and (S)-amphetamine hemisulfate (0.18 g, 0.48 mmol) was added followed by HATU (0.24 g, 0.64 mmol) and diisopropylethylamine (0.53 g, 4.07 mmol). The reaction mixture was stirred for 2 days at 25° C. and was combined with batch 1.

Batches 1 and 2 were combined, diluted with dichloromethane (50 mL), washed with water (25 mL), NaHCO_{3(aq)} (25 mL) and brine (25 mL). The organic phase was dried (MgSO₄) and concentrated under reduced pressure. The product was crystallised from methanol, collected by filtration and dried to give ((S)-2-(((2R,4aR,6S,7R,8R,8aS)-7-acetamido-6-(benzyloxy)-2-phenylhexahydropyrano[3,2-d][1,3]dioxin-8-yl)oxy)-N—((S)-1-phenylpropan-2-yl)propanamide (0.37 g).

¹H NMR (CDCl₃): δ 7.45-6.95 (m, 15H), 6.82 (d, 1H), 5.50 (d, 1H), 5.44 (s, 1H), 4.71 (d, 1H), 4.67 (d, 1H), 4.42 (d, 1H), 4.36-4.15 (m, 2H), 4.03-3.72 (m, 3H), 3.71-3.44 (m, 3H), 2.18 (d, 2H), 1.86 (s, 3H), 1.21 (d, 3H), 0.59 (d, 3H).

### Step 4

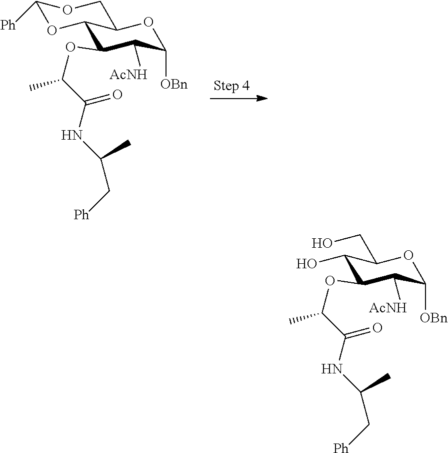

### (S)-2-(((2S,3R,4R,5S,6R)-3-Acetamido-2-(benzyloxy)-5-hydroxy-6-(hydroxymethyl)tetrahydro-2H-pyran-4-yl)oxy)-N—((S)-1-phenylpropan-2-yl)propanamide

(S)-2-(((2R,4aR,6S,7R,8R,8aS)-7-Acetamido-6-(benzyloxy)-2-phenylhexahydro pyrano[3,2-d][1,3]dioxin-8-yl)oxy)-N—((S)-1-phenylpropan-2-yl)propanamide (0.37 g, 0.63 mmol) was suspended in methanol (10 mL) and para-toluenesulfonic acid (0.12 g, 0.63 mmol) was added. After 1.5 hours at 70° C., the reaction mixture was cooled and triethylamine was added to quench the reaction. The solvent was evaporated and the product purified by flash column chromatography (SiO₂, eluting with dichloromethane/methanol 1:9 v/v) to give (S)-2-(((2S,3R,4R,5S,6R)-3-acetamido-2-(benzyloxy)-5-hydroxy-6-(hydroxymethyl)tetrahydro-2H-pyran-4-yl)oxy)-N—((S)-1-phenylpropan-2-yl)propanamide (0.28 g).

¹H NMR (CDCl₃): δ 7.37-7.05 (m, 10H), 5.61 (d, 1H), 4.71 (d, 1H), 4.63 (d, 1H), 4.37 (d, 1H), 4.25 (m, 2H), 4.02 (q, 1H), 3.70 (m, 2H), 3.51-3.30 (m, 6H), 3.18 (d, 1H), 2.42 (m, 1H), 1.92 (s, 3H), 1.22 (d, 3H), 1.12 (d, 3H).

### Step 5

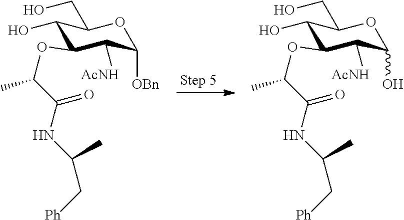

### (2S)-2-(((3R,4R,5S,6R)-3-Acetamido-2,5-dihydroxy-6-(hydroxymethyl)tetrahydro-2H-pyran-4-yl)oxy)-N—((S)-1-phenylpropan-2-yl)propanamide

(S)-2-(((2S,3R,4R,5S,6R)-3-Acetamido-2-(benzyloxy)-5-hydroxy-6-(hydroxymethyl)tetrahydro-2H-pyran-4-yl)oxy)-N—((S)-1-phenylpropan-2-yl)propanamide (0.28 g, 0.56 mmol) was dissolved in tetrahydrofuran (8 mL), water (8 mL) and formic acid (1 mL). Palladium hydroxide 20% on carbon (0.39 g, 0.56 mmol) was added and the reaction mixture was put under 1 atmosphere pressure of H₂ overnight. The catalyst was filtered off and the solvent evaporated. The product was purified by preparative HPLC (Waters Xbridge C18 column using 10-100% acetonitrile/0.1% formic acid in water as eluent). Freeze drying gave (2S)-2-(((3R,4R,5S,6R)-3-acetamido-2,5-dihydroxy-6-(hydroxymethyl)tetrahydro-2H-pyran-4-yl)oxy)-N—((S)-1-phenylpropan-2-yl)propan-amide.

¹H NMR (D₂O): δ 7.22 (m, 5H), 5.01 (d, 0.7H), 4.66 (d, 0.3H), 4.10-3.77 (m, 3H), 3.75-3.52 (m, 3H), 3.47-3.18 (m, 3H), 2.85-2.54 (m, 2H), 1.92 (s, 3H), 1.07 (2t, 6H).

MS: m/z=432.84 [M+Na]⁺.

### Example 15

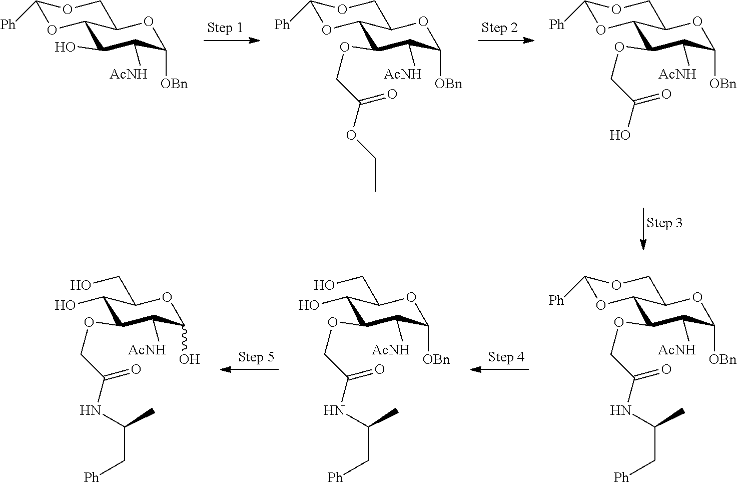

2-(((3R,4R,5S,6R)-3-acetamido-2,5-dihydroxy-6-(hydroxymethyl)tetrahydro-2H-pyran-4-yl)oxy)-N—((S)-1-phenylpropan-2-yl)acetamide (65 mg) was prepared in a manner analogous to Example 14 and by the reaction scheme shown above.

¹H NMR (D₂O): δ 7.30-7.00 (m, 5H), 5.10 (d, 0.7H), 4.52 (d, 0.3H), 4.10-3.48 (m, 6H), 3.47-3.13 (m, 4H), 2.80-2.50 (m, 2H), 1.85 (s, 3H), 1.06, d, 3H).

MS: m/z=397.1 [M+H]⁺.

### Example 16

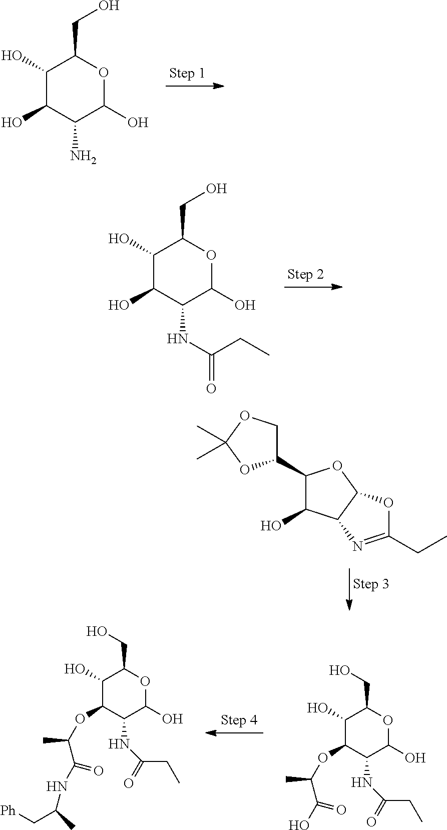

### Step 1

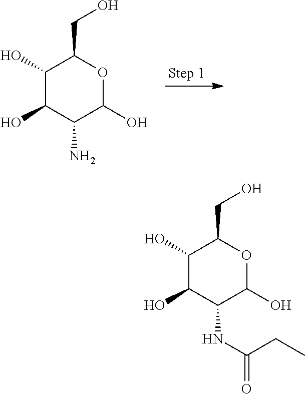 [SMILES: N[C@H]1C(O)O[C@H](CO)[C@@H](O)[C@@H]1O ; CCC(=O)N[C@H]1C(O)O[C@H](CO)[C@@H](O)[C@@H]1O]

### N-[(3R,4R,5S,6R)-2,4,5-Trihydroxy-6-(hydroxymethyl)oxan-3-yl]propanamide

To a methanol (90 mL) solution of sodium (1.1 g, 47.9 mmol) was added (3R,4R,5S,6R)-3-amino-6-(hydroxymethyl)oxane-2,4,5-triol hydrochloride (10 g, 46.4 mmol) and the reaction mixture was stirred at 15-20° C. for 10 minutes. The resultant precipitate was filtered off and washed with cold methanol (40 mL). Propionic anhydride (9.05 g, 69.6 mmol) was immediately added to the filtrates and the mixture was stirred at 15-20° C. for 1 hour. A precipitate formed that was filtered off and washed successively with cold methanol (30 mL) and cold diethyl ether (30 mL) before drying in vacuo to afford N-[(3R,4R,5S,6R)-2,4,5-trihydroxy-6-(hydroxymethyl)oxan-3-yl]propanamide (2.68 g).

¹H NMR (MeOD-d⁴): δ 5.13 (d, 1H), 3.90-3.67 (m, 4H), 3.57-3.38 (m, 2H), 2.30 (q, 2H), 1.14 (t, 3H).

### Step 2

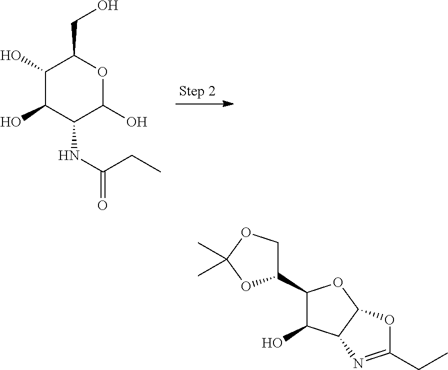 [SMILES: CCC(=O)N[C@H]1C(O)O[C@H](CO)[C@@H](O)[C@@H]1O ; CCC1=N[C@H]2[C@@H](O1)O[C@H]([C@H]1COC(C)(C)O1)[C@@H]2O]

### (3aR,5S,6R,6aR)-5-[(4R)-2,2-Dimethyl-1,3-dioxolan-4-yl]-2-ethyl-3a,5,6,6a-tetrahydro-ofuro[3,2-d]oxazol-6-ol

N-[(3R,4R,5S,6R)-2,4,5-trihydroxy-6-(hydroxymethyl)oxan-3-yl]propanamide (2.60 g, 11.1 mmol) was suspended in dry acetone (26 mL) and FeCl₃ (3.59 g, 22.1 mmol) was added in one portion, which caused a small exotherm. The mixture was heated at reflux for 25 minutes, which caused all the solid to dissolve and the colour of the reaction mixture to become brown, and then the reaction was cooled for 10 minutes The reaction was then cooled to 0° C. and diethylamine (8.80 mL, 6.22 g, 85.1 mmol) was added, which gave a brown precipitate, additional acetone (31 mL) was added and then sodium carbonate (aq) (5.10 g dissolved in water (32 mL)). The mixture was then concentrated in vacuo to a volume of ca. 30 mL. The aqueous phase was then extracted with TBME (10×20 mL) and the combined organics were dried over MgSO₄, filtered and concentrated in vacuo to give (3aR,5S,6R,6aR)-5-[(4R)-2,2-dimethyl-1,3-dioxolan-4-yl]-2-ethyl-3a,5,6,6a-tetrahydrofuro[3,2-d]oxazol-6-ol (1.35 g).

¹H NMR (CDCl₃): δ 6.21 (d, 1H), 4.51-4.42 (m, 2H), 4.38-4.28 (m, 1H), 4.18 (dd, 1H), 4.01 (dd, 1H), 3.75 (dd, 1H), 2.57 (d, 1H), 2.36 (q, 2H), 1.44 (s, 3H), 1.38 (s, 3H), 1.21 (t, 3H).

### Step 3

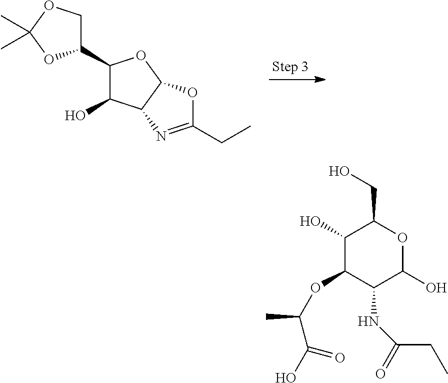 [SMILES: CCC1=N[C@H]2[C@@H](O1)O[C@H]([C@H]1COC(C)(C)O1)[C@@H]2O ; CCC(=O)N[C@H]1C(O)O[C@H](CO)[C@@H](O)[C@@H]1O[C@H](C)C(=O)O]

### (2R)-2-{[(3R,4R,5S)-2,5-Dihydroxy-6-(hydroxymethyl)-3-propanamidooxan-4-yl]oxy}propanoic acid

Sodium hydride (60% dispersion in mineral oil, 1.05 g, 26.2 mmol) was dissolved in dry dioxane (13 mL) and then to this solution was added (3aR,5S,6R,6aR)-5-[(4R)-2,2-dimethyl-1,3-dioxolan-4-yl]-2-ethyl-3a,5,6,6a-tetrahydrofuro[3,2-d]oxazol-6-ol (1.35 g, 5.25 mmol) dissolved in dry dioxane (15 mL). After stirring the reaction mixture at room temperature for 5 minutes the reaction mixture was heated to 80-85° C. for 30 minutes. The reaction mixture was then cooled to 65° C. and (S)-2-chloropropionic acid (1.42 g, 13.1 mmol) diluted with dry dioxane (3 mL) was added dropwise, which lead to formation of a precipitate and gas evolution. The reaction mixture was stirred at 65° C. for a further 1 hour. The reaction was stirred at room temperature overnight and then cooled to 15-25° C. and the pH adjusted to 8 by the dropwise addition of 2M HCl. The reaction mixture was concentrated in vacuo, and then partitioned between water (10 mL) and DCM (25 mL) and the pH further adjusted to pH 3 by the dropwise addition of 2M HCl. The mixture was separated and the aqueous phase further extracted with DCM (3×25 mL). The combined organic extracts were dried over MgSO₄, filtered and concentrated in vacuo to an orange oil (1.42 g). To this residue was added 80% AcOH_{(aq)} (20 mL) and the reaction mixture was stirred for 20 hours at room temperature. Analysis by LCMS showed starting material and intermediate remaining; therefore the reaction was heated at 35° C. for 10 hours and then stirred at room temperature for 72 hours. Analysis by LCMS then showed no starting material or intermediate, so the reaction was concentrated in vacuo. Water was added (2×30 mL) and removed in vacuo, then ethyl acetate was added (4×20 mL) and concentrated in vacuo to give (2R)-2-{[(3R,4R,5S)-2,5-dihydroxy-6-(hydroxymethyl)-3-propanamidooxan-4-yl]oxy}propanoic acid (0.97 g).

¹H NMR (MeOD-d4): δ 5.39 (d, 1H), 4.72-4.55 (m, 1H), 3.82-3.41 (m, 6H), 2.27 (q, 2H), 1.42 (d, 3H), 1.14 (t, 3H).

### Step 4

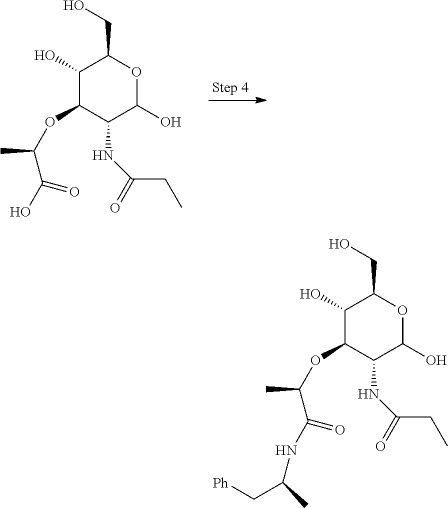 [SMILES: CCC(=O)N[C@H]1C(O)O[C@H](CO)[C@@H](O)[C@@H]1O[C@H](C)C(=O)O ; CCC(=O)N[C@H]1C(O)O[C@H](CO)[C@@H](O)[C@@H]1O[C@H](C)C(=O)N[C@@H](C)Cc1ccccc1]

### (2R)-2-[(3R,4R,5S)-2,5-dihydroxy-6-(hydroxymethyl)-3-(propanoylamino)tetrahydro pyran-4-yl]oxy-N-[(1S)-1-methyl-2-phenyl-ethyl]propanamide

(S)-Amphetamine hemisulfate (0.30 g, 0.82 mmol) was slurried in DCM (5.6 mL) and added to (2R)-2-[(3R,4R,5S)-2,5-dihydroxy-6-(hydroxymethyl)-3-(propanoyl amino)tetrahydropyran-4-yl]oxypropanoic acid (0.52 g, 1.69 mmol). To the resultant suspension was added HATU (0.71 g, 1.86 mmol) and finally diisopropylethylamine (0.88 mL, 0.66 g, 5.07 mmol). The reaction was left to stir for 20 hours at 5-12° C. and then concentrated in vacuo at 35° C. to give a yellow oil. Purification by column chromatography (SiO₂, eluting with an THF-water, 100:0.5) afforded a yellow oil that was further purified by preparative HPLC (Waters XBridge C18 column using a gradient of 10-100% acetonitrile/0.1% trifluoroacetic acid in water as eluent). Freeze drying gave (2R)-2-[(3R,4R,5S)-2,5-dihydroxy-6-(hydroxymethyl)-3-(propanoylamino)-tetrahydropyran-4-yl]oxy-N-[(1S)-1-methyl-2-phenyl-ethyl]propanamide (0.22 g).

¹H NMR (MeOD-d4): δ 8.14 (d, 1H), 7.31-7.14 (m, 5H), 5.18 (d, 1H), 4.28-4.02 (m, 2H), 3.93-3.53 (m, 4H), 3.49-3.35 (m, 1H), 2.89-2.65 (m, 2H), 2.22 (qd, 2H), 1.28 (d, 3H), 1.16-1.07 (m, 6H).

MS: m/z=446.97 [M+Na]⁺.

### Example 17

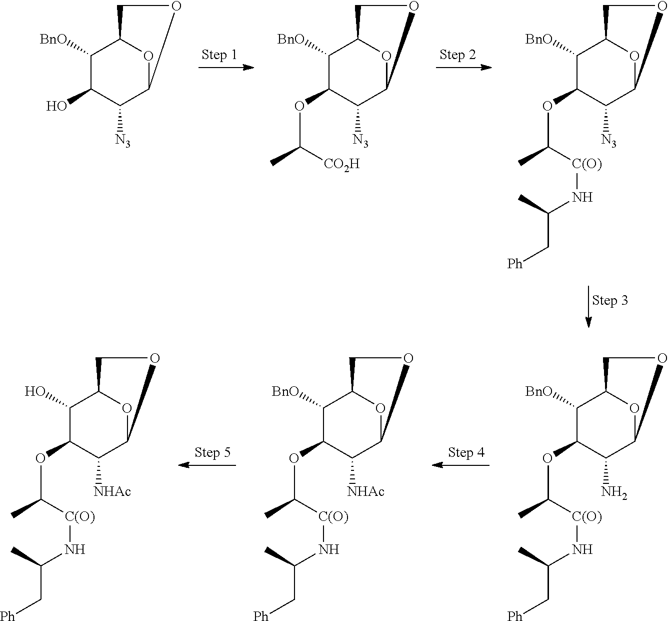

### Step 1

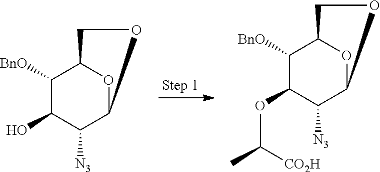 [SMILES: [N-]=[N+]=N[C@H]1[C@@H]2OC[C@@H](O2)[C@@H](OCc2ccccc2)[C@@H]1O ; C[C@@H](O[C@@H]1[C@@H](N=[N+]=[N-])[C@@H]2OC[C@@H](O2)[C@H]1OCc1ccccc1)C(=O)O]

### (2R)-2-{[(2S,3R,4R)-4-Azido-2-(benzyloxy)-6,8-dioxabicyclo[3.2.1]octan-3-yl]oxy}propanoic acid

To a stirred dry dioxane (35 mL) of (2S,3R,4R)-4-azido-2-(benzyloxy)-6,8-dioxabicyclo[3.2.1]octan-3-ol solution (1.98 g, 7.14 mmol) at 45° C. was added NaH 60% dispersion in mineral oil (1.9 g, 47.1 mmol). The mixture was kept at 45° C. for 10 minutes and then the temperature was decreased to ambient temperature for the addition of chloropropionic acid (1.86 g, 17.1 mmol), which caused an effervescence but no exotherm, and then the stirring was continued at 90° C. for 2 hours. The solvent was then evaporated in vacuo and water (5 mL) was carefully added to quench the excess sodium hydride. The resultant aqueous solution was then extracted with ethyl acetate/Petrol (1:1), to remove the mineral oil. The filtrates were then cooled to 0° C. and acidified to pH 3 by the careful addition of 2M HCl. The resultant precipitate was extracted with DCM (3×15 mL) and the combined organic extracts were dried over MgSO₄, filtered and concentrated in vacuo to give a yellow oil (2.95 g). Purification by column chromatography (SiO₂, eluting with chloroform/acetone, 3:1) afforded (2R)-2-{[(2S,3R,4R)-4-azido-2-(benzyloxy)-6,8-dioxabicyclo[3.2.1]octan-3-yl]oxy}propanoic acid (1.98 g).

¹H NMR (CDCl₃): δ 10.31 (broad s, 1H), 7.44-7.32 (m, 5H), 5.56 (s, 1H), 4.75 (d, 1H), 4.68 (s, 1H), 4.67 (d, 1H), 4.01 (t, 2H), 3.79-3.72 (m, 1H), 3.65 (s, 1H), 3.38 (s, 2H), 1.42 (d, 3H).

### Step 2

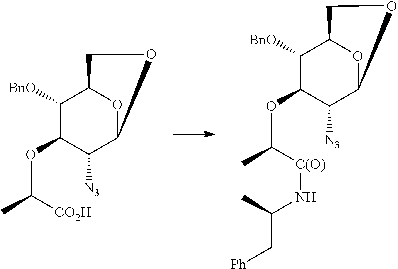

### (2R)-2-{[(2S,3R,4R)-4-Azido-2-(benzyloxy)-6,8-dioxabicyclo[3.2.1]octan-3-yl]oxy}-N-[(2R)-1-phenylpropan-2-yl]propanamide

(S)-Amphetamine hemisulfate (1.37 g, 3.72 mmol) was added to a DCM (26 mL) solution of (2R)-2-{[(2S,3R,4R)-4-azido-2-(benzyloxy)-6,8-dioxabicyclo[3.2.1]octan-3-yl]oxy}propanoic acid (2.6 g, 7.44 mmol) followed by HATU (3.15 g, 8.19 mmol) and diisopropylethyamine (1.92 g, 14.9 mmol). The reaction mixture was stirred for 44 hours at 25° C. and was then diluted with DCM (30 ml), washed with water (20 mL), NaHCO_{3(aq)} (20 mL), dried (MgSO₄) and concentrated in vacuo to a yellow oil (5.38 g). Purification by column chromatography (SiO₂, eluting with chloroform/acetone, 6:1) afforded (2R)-2-{[(2S,3R,4R)-4-azido-2-(benzyloxy)-6,8-dioxabicyclo[3.2.1]octan-3-yl]oxy}-N-[(2R)-1-phenylpropan-2-yl]propanamide (2.05 g).

¹H NMR (CDCl₃): δ 7.42-7.18 (m, 10H), 7.05 (d, 1H), 5.52 (s, 1H), 4.70 (d, 2H), 4.63-4.60 (m, 1H), 4.28-4.17 (m, 1H), 3.84 (d, 1H), 3.76 (q, 1H), 3.65 (dd, 1H), 3.61-3.57 (m, 1H), 3.36 (s, 1H), 3.00 (s, 1H), 2.88-2.72 (m, 2H), 1.27 (d, 3H), 1.17 (d, 3H).

### Step 3

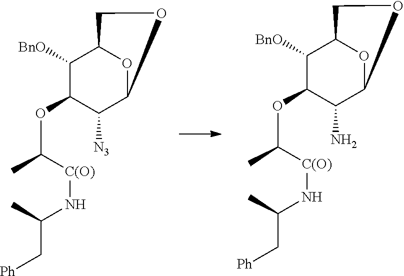

### (2R)-2-{[(2S,3R,4R)-4-Amino-2-(benzyloxy)-6,8-dioxabicyclo[3.2.1]octan-3-yl]oxy}-N-[(2R)-1-phenylpropan-2-yl]propanamide

To a methanol (18 mL) solution of (2R)-2-{[(2S,3R,4R)-4-azido-2-(benzyloxy)-6,8-dioxabicyclo[3.2.1]octan-3-yl]oxy}-N-[(2R)-1-phenylpropan-2-yl]propanamide (1.16 g, 2.49 mmol) was added 10% Pd/C (0.15 g, 0.14 mmol) and the reaction mixture was put under a 1 atmosphere pressure of H₂. The reaction mixture was stirred at 10-15° C. for 18 hours, and the reaction was filtered through celite and the filtrates were concentrated in vacuo to give (2R)-2-{[(2S,3R,4R)-4-amino-2-(benzyloxy)-6,8-dioxabicyclo[3.2.1]octan-3-yl]oxy}-N-[(2R)-1-phenylpropan-2-yl]propanamide (0.98 g).

¹H NMR (CDCl₃): δ 7.41-7.18 (m, 10H), 7.03 (d, 1H), 5.51 (s, 1H), 4.70 (d, 2H), 4.69-4.63 (m, 1H), 4.29-4.17 (m, 1H), 3.85 (d, 1H), 3.76 (q, 1H), 3.66 (dd, 1H), 3.59 (t, 1H), 3.51 (s, 1H), 3.36 (s, 1H), 3.00 (s, 1H), 2.88-2.78 (m, 2H), 1.27 (d, 3H), 1.17 (d, 3H).

### Step 4

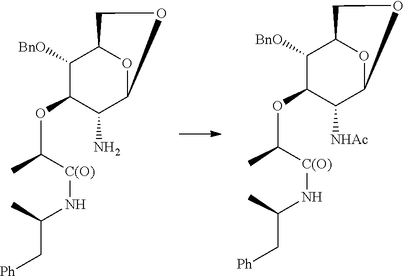

### (2R)-2-{[(2S,3R,4R)-2-(Benzyloxy)-4-acetamido-6,8-dioxabicyclo[3.2.1]octan-3-yl]oxy}-N-[(2R)-1-phenylpropan-2-yl]propanamide

A DCM (7.4 mL) solution of (2R)-2-{[(2S,3R,4R)-4-amino-2-(benzyloxy)-6,8-dioxabicyclo[3.2.1]octan-3-yl]oxy}-N-[(2R)-1-phenylpropan-2-yl]propanamide (0.98 g, 2.23 mmol) was treated with acetic anhydride (7.35 mL, 7.94 g, 35.0 mmol) and the reaction mixture was stirred at room temperature for 22 hours. The volatiles were then removed in vacuo and the residue dissolved into DCM (20 mL) and water (20 mL). The resultant biphasic solution was stirred at ambient temperature for 1 hour and then separated. The organic layer was dried over MgSO₄, filtered and concentrated in vacuo to a colourless oil. Purification by column chromatography (SiO₂, eluting with DCM/acetone, 4:1 afforded (2R)-2-{[(2S,3R,4R)-2-(benzyloxy)-4-acetamido-6,8-dioxabicyclo[3.2.1]octan-3-yl]oxy}-N-[(2R)-1-phenyl propan-2-yl]propanamide (0.97 g).

¹H NMR (CDCl₃): δ 7.42-7.12 (m, 10H), 6.04 (d, 1H), 5.32 (s, 1H), 5.30 (s, 2H), 4.65 (dd, 2H), 4.60 (s, 1H), 4.31-4.20 (m, 1H), 4.01 (dd, 2H), 3.94 (d, 1H), 3.65 (dd, 1H), 3.47 (s, 1H), 2.88-2.73 (m, 2H), 1.93 (s, 3H), 1.30 (d, 3H), 1.17 (d, 3H).

### Step 5

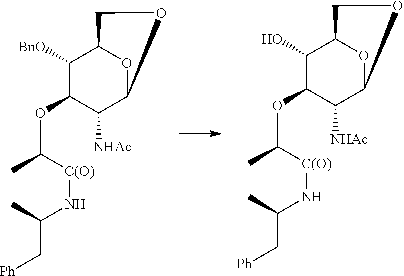

### (2R)-2-[[(2S,3R,4R)-4-acetamido-2-hydroxy-6,8-dioxabicyclo[3.2.1]octan-3-yl]oxy]-N-[(1R)-1-methyl-2-phenyl-ethyl]propanamide

To a methanol (10 mL) solution of (2R)-2-{[(2S,3R,4R)-2-(benzyloxy)-4-acetamido-6,8-dioxabicyclo[3.2.1]octan-3-yl]oxy}-N-[(2R)-1-phenyl propan-2-yl]propanamide (0.97 g, 2.01 mmol) was added 20% Pd(OH)₂/C (0.5 g, 0.71 mmol) and the reaction mixture was put under 1 atmosphere pressure of H₂. After 20 hours the reaction mixture was filtered through celite and the filtrates were concentrated in vacuo to a yellow oil. Purification by preparative HPLC (Waters XBridge C18 column using a gradient of 10-100% acetonitrile/10 mMolar NH₄HCO₃ in water as eluent) afforded (2R)-2-[[(2S,3R,4R)-4-acetamido-2-hydroxy-6,8-dioxabicyclo[3.2.1]octan-3-yl]oxy]-N-[(1R)-1-methyl-2-phenyl-ethyl]propanamide (0.43 g).

¹H NMR (CDCl₃): δ 7.32-7.18 (m, 5H), 6.13 (d, 1H), 5.40 (s, 1H), 4.54 (d, 1H), 4.31-4.19 (m, 1H), 4.06 (q, 1H), 4.03-3.97 (m, 2H), 3.75 (s, 1H), 3.68 (dd, 1H), 3.41 (s, 1H), 2.91-2.73 (m, 3H), 2.02 (s, 3H), 1.34 (d, 3H), 1.18 (d, 3H).

MS: m/z=414.91 [M+Na]⁺.

### Example 18

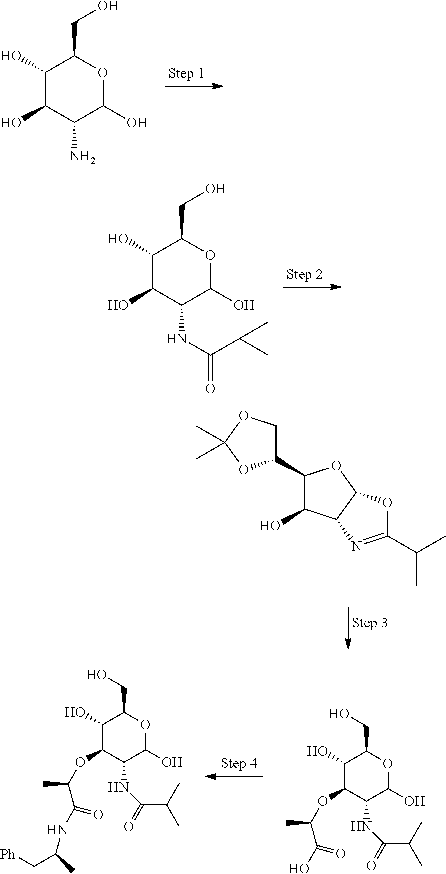

((2R)-2-(((3R,4R,5S,6R)-2,5-dihydroxy-6-(hydroxymethyl)-3-isobutyramidotetrahydro-2H-pyran-4-yl)oxy)-N—((S)-1-phenylpropan-2-yl)propanamide (0.21 g) was prepared in a manner analogous to Example 16 and by the reaction scheme shown above.

¹H NMR (MeOD-d4): δ 7.58 (d, 1H), 7.34-7.14 (m, 5H), 5.18 (d, 0.7H), 4.51 (d, 0.3H), 4.31-4.02 (m, 2H), 3.93-3.53 (m, 5H), 3.49-3.35 (m, 1H), 2.89-2.65 (m, 2H), 2.50 (h, 1H), 1.28 (m, 3H), 1.32-1.07 (m, 9H).

MS: m/z=461.0 [M+Na]⁺.

### Example 19

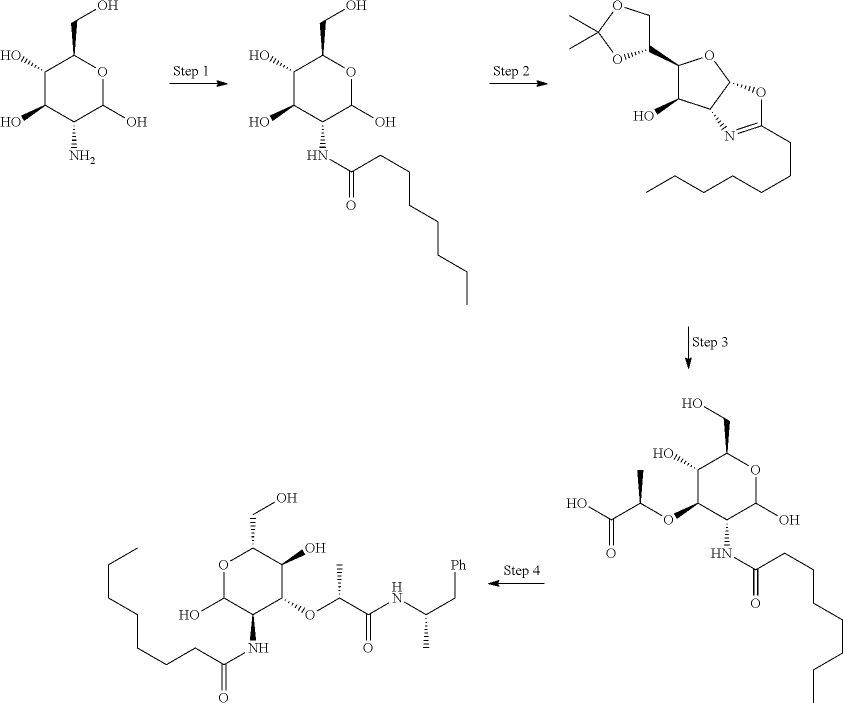 [SMILES: N[C@H]1C(O)O[C@H](CO)[C@@H](O)[C@@H]1O ; CCCCCCCC(=O)N[C@H]1C(O)O[C@H](CO)[C@@H](O)[C@@H]1O ; CCCCCCCC1=N[C@H]2[C@@H](O1)O[C@H]([C@H]1COC(C)(C)O1)[C@@H]2O ; CCCCCCCC(=O)N[C@H]1C(O)O[C@H](CO)[C@@H](O)[C@@H]1O[C@H](C)C(=O)N[C@@H](C)Cc1ccccc1 ; CCCCCCCC(=O)N[C@H]1C(O)O[C@H](CO)[C@@H](O)[C@@H]1O[C@H](C)C(=O)O]

N-((3R,4R,5S,6R)-2,5-dihydroxy-6-(hydroxymethyl)-4-(((R)-1-oxo-1-(((S)-1-phenylpropan-2-yl)amino)propan-2-yl)oxy)tetrahydro-2H-pyran-3-yl)octanamide (0.05 g) was prepared in a manner analogous to Example 16 and by the reaction scheme shown above.

¹H NMR (MeOD-d4): δ 7.34-7.14 (m, 5H), 6.82 (d, 1H), 5.16 (d, 0.7H), 4.46 (d, 0.3H), 4.31-4.02 (m, 2H), 3.93-3.53 (m, 5H), 3.49-3.35 (m, 1H), 3.00 (m, 2H), 2.89-2.65 (m, 2H), 2.20 (m, 2H), 1.73 (m, 2H), 1.30 (m, 8H), 1.20-1.10 (2d, 3H), 0.90 (m, 3H).

MS: m/z=517.01 [M+Na]⁺.

### Example 20

### Pharmacokinetic Parameters of Prodrug and Amphetamine after Oral Dosing of d-Amphetamine or Prodrugs to Male Dogs

### Methodology

Test substances ((d)-amphetamine at 0.5 mg free base equivalents/kg or (d)-amphetamine prodrugs at 0.5 mg d-amphetamine free base equivalents/kg) were administered by oral gavage to groups of 2 male dogs.

Blood samples were taken at various times after administration and submitted to analysis for the parent drug and pro-drug using a qualified LC-MS-MS assay. Pharmacokinetic parameters derived from the plasma analytical data were determined using non-compartmental analysis.

### Results

The data are presented in Table 1 and FIG. 1. Following oral administration, amphetamine was rapidly absorbed with Tₘₐₓ occurring within 1 hour. The pharmacokinetic profile of Compounds 1, 13, 16 and 17 exhibited an amphetamine Tₘₐₓ between 6 and 12 h. In the dog, compounds 1 and 16 showed a relative amphetamine bioavailability above 50%.

Without being bound by any particular theory, this long retention time suggests that the prodrugs were retained to some extent in the gastrointestinal tract and converted to amphetamine in the colon by microflora. The lag time for appearance of amphetamine in the systemic circulation was 2-4 hours for Compounds 1, 13 and 16. The presence of prodrug and absence of amphetamine in the systemic circulation prior to the lag time demonstrated that conversion of absorbed prodrug to the active did not occur.

TABLE 1

| | Parameter | | | |
|---|---|---|---|---|
| | Prodrug | | Amphetamine | |
| | AUC | | AUC | |
| Compound | (hr · ng/mL) | Tₘₐₓ (h) | (hr · ng/mL) | Tₘₐₓ (h) |
| d-Amphetamine | — | — | 1070 | 0.65 |
| 1 | 354 | 1.0 | 522 | 8.0 |
| 13 | 564 | 0.75 | 308 | 12.0 |
| 16 | 273 | 0.5 | 620 | 6.0 |
| 17 | 213 | 0.5 | 26.4 | 9.0 |

Compounds 3-12, 14, 15, 18 and 19 were also tested but showed negligible or very low levels of circulating amphetamine. Compounds 6, 9 and 10 showed a high absorption of prodrug in the upper gastrointestinal tract, therefore, insufficient compound would have been delivered to the colon for conversion to the active by the microbiota. Notably, compounds 5 and 17 showed greater activity in human in vitro studies as discussed below.

### Example 21

### Pharmacokinetic Parameters of Prodrug and Amphetamine after Oral Dosing of d-Amphetamine or Prodrug to Male Cynomolgus Monkeys

### Methodology

Test substances ((d)-amphetamine at 1 mg free base equivalents per kg or d-amphetamine prodrugs at 0.5 mg d-amphetamine free base equivalents/kg) were administered by oral gavage to groups of 5 cynomolgus monkeys.

Blood samples were taken at various times after administration and submitted to analysis for the parent drug and pro-drug using a qualified LC-MS-MS assay. Pharmacokinetic parameters derived from the plasma analytical data were determined using non-compartmental analysis.

### Results

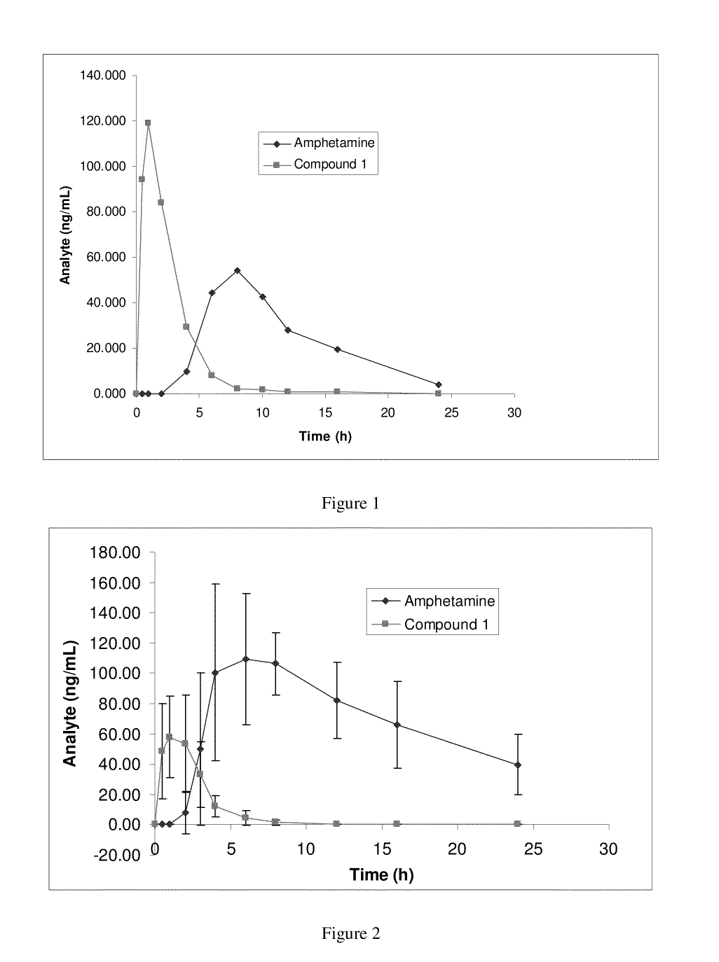

The data are presented in Table 2 and FIG. 2. Following oral administration, amphetamine was rapidly absorbed with Tₘₐₓ occurring within 1 hour. The amphetamine Tₘₐₓ for compound 1 was 6.4 hours which suggested that the prodrug was retained to some extent in the gastrointestinal tract and converted to amphetamine in the colon by microflora. The relative bioavailability was approximately 70%. The lag time for appearance of amphetamine in the systemic circulation was 2-4 hours. The presence of prodrug and absence of amphetamine in the systemic circulation prior to the lag time demonstrated that conversion of absorbed prodrug to the active did not occur.

TABLE 2

| | Parameter | | | |
|---|---|---|---|---|
| | Prodrug | | Amphetamine | |
| | AUC | | AUC | |
| Compound | (hr · ng/mL) | Tₘₐₓ (h) | (hr · ng/mL) | Tmax (h) |
| d-Amphetamine | — | — | 4440 | 2.4 |
| (1 mg/kg) | | | | |
| 1 | 187 | 1.10 | 1630 | 6.40 |

### Example 22

### Pharmacokinetic Parameters of Prodrug and Amphetamine after Intravenous Dosing to Male Cynomolgus Monkeys

### Methodology

Compound 1 (at 0.2 mg d-amphetamine free base equivalents/kg) was administered by oral gavage to 5 cynomolgus monkeys.

Blood samples were taken at various times after administration and submitted to analysis for the parent drug and pro-drug using a qualified LC-MS-MS assay. Pharmacokinetic parameters derived from the plasma analytical data were determined using non-compartmental analysis.

### Results

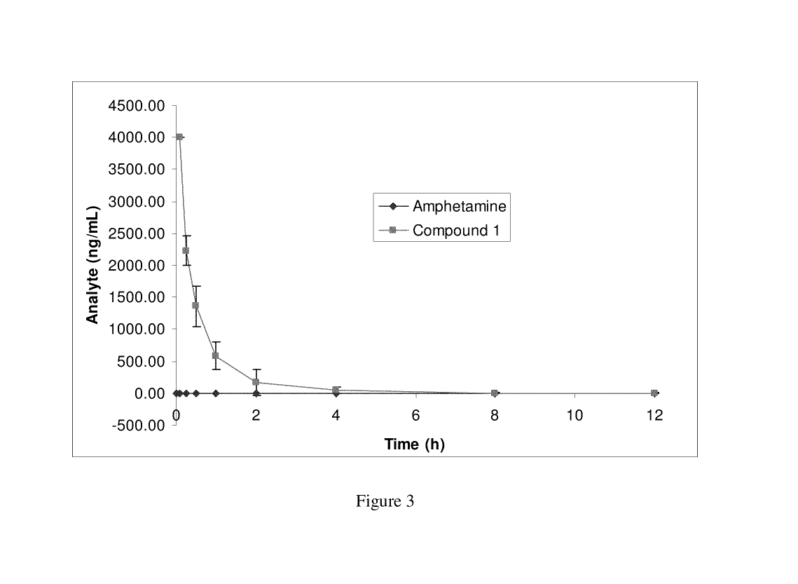

The data are presented in FIG. 3. Following intravenous administration of Compound 1, amphetamine was detected in only 3 out of 5 animals. In these three animals amphetamine was only detected at one time point between 4 and 8 hours at levels near the limit of quantification. This confirms that no significant quantities of Compound 1 are converted to the active amphetamine following systemic exposure, and indicates that the colonic microflora are the site of enzymatic conversion to amphetamine. Without being bound by any particular theory, the inventors theorize that the trace amounts of amphetamine observed in some monkeys at the later time points are due to the biliary excretion of prodrug followed by conversion in the colon.

### Example 23

### Pharmacokinetic Parameters of Amphetamine after Oral Dosing of d-Amphetamine or Compound 1, or Intranasal Dosing of Compound 1 to Male Dogs

### Methodology

Test substances ((d)-amphetamine at 0.5 mg free base equivalents/kg or Compound 1 (a (d)-amphetamine prodrug) at 4 mg d-amphetamine free base equivalents) were administered by oral gavage to groups of 5 male dogs. Compound 1 was also administered to male dogs intranasally using an atomizer. The atomizer delivered 0.1 ml per spray and each dog received 8 sprays (4 per nostril) to deliver a total dose of 4 mg of the active ingredient.

Blood samples were taken at various times after administration and submitted to analysis for the parent drug and pro-drug using a qualified LC-MS-MS assay. Pharmacokinetic parameters derived from the plasma analytical data were determined using non-compartmental analysis.

### Results

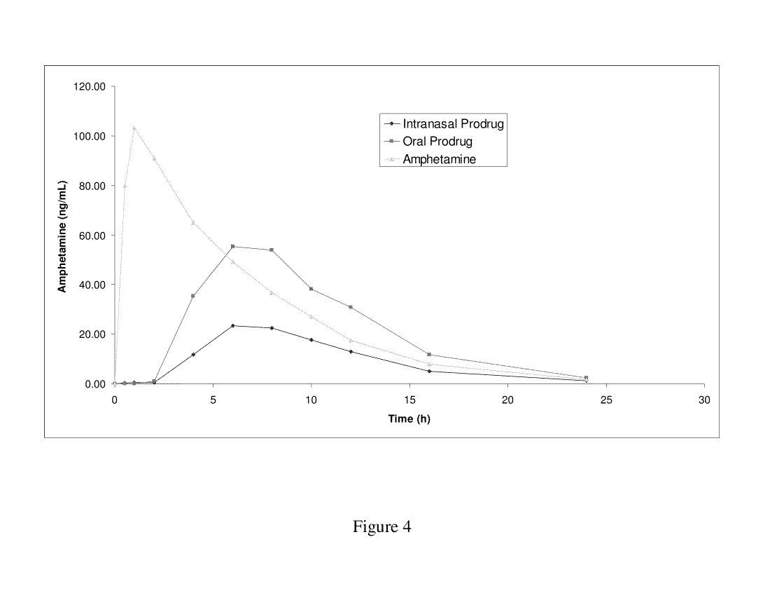

The data are presented in Table 3 (d-amphetamine and Compound 1) and FIG. 4 (Compound 1 only). Following oral administration, amphetamine was rapidly absorbed with Tₘₐₓ occurring within 1 hour. Compound 1 exhibited an amphetamine Tₘₐₓ at 7.2 hours.

The pharmacokinetic profile showed that intranasal administration of the prodrug did not enhance the onset of amphetamine appearance in the systemic circulation. Indeed, the exposure to amphetamine was less than that observed following oral administration of the prodrug with a similar Tₘₐₓ (7.2 h) and lag time. In contrast, the Tₘₐₓ following oral administration of amphetamine was 0.9 h. This suggests that any prodrug absorbed by the intranasal route is not converted to its active form. The appearance of the active after a lag time of 2-4 hours is consistent with the unabsorbed material eventually reaching the colon and being converted by the colonic microflora.

TABLE 3

| | Parameter | | | |
|---|---|---|---|---|
| | Prodrug | | Amphetamine | |
| | AUC | | AUC | |
| Compound | (hr · ng/mL) | Tₘₐₓ (h) | (hr · ng/mL) | Tmax (h) |
| d-Amphetamine | — | — | 715 | 0.9 |
| 4 mg po | | | | |
| Compound 1, | 139 | 0.7 | 539 | 7.2 |
| 4 mg po | | | | |
| Compound 1, | 81.4 | 1.3 | 225 | 7.2 |
| 4 mg intranasal | | | | |

### Example 24

### In Vitro Conversion of Amphetamine Prodrugs to Amphetamine in Dog, Monkey and Human Faecal Homogenates and in Rat Caecal Contents

### Methodology

The amphetamine prodrugs were incubated at a concentration of 10 μM in an incubation mixture with dog, monkey and human faecal homogenates or rat caecal contents under anaerobic conditions at 37° C. for up to 24 hours. The incubates were sampled (0.5 ml) at 6 and 24 hours, inactivated using 1 ml acidified acetonitirile and snap frozen. Frozen samples were stored at −70° C. prior to analysis for the parent drug and pro-drug using a qualified LC-MS-MS assay.

### Results

The data are presented in Table 4. The data confirmed that the microflora are capable of efficient conversion of compounds 1, 5, 13, 16 and 17 to the active and are consistent with being the site of generation of the active drug in vivo. For some prodrugs e.g. compounds 5 and 17, conversion to the active was higher in human compared to dog faecal homogenates which was consistent with lower circulating levels of amphetamine following their administration to dogs.

TABLE 4

| | In Vitro Faecal/Caecal incubations | | | | | | | | | | | |
|---|---|---|---|---|---|---|---|---|---|---|---|---|
| | Dog | | | Monkey | | | Rat | | | Human | | |
| | Amphetamine (μM) | | | Amphetamine (μM) | | | Amphetamine (μM) | | | Amphetamine (μM) | | |
| Compound | C₂₄ | C₆ | C₀ | C₂₄ | C₆ | C₀ | C₂₄ | C₆ | C₀ | C₂₄ | C₆ | C₀ |
| 1 | 4.58 | 4.88 | 0.01 | 2.42 | 2.37 | BLQ | 2.27 | 2.38 | 0.01 | 2.32 | 2.50 | 0.04 |
| 2 | 3.24 | 1.35 | 0.55 | 3.09 | 1.44 | 0.29 | 1.29 | 0.62 | 0.32 | 1.77 | 0.78 | 0.23 |
| 4 | 0.37 | 0.09 | BLQ | NT | NT | NT | NT | NT | NT | 0.58 | 0.16 | BLQ |
| 5 | 0.55 | 0.46 | 0.43 | NT | NT | NT | NT | NT | NT | 4.84 | 1.76 | 0.53 |
| 6 | 0.07 | 0.008 | BLQ | NT | NT | NT | NT | NT | NT | 2.06 | 0.64 | BLQ |
| 7 | 0.12 | 0.09 | 0.07 | NT | NT | NT | NT | NT | NT | 0.11 | 0.08 | 0.06 |
| 8 | BLQ | BLQ | BLQ | NT | NT | NT | NT | NT | NT | BLQ | BLQ | BLQ |
| 9 | 0.36 | 0.16 | 0.10 | NT | NT | NT | NT | NT | NT | 1.21 | 0.49 | 0.10 |
| 10 | 0.278 | 0.173 | 0.099 | NT | NT | NT | NT | NT | NT | 0.66 | 0.35 | 0.132 |
| 11 | BLQ | BLQ | BLQ | NT | NT | NT | NT | NT | NT | BLQ | BLQ | BLQ |
| 12 | BLQ | BLQ | BLQ | NT | NT | NT | NT | NT | NT | BLQ | BLQ | BLQ |
| 13 | 0.64 | 0.61 | 0.05 | NT | NT | NT | NT | NT | NT | 0.33 | 0.30 | BLQ |
| 14 | 0.053 | 0.034 | BLQ | NT | NT | NT | NT | NT | NT | BLQ | BLQ | BLQ |
| 15 | 0.257 | 0.189 | BLQ | NT | NT | NT | NT | NT | NT | 0.069 | 0.028 | BLQ |
| 16 | 5.91 | 5.71 | BLQ | NT | NT | NT | NT | NT | NT | 1.67 | 0.84 | BLQ |
| 17 | 5.80 | 2.26 | BLQ | NT | NT | NT | NT | NT | NT | 8.00 | 3.68 | BLQ |
| 18 | 0.68 | 0.17 | BLQ | NT | NT | NT | NT | NT | NT | 0.32 | 0.10 | BLQ |
| 19 | 0.07 | 0.03 | BLQ | NT | NT | NT | NT | NT | NT | 0.07 | BLQ | BLQ |

BLQ = Below Limit of Quantification, NT = Not tested

### Example 25

### Interindividual Variability in Amphetamine Generation from Compound In Vitro in Human Fecal Homogenates

### Methodology

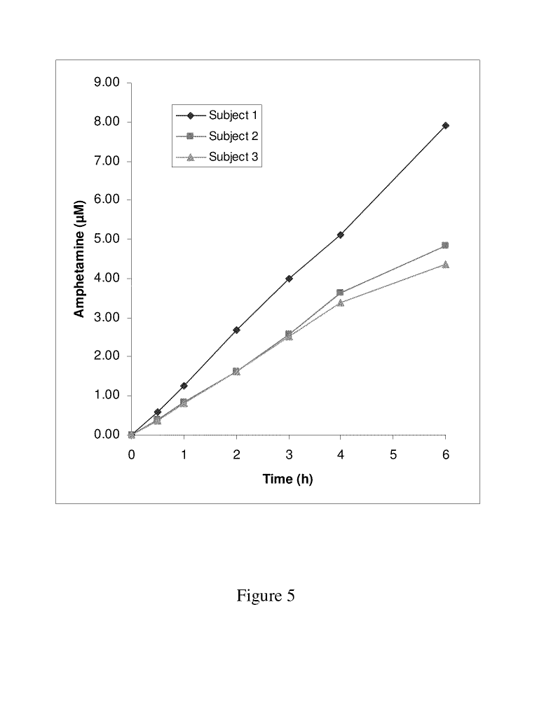

Compound 1 was incubated (100 μM) under anaerobic conditions at 37° C. at time points up to 6 hours at 37° C. with faecal homogenates prepared from three subjects. The generation of amphetamine from the prodrug in the three subjects showed a relatively modest variability as shown in FIG. 5.

All references, patents, and patent publications cited herein are hereby incorporated by reference.

1. An amphetamine prodrug comprising amphetamine conjugated at its amino terminus (i) to the acid group of a sugar acid to form an amide linkage, or (ii) to an amino group of an amino-saccharide to form a hydrazine linkage, wherein the sugar acid is not gulonic acid and the prodrug is not a metabolite of amphetamine, and optionally the sugar moiety (in open chain or closed ring form) in the sugar acid is separated from the amino terminus of the amphetamine by at least two atoms.

2. The amphetamine prodrug of claim 1, wherein the amphetamine is d-amphetamine.

3. (canceled)

4. The amphetamine prodrug of claim 1, wherein the amphetamine is conjugated to a sugar acid.

5. (canceled)

6. The amphetamine prodrug of claim 4, wherein the sugar acid is selected from muramic acid, lactobionic acid, glyceric acid, xylonic acid, gluconic acid, ketodeoxyoctulosonic acid (3-deoxy-d-manno-oct-2-ulosonic acid), galacturonic acid, tartaric acid, iduronic acid, galactonic acid (Mucic acid), glucaric acid, gluconic acid, and galonic acid.

7. The amphetamine prodrug of claim 4, wherein the sugar acid is N-acetyl D-muramic acid.

8. The amphetamine prodrug of claim 4, wherein the sugar acid is anhydro N-acetyl D-muramic acid.

9. The amphetamine prodrug of claim 4, where the sugar acid is lactobionic acid.

10. The amphetamine prodrug of claim 4, wherein the sugar acid has a substituent —NR³C(O)R⁴ at the alpha position adjacent to the aldehyde group, when the sugar group is in its open form, where R³ is hydrogen or C₁-C₄ alkyl, and R⁴ is C₁-C₄ alkyl.

11. The amphetamine prodrug of claim 10, wherein R⁴ is a straight chain C₁-C₄ alkyl.

12. The amphetamine prodrug of claim 10, wherein R³ is hydrogen and R⁴ is methyl.

13. The amphetamine prodrug of claim 10, wherein R³ is hydrogen and R⁴ is ethyl.

14. The amphetamine prodrug of claim 1, wherein the amphetamine is conjugated to an amino-saccharide.

15. The amphetamine prodrug of claim 14, wherein the amino-saccharide is an amino-monosaccharide.

16. The amphetamine prodrug of claim 14, wherein the amino-saccharide is selected from galactosamine, glucosamine, mannosamine, fucosamine, quinovosamine, lactosediamine, acosamine, bacillosamine, daunosamine, desosamine, forosamine, garosamine, kanosamine, kansosamine, mycaminose, mycosamine, perosamine, pneumosamine, purpurosamine, and rhodosamine.

17. (canceled)

18. The amphetamine prodrug of claim 17, wherein the sugar acid has an amino group, the amino moiety is protected, and the protecting group on the amino moiety is acetyl.

19. (canceled)

20. The amphetamine prodrug of claim 17, wherein one or more hydroxy moieties on the amino-saccharide are protected, and at least one hydroxy moiety is protected with a C₁-C₁₀ alkyl.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. A compound selected from

| | |
|---|---|
| 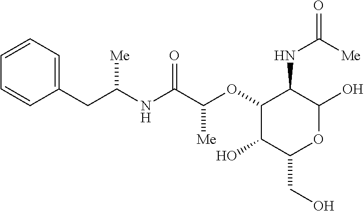 | Compound 1 |
| (2R)-2-(((3R,4R,5R,6R)-3-acetamido-2,5-dihydroxy-6-(hydroxymethyl)tetrahydro-2H-pyran-4- | |
| yl)oxy)-N-((S)-1-phenylpropan-2-yl)propanamide | |
| 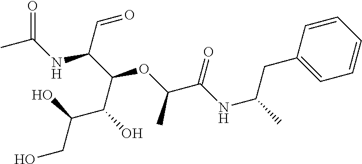 [SMILES: CC(=O)N[C@@H](C=O)[C@@H](O[C@H](C)C(=O)N[C@@H](C)Cc1ccccc1)[C@H](O)[C@H](O)CO] | Compound 1 |
| (R)-2-(((2R,3R,4R,5R)-2-acetamido-4,5,6-trihydroxy-1-oxohexan-3-yl)oxy)-N-((S)-1- | |
| phenylpropan-2-yl)propanamide | |
| 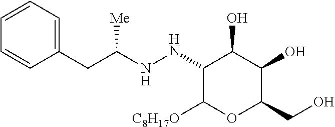 | Compound 2 |
| (2R,3R,4R,5R)-2-(hydroxymethyl)-6-(octyloxy)-5-(2-((S)-1-phenylpropan-2-yl) | |
| hydrazinyl)tetrahydro-2H-pyran-3,4-diol | |
| 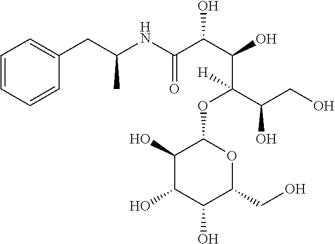 [SMILES: C[C@@H](Cc1ccccc1)NC(=O)[C@H](O)[C@@H](O)[C@H](O[C@@H]1O[C@H](CO)[C@H](O)[C@H](O)[C@H]1O)[C@H](O)CO] | Compound 5 |
| (2R,3R,4R,5R)-2,3,5,6-tetrahydroxy-N-((S)-1-phenylpropan-2-yl)-4-(((2S,3R,4S,5R,6R)-3,4,5- | |
| trihydroxy-6-(hydroxymethyl)tetrahydro-2H-pyran-2-yl)oxy)hexanamide | |
| 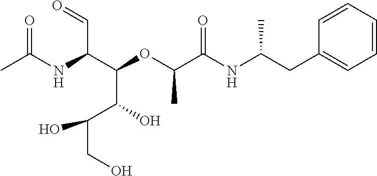 [SMILES: CC(=O)N[C@@H](C=O)[C@@H](O[C@H](C)C(=O)N[C@H](C)Cc1ccccc1)[C@H](O)[C@H](O)CO] | Compound 13 |
| (2R)-2-{[(2R,3R,4R,5R)-2-Acetamido-4,5,6-trihydroxy-1-oxohexan-3-yl]oxy}-N-[(2R)-1- | |
| phenylpropan-2-yl]propanamide | |
| 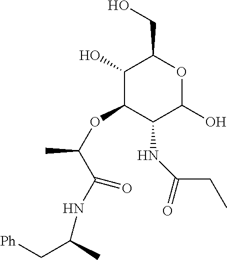 [SMILES: CCC(=O)N[C@H]1C(O)O[C@H](CO)[C@@H](O)[C@@H]1O[C@H](C)C(=O)N[C@@H](C)Cc1ccccc1] | Compound 16 |
| (2R)-2-[(3R,4R,5S)-2,5-dihydroxy-6-(hydroxymethyl)-3-(propanoylamino)tetrahydro pyran-4- | |
| yl]oxy-N-[(1S)-1-methyl-2-phenyl-ethyl]propanamide | |
| 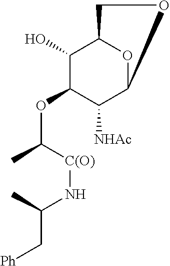 | Compound 17 |
| (2R)-2-[[(2S,3R,4R)-4-acetamido-2-hydroxy-6,8-dioxabicyclo[3.2.1]octan-3-yl]oxy]-N-[(1R)-1- | |
| methyl-2-phenyl-ethyl]propanamide | |

and tautomers thereof, and pharmaceutically acceptable salts thereof.

38. A compound of the formula: 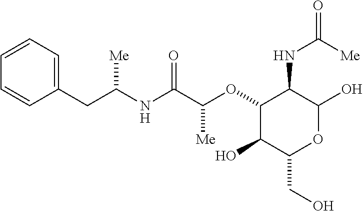 [SMILES: CC(=O)N[C@H]1C(O)O[C@H](CO)[C@@H](O)[C@@H]1O[C@H](C)C(=O)N[C@@H](C)Cc1ccccc1]
(S)-Amphetamine-(N-acetyl-D-muramic acid) amide
or a tautomer thereof, or a pharmaceutically acceptable thereof.

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. A method of treating a disorder treatable with an amphetamine in a subject in need thereof, comprising orally administering an amphetamine prodrug which releases the amphetamine upon cleavage by bacterial enzymes in the colon of the subject.

53. (canceled)

54. (canceled)

55. (canceled)

56. (canceled)

57. (canceled)

58. (canceled)

59. (canceled)

60. (canceled)

61. A method of treating a disorder treatable with an amphetamine in a subject in need thereof, comprising orally administering the amphetamine prodrug of claim 1.

62. The method of claim 61, wherein the disorder is attention deficit hyperactivity disorder.
